SEQUENCE LISTING (1224 sequences)

SEQ ID NO: 1 - RNA, 21 nt, synthetic construct
  source 1..21: mol_type = other RNA
tacagcttgagccgctccttg

SEQ ID NO: 2 - RNA, 21 nt, synthetic construct
  source 1..21: mol_type = other RNA
taacagcttgagccgctcctt

SEQ ID NO: 3 - RNA, 21 nt, synthetic construct
  source 1..21: mol_type = other RNA
tagaacagcttgagccgctcc

SEQ ID NO: 4 - RNA, 21 nt, synthetic construct
  source 1..21: mol_type = other RNA
taagaacagcttgagccgctc

SEQ ID NO: 5 - RNA, 21 nt, synthetic construct
  source 1..21: mol_type = other RNA
tgaagaacagcttgagccgct

SEQ ID NO: 6 - RNA, 21 nt, synthetic construct
  source 1..21: mol_type = other RNA
ttgaagaacagcttgagccgc

SEQ ID NO: 7 - RNA, 21 nt, synthetic construct
  source 1..21: mol_type = other RNA
tatgaagaacagcttgagccg

SEQ ID NO: 8 - RNA, 21 nt, synthetic construct
  source 1..21: mol_type = other RNA
tgatgaagaacagcttgagcc

SEQ ID NO: 9 - RNA, 21 nt, synthetic construct
  source 1..21: mol_type = other RNA
ttgatgaagaacagcttgagc

SEQ ID NO: 10 - RNA, 21 nt, synthetic construct
  source 1..21: mol_type = other RNA
tttgatgaagaacagcttgag

SEQ ID NO: 11 - RNA, 21 nt, synthetic construct
  source 1..21: mol_type = other RNA
ttttgatgaagaacagcttga

SEQ ID NO: 12 - RNA, 21 nt, synthetic construct
  source 1..21: mol_type = other RNA
ttgaacagccggatcctcagg

SEQ ID NO: 13 - RNA, 21 nt, synthetic construct
  source 1..21: mol_type = other RNA
tttgaacagccggatcctcag

SEQ ID NO: 14 - RNA, 21 nt, synthetic construct
  source 1..21: mol_type = other RNA
tgttgaacagccggatcctca

SEQ ID NO: 15 - RNA, 21 nt, synthetic construct
  source 1..21: mol_type = other RNA
tagttgaacagccggatcctc

SEQ ID NO: 16 - RNA, 21 nt, synthetic construct
  source 1..21: mol_type = other RNA
taagttgaacagccggatcct

SEQ ID NO: 17 - RNA, 21 nt, synthetic construct
  source 1..21: mol_type = other RNA
tgaagttgaacagccggatcc

SEQ ID NO: 18 - RNA, 21 nt, synthetic construct
  source 1..21: mol_type = other RNA
tagaagttgaacagccggatc

SEQ ID NO: 19 - RNA, 21 nt, synthetic construct
  source 1..21: mol_type = other RNA
tgagaagttgaacagccggat

SEQ ID NO: 20 - RNA, 21 nt, synthetic construct
  source 1..21: mol_type = other RNA
tggagaagttgaacagccgga

SEQ ID NO: 21 - RNA, 21 nt, synthetic construct
  source 1..21: mol_type = other RNA
tgggagaagttgaacagccgg

SEQ ID NO: 22 - RNA, 21 nt, synthetic construct
  source 1..21: mol_type = other RNA
tagggagaagttgaacagccg

SEQ ID NO: 23 - RNA, 21 nt, synthetic construct
  source 1..21: mol_type = other RNA
tccagcatccgatgcccaggg

SEQ ID NO: 24 - RNA, 21 nt, synthetic construct
  source 1..21: mol_type = other RNA
tcccagcatccgatgcccagg

SEQ ID NO: 25 - RNA, 21 nt, synthetic construct
  source 1..21: mol_type = other RNA
tctttgtagctgaggtagatg

SEQ ID NO: 26 - RNA, 21 nt, synthetic construct
  source 1..21: mol_type = other RNA
tcctttgtagctgaggtagat

SEQ ID NO: 27 - RNA, 21 nt, synthetic construct
  source 1..21: mol_type = other RNA
tgttgcctttgtagctgaggt

SEQ ID NO: 28 - RNA, 21 nt, synthetic construct
  source 1..21: mol_type = other RNA
ttgttgcctttgtagctgagg

SEQ ID NO: 29 - RNA, 21 nt, synthetic construct
  source 1..21: mol_type = other RNA
tatgttgcctttgtagctgag

SEQ ID NO: 30 - RNA, 21 nt, synthetic construct
  source 1..21: mol_type = other RNA
tgatgttgcctttgtagctga

SEQ ID NO: 31 - RNA, 21 nt, synthetic construct
  source 1..21: mol_type = other RNA
tagatgttgcctttgtagctg

SEQ ID NO: 32 - RNA, 21 nt, synthetic construct
  source 1..21: mol_type = other RNA
tcagatgttgcctttgtagct

SEQ ID NO: 33 - RNA, 21 nt, synthetic construct
  source 1..21: mol_type = other RNA
tccagatgttgcctttgtagc

SEQ ID NO: 34 - RNA, 21 nt, synthetic construct
  source 1..21: mol_type = other RNA
tcccagatgttgcctttgtag

SEQ ID NO: 35 - RNA, 21 nt, synthetic construct
  source 1..21: mol_type = other RNA
ttcccagatgttgcctttgta

SEQ ID NO: 36 - RNA, 21 nt, synthetic construct
  source 1..21: mol_type = other RNA
tctcccagatgttgcctttgt

SEQ ID NO: 37 - RNA, 21 nt, synthetic construct
  source 1..21: mol_type = other RNA
tgctcccagatgttgcctttg

SEQ ID NO: 38 - RNA, 21 nt, synthetic construct
  source 1..21: mol_type = other RNA
tgtgttgatcatctccaggac

SEQ ID NO: 39 - RNA, 21 nt, synthetic construct
  source 1..21: mol_type = other RNA
ttgatgatgaagggcagagtg

SEQ ID NO: 40 - RNA, 21 nt, synthetic construct
  source 1..21: mol_type = other RNA
tgtgatgatgaagggcagagt

SEQ ID NO: 41 - RNA, 21 nt, synthetic construct
  source 1..21: mol_type = other RNA
tcgtgatgatgaagggcagag

SEQ ID NO: 42 - RNA, 21 nt, synthetic construct
  source 1..21: mol_type = other RNA
ttgaacaggttccgcagcggc

SEQ ID NO: 43 - RNA, 21 nt, synthetic construct
  source 1..21: mol_type = other RNA
tatgaacaggttccgcagcgg

SEQ ID NO: 44 - RNA, 21 nt, synthetic construct
  source 1..21: mol_type = other RNA
tgatgaacaggttccgcagcg

SEQ ID NO: 45 - RNA, 21 nt, synthetic construct
  source 1..21: mol_type = other RNA
tggatgaacaggttccgcagc

SEQ ID NO: 46 - RNA, 21 nt, synthetic construct
  source 1..21: mol_type = other RNA
tgggatgaacaggttccgcag

SEQ ID NO: 47 - RNA, 21 nt, synthetic construct
  source 1..21: mol_type = other RNA
ttggcacggtggaagtcatta

SEQ ID NO: 48 - RNA, 21 nt, synthetic construct
  source 1..21: mol_type = other RNA
tatggcacggtggaagtcatt

SEQ ID NO: 49 - RNA, 21 nt, synthetic construct
  source 1..21: mol_type = other RNA
tgatggcacggtggaagtcat

SEQ ID NO: 50 - RNA, 21 nt, synthetic construct
  source 1..21: mol_type = other RNA
tggatggcacggtggaagtca

SEQ ID NO: 51 - RNA, 21 nt, synthetic construct
  source 1..21: mol_type = other RNA
taggatggcacggtggaagtc

SEQ ID NO: 52 - RNA, 21 nt, synthetic construct
  source 1..21: mol_type = other RNA
tcctggttgaacatggctgac

SEQ ID NO: 53 - RNA, 21 nt, synthetic construct
  source 1..21: mol_type = other RNA
tacagcagggtgcagaagagg

SEQ ID NO: 54 - RNA, 21 nt, synthetic construct
  source 1..21: mol_type = other RNA
tggcacagcagggtgcagaag

SEQ ID NO: 55 - RNA, 21 nt, synthetic construct
  source 1..21: mol_type = other RNA
tagtagaaggaggtcaggagg

SEQ ID NO: 56 - RNA, 21 nt, synthetic construct
  source 1..21: mol_type = other RNA
tgaagtagaaggaggtcagga

SEQ ID NO: 57 - RNA, 21 nt, synthetic construct
  source 1..21: mol_type = other RNA
tagaagtagaaggaggtcagg

SEQ ID NO: 58 - RNA, 21 nt, synthetic construct
  source 1..21: mol_type = other RNA
tgcagaagtagaaggaggtca

SEQ ID NO: 59 - RNA, 21 nt, synthetic construct
  source 1..21: mol_type = other RNA
ttgcagaagtagaaggaggtc

SEQ ID NO: 60 - RNA, 21 nt, synthetic construct
  source 1..21: mol_type = other RNA
tatgcagaagtagaaggaggt

SEQ ID NO: 61 - RNA, 21 nt, synthetic construct
  source 1..21: mol_type = other RNA
tgatgcagaagtagaaggagg

SEQ ID NO: 62 - RNA, 21 nt, synthetic construct
  source 1..21: mol_type = other RNA
tcgatgcagaagtagaaggag

SEQ ID NO: 63 - RNA, 21 nt, synthetic construct
  source 1..21: mol_type = other RNA
tacgatgcagaagtagaagga

SEQ ID NO: 64 - RNA, 21 nt, synthetic construct
  source 1..21: mol_type = other RNA
tgccagatcttgggcgtgacg

SEQ ID NO: 65 - RNA, 21 nt, synthetic construct
  source 1..21: mol_type = other RNA
tgcagtgggagcaccacgagg

SEQ ID NO: 66 - RNA, 21 nt, synthetic construct
  source 1..21: mol_type = other RNA
ttgcagtgggagcaccacgag

SEQ ID NO: 67 - RNA, 21 nt, synthetic construct
  source 1..21: mol_type = other RNA
tccatccagaggtagacgagc

SEQ ID NO: 68 - RNA, 21 nt, synthetic construct
  source 1..21: mol_type = other RNA
ttccatccagaggtagacgag

SEQ ID NO: 69 - RNA, 21 nt, synthetic construct
  source 1..21: mol_type = other RNA
tctccatccagaggtagacga

SEQ ID NO: 70 - RNA, 21 nt, synthetic construct
  source 1..21: mol_type = other RNA
tgctccatccagaggtagacg

SEQ ID NO: 71 - RNA, 21 nt, synthetic construct
  source 1..21: mol_type = other RNA
ttcaggaagtccatgagaagg

SEQ ID NO: 72 - RNA, 21 nt, synthetic construct
  source 1..21: mol_type = other RNA
tttcaggaagtccatgagaag

SEQ ID NO: 73 - RNA, 21 nt, synthetic construct
  source 1..21: mol_type = other RNA
tacaggatgaccacgtaatag

SEQ ID NO: 74 - RNA, 21 nt, synthetic construct
  source 1..21: mol_type = other RNA
tgacccgctgggaccacagag

SEQ ID NO: 75 - RNA, 21 nt, synthetic construct
  source 1..21: mol_type = other RNA
ttgacccgctgggaccacaga

SEQ ID NO: 76 - RNA, 21 nt, synthetic construct
  source 1..21: mol_type = other RNA
tatgacccgctgggaccacag

SEQ ID NO: 77 - RNA, 21 nt, synthetic construct
  source 1..21: mol_type = other RNA
tgatgacccgctgggaccaca

SEQ ID NO: 78 - RNA, 21 nt, synthetic construct
  source 1..21: mol_type = other RNA
tagatgacccgctgggaccac

SEQ ID NO: 79 - RNA, 21 nt, synthetic construct
  source 1..21: mol_type = other RNA
ttagatgacccgctgggacca

SEQ ID NO: 80 - RNA, 21 nt, synthetic construct
  source 1..21: mol_type = other RNA
tgtagatgacccgctgggacc

SEQ ID NO: 81 - RNA, 21 nt, synthetic construct
  source 1..21: mol_type = other RNA
tggtagatgacccgctgggac

SEQ ID NO: 82 - RNA, 21 nt, synthetic construct
  source 1..21: mol_type = other RNA
taggtagatgacccgctggga

SEQ ID NO: 83 - RNA, 21 nt, synthetic construct
  source 1..21: mol_type = other RNA
tgaggtagatgacccgctggg

SEQ ID NO: 84 - RNA, 21 nt, synthetic construct
  source 1..21: mol_type = other RNA
tggaggtagatgacccgctgg

SEQ ID NO: 85 - RNA, 21 nt, synthetic construct
  source 1..21: mol_type = other RNA
ttggaggtagatgacccgctg

SEQ ID NO: 86 - RNA, 21 nt, synthetic construct
  source 1..21: mol_type = other RNA
tctggaggtagatgacccgct

SEQ ID NO: 87 - RNA, 21 nt, synthetic construct
  source 1..21: mol_type = other RNA
tcctggaggtagatgacccgc

SEQ ID NO: 88 - RNA, 21 nt, synthetic construct
  source 1..21: mol_type = other RNA
tgccctggaggtagatgaccc

SEQ ID NO: 89 - RNA, 21 nt, synthetic construct
  source 1..21: mol_type = other RNA
tagccctggaggtagatgacc

SEQ ID NO: 90 - RNA, 21 nt, synthetic construct
  source 1..21: mol_type = other RNA
tgagccctggaggtagatgac

SEQ ID NO: 91 - RNA, 21 nt, synthetic construct
  source 1..21: mol_type = other RNA
tagagccctggaggtagatga

SEQ ID NO: 92 - RNA, 21 nt, synthetic construct
  source 1..21: mol_type = other RNA
tcagagccctggaggtagatg

SEQ ID NO: 93 - RNA, 21 nt, synthetic construct
  source 1..21: mol_type = other RNA
tgcagagccctggaggtagat

SEQ ID NO: 94 - RNA, 21 nt, synthetic construct
  source 1..21: mol_type = other RNA
ttcttggctcgcatgaggtcc

SEQ ID NO: 95 - RNA, 21 nt, synthetic construct
  source 1..21: mol_type = other RNA
tatcttggctcgcatgaggtc

SEQ ID NO: 96 - RNA, 21 nt, synthetic construct
  source 1..21: mol_type = other RNA
tcatcttggctcgcatgaggt

SEQ ID NO: 97 - RNA, 21 nt, synthetic construct
  source 1..21: mol_type = other RNA
tccatcttggctcgcatgagg

SEQ ID NO: 98 - RNA, 21 nt, synthetic construct
  source 1..21: mol_type = other RNA
ttccatcttggctcgcatgag

SEQ ID NO: 99 - RNA, 21 nt, synthetic construct
  source 1..21: mol_type = other RNA
tgtccatcttggctcgcatga

SEQ ID NO: 100 - RNA, 21 nt, synthetic construct
  source 1..21: mol_type = other RNA
ttgtccatcttggctcgcatg

SEQ ID NO: 101 - RNA, 21 nt, synthetic construct
  source 1..21: mol_type = other RNA
tttgtccatcttggctcgcat

SEQ ID NO: 102 - RNA, 21 nt, synthetic construct
  source 1..21: mol_type = other RNA
tccacgtggtcagcaaacttg

SEQ ID NO: 103 - RNA, 21 nt, synthetic construct
  source 1..21: mol_type = other RNA
tggcagatgcagttcagcgcc

SEQ ID NO: 104 - RNA, 21 nt, synthetic construct
  source 1..21: mol_type = other RNA
tgggcagatgcagttcagcgc

SEQ ID NO: 105 - RNA, 21 nt, synthetic construct
  source 1..21: mol_type = other RNA
tcgggcagatgcagttcagcg

SEQ ID NO: 106 - RNA, 21 nt, synthetic construct
  source 1..21: mol_type = other RNA
tccgggcagatgcagttcagc

SEQ ID NO: 107 - RNA, 21 nt, synthetic construct
  source 1..21: mol_type = other RNA
tgccgggcagatgcagttcag

SEQ ID NO: 108 - RNA, 21 nt, synthetic construct
  source 1..21: mol_type = other RNA
tagggtgatgagggtggaggt

SEQ ID NO: 109 - RNA, 21 nt, synthetic construct
  source 1..21: mol_type = other RNA
tcagggtgatgagggtggagg

SEQ ID NO: 110 - RNA, 21 nt, synthetic construct
  source 1..21: mol_type = other RNA
tgcagggtgatgagggtggag

SEQ ID NO: 111 - RNA, 21 nt, synthetic construct
  source 1..21: mol_type = other RNA
tgcaccagcagggtgatgagg

SEQ ID NO: 112 - RNA, 21 nt, synthetic construct
  source 1..21: mol_type = other RNA
tgacgtgtgcaccagcagggt

SEQ ID NO: 113 - RNA, 21 nt, synthetic construct
  source 1..21: mol_type = other RNA
ttgcgctgccactgctccgga

SEQ ID NO: 114 - RNA, 21 nt, synthetic construct
  source 1..21: mol_type = other RNA
ttgaagctcttgccctcgtac

SEQ ID NO: 115 - RNA, 21 nt, synthetic construct
  source 1..21: mol_type = other RNA
ttaggtgaagctcttgccctc

SEQ ID NO: 116 - RNA, 21 nt, synthetic construct
  source 1..21: mol_type = other RNA
tgtaggtgaagctcttgccct

SEQ ID NO: 117 - RNA, 21 nt, synthetic construct
  source 1..21: mol_type = other RNA
tcgtaggtgaagctcttgccc

SEQ ID NO: 118 - RNA, 21 nt, synthetic construct
  source 1..21: mol_type = other RNA
tgcgtaggtgaagctcttgcc

SEQ ID NO: 119 - RNA, 21 nt, synthetic construct
  source 1..21: mol_type = other RNA
tgcccgatgaggcacacgcca

SEQ ID NO: 120 - RNA, 21 nt, synthetic construct
  source 1..21: mol_type = other RNA
tcagcccgatgaggcacacgc

SEQ ID NO: 121 - RNA, 21 nt, synthetic construct
  source 1..21: mol_type = other RNA
ttcagcccgatgaggcacacg

SEQ ID NO: 122 - RNA, 21 nt, synthetic construct
  source 1..21: mol_type = other RNA
tttcagcccgatgaggcacac

SEQ ID NO: 123 - RNA, 21 nt, synthetic construct
  source 1..21: mol_type = other RNA
tgcttcagcccgatgaggcac

SEQ ID NO: 124 - RNA, 21 nt, synthetic construct
  source 1..21: mol_type = other RNA
tcgcttcagcccgatgaggca

SEQ ID NO: 125 - RNA, 21 nt, synthetic construct
  source 1..21: mol_type = other RNA
tagttctcctccttggtgatg

SEQ ID NO: 126 - RNA, 21 nt, synthetic construct
  source 1..21: mol_type = other RNA
tgagttctcctccttggtgat

SEQ ID NO: 127 - RNA, 21 nt, synthetic construct
  source 1..21: mol_type = other RNA
tacaggcaggaggtggcacag

SEQ ID NO: 128 - RNA, 21 nt, synthetic construct
  source 1..21: mol_type = other RNA
tcacaggcaggaggtggcaca

SEQ ID NO: 129 - RNA, 21 nt, synthetic construct
  source 1..21: mol_type = other RNA
ttcacaggcaggaggtggcac

SEQ ID NO: 130 - RNA, 21 nt, synthetic construct
  source 1..21: mol_type = other RNA
tttcacaggcaggaggtggca

SEQ ID NO: 131 - RNA, 21 nt, synthetic construct
  source 1..21: mol_type = other RNA
tttgcagcccttgtccagccg

SEQ ID NO: 132 - RNA, 21 nt, synthetic construct
  source 1..21: mol_type = other RNA
tgcttgttcttgaacccgtag

SEQ ID NO: 133 - RNA, 21 nt, synthetic construct
  source 1..21: mol_type = other RNA
tagcttgttcttgaacccgta

SEQ ID NO: 134 - RNA, 21 nt, synthetic construct
  source 1..21: mol_type = other RNA
tcagcttgttcttgaacccgt

SEQ ID NO: 135 - RNA, 21 nt, synthetic construct
  source 1..21: mol_type = other RNA
ttcagcttgttcttgaacccg

SEQ ID NO: 136 - RNA, 21 nt, synthetic construct
  source 1..21: mol_type = other RNA
tatcagcttgttcttgaaccc

SEQ ID NO: 137 - RNA, 21 nt, synthetic construct
  source 1..21: mol_type = other RNA
tgcacgatgaagttgtacagc

SEQ ID NO: 138 - RNA, 21 nt, synthetic construct
  source 1..21: mol_type = other RNA
tccaggaagtggtggtcgggc

SEQ ID NO: 139 - RNA, 21 nt, synthetic construct
  source 1..21: mol_type = other RNA
ttccaggaagtggtggtcggg

SEQ ID NO: 140 - RNA, 21 nt, synthetic construct
  source 1..21: mol_type = other RNA
ttccatgtagtagaccatggg

SEQ ID NO: 141 - RNA, 21 nt, synthetic construct
  source 1..21: mol_type = other RNA
tctccatgtagtagaccatgg

SEQ ID NO: 142 - RNA, 21 nt, synthetic construct
  source 1..21: mol_type = other RNA
tcctccatgtagtagaccatg

SEQ ID NO: 143 - RNA, 21 nt, synthetic construct
  source 1..21: mol_type = other RNA
ttgtccaggttgtccacagag

SEQ ID NO: 144 - RNA, 21 nt, synthetic construct
  source 1..21: mol_type = other RNA
tgctgtccaggttgtccacag

SEQ ID NO: 145 - RNA, 21 nt, synthetic construct
  source 1..21: mol_type = other RNA
tccttgtccaccaccaccagg

SEQ ID NO: 146 - RNA, 21 nt, synthetic construct
  source 1..21: mol_type = other RNA
ttccttgtccaccaccaccag

SEQ ID NO: 147 - RNA, 21 nt, synthetic construct
  source 1..21: mol_type = other RNA
tctccttgtccaccaccacca

SEQ ID NO: 148 - RNA, 21 nt, synthetic construct
  source 1..21: mol_type = other RNA
tgctctccttgtccaccacca

SEQ ID NO: 149 - RNA, 21 nt, synthetic construct
  source 1..21: mol_type = other RNA
ttgctctccttgtccaccacc

SEQ ID NO: 150 - RNA, 21 nt, synthetic construct
  source 1..21: mol_type = other RNA
tgtgctctccttgtccaccac

SEQ ID NO: 151 - RNA, 21 nt, synthetic construct
  source 1..21: mol_type = other RNA
ttgacgatggtcttggcgtcc

SEQ ID NO: 152 - RNA, 21 nt, synthetic construct
  source 1..21: mol_type = other RNA
tttgacgatggtcttggcgtc

SEQ ID NO: 153 - RNA, 21 nt, synthetic construct
  source 1..21: mol_type = other RNA
tccgtggtgatgctgaggctg

SEQ ID NO: 154 - RNA, 21 nt, synthetic construct
  source 1..21: mol_type = other RNA
tctccgtggtgatgctgaggc

SEQ ID NO: 155 - RNA, 21 nt, synthetic construct
  source 1..21: mol_type = other RNA
tgctccgtggtgatgctgagg

SEQ ID NO: 156 - RNA, 21 nt, synthetic construct
  source 1..21: mol_type = other RNA
tgagctccgtggtgatgctga

SEQ ID NO: 157 - RNA, 21 nt, synthetic construct
  source 1..21: mol_type = other RNA
ttgagctccgtggtgatgctg

SEQ ID NO: 158 - RNA, 21 nt, synthetic construct
  source 1..21: mol_type = other RNA
tgtgagctccgtggtgatgct

SEQ ID NO: 159 - RNA, 21 nt, synthetic construct
  source 1..21: mol_type = other RNA
tgcatgttggaagggtgggtg

SEQ ID NO: 160 - RNA, 21 nt, synthetic construct
  source 1..21: mol_type = other RNA
tcgcatgttggaagggtgggt

SEQ ID NO: 161 - RNA, 21 nt, synthetic construct
  source 1..21: mol_type = other RNA
tagtagctgtccttggcgcgg

SEQ ID NO: 162 - RNA, 21 nt, synthetic construct
  source 1..21: mol_type = other RNA
tgagtagctgtccttggcgcg

SEQ ID NO: 163 - RNA, 21 nt, synthetic construct
  source 1..21: mol_type = other RNA
tagagtagctgtccttggcgc

SEQ ID NO: 164 - RNA, 21 nt, synthetic construct
  source 1..21: mol_type = other RNA
tgagagtagctgtccttggcg

SEQ ID NO: 165 - RNA, 21 nt, synthetic construct
  source 1..21: mol_type = other RNA
tagagagtagctgtccttggc

SEQ ID NO: 166 - RNA, 21 nt, synthetic construct
  source 1..21: mol_type = other RNA
tcagagagtagctgtccttgg

SEQ ID NO: 167 - RNA, 21 nt, synthetic construct
  source 1..21: mol_type = other RNA
tccagagagtagctgtccttg

SEQ ID NO: 168 - RNA, 21 nt, synthetic construct
  source 1..21: mol_type = other RNA
tgccagagagtagctgtcctt

SEQ ID NO: 169 - RNA, 21 nt, synthetic construct
  source 1..21: mol_type = other RNA
tagccagagagtagctgtcct

SEQ ID NO: 170 - RNA, 21 nt, synthetic construct
  source 1..21: mol_type = other RNA
tgaaagagccagagagtagct

SEQ ID NO: 171 - RNA, 21 nt, synthetic construct
  source 1..21: mol_type = other RNA
tggaaagagccagagagtagc

SEQ ID NO: 172 - RNA, 21 nt, synthetic construct
  source 1..21: mol_type = other RNA
ttggaaagagccagagagtag

SEQ ID NO: 173 - RNA, 21 nt, synthetic construct
  source 1..21: mol_type = other RNA
tttggaaagagccagagagta

SEQ ID NO: 174 - RNA, 21 nt, synthetic construct
  source 1..21: mol_type = other RNA
ttttggaaagagccagagagt

SEQ ID NO: 175 - RNA, 21 nt, synthetic construct
  source 1..21: mol_type = other RNA
tgtttggaaagagccagagag

SEQ ID NO: 176 - RNA, 21 nt, synthetic construct
  source 1..21: mol_type = other RNA
tagtttggaaagagccagaga

SEQ ID NO: 177 - RNA, 21 nt, synthetic construct
  source 1..21: mol_type = other RNA
taggccaggttggagccattc

SEQ ID NO: 178 - RNA, 21 nt, synthetic construct
  source 1..21: mol_type = other RNA
tgaaggccaggttggagccat

SEQ ID NO: 179 - RNA, 21 nt, synthetic construct
  source 1..21: mol_type = other RNA
tacatgaaggccaggttggag

SEQ ID NO: 180 - RNA, 21 nt, synthetic construct
  source 1..21: mol_type = other RNA
taacatgaaggccaggttgga

SEQ ID NO: 181 - RNA, 21 nt, synthetic construct
  source 1..21: mol_type = other RNA
tgaacatgaaggccaggttgg

SEQ ID NO: 182 - RNA, 21 nt, synthetic construct
  source 1..21: mol_type = other RNA
tggaacatgaaggccaggttg

SEQ ID NO: 183 - RNA, 21 nt, synthetic construct
  source 1..21: mol_type = other RNA
tgcggaacatgaaggccaggt

SEQ ID NO: 184 - RNA, 21 nt, synthetic construct
  source 1..21: mol_type = other RNA
tgcaggcggaacatgaaggcc

SEQ ID NO: 185 - RNA, 21 nt, synthetic construct
  source 1..21: mol_type = other RNA
tggcaggcggaacatgaaggc

SEQ ID NO: 186 - RNA, 21 nt, synthetic construct
  source 1..21: mol_type = other RNA
tgaaggactggtagagcagtg

SEQ ID NO: 187 - RNA, 21 nt, synthetic construct
  source 1..21: mol_type = other RNA
tcgaaggactggtagagcagt

SEQ ID NO: 188 - RNA, 21 nt, synthetic construct
  source 1..21: mol_type = other RNA
tacgaaggactggtagagcag

SEQ ID NO: 189 - RNA, 21 nt, synthetic construct
  source 1..21: mol_type = other RNA
tcacgaaggactggtagagca

SEQ ID NO: 190 - RNA, 21 nt, synthetic construct
  source 1..21: mol_type = other RNA
ttcacgaaggactggtagagc

SEQ ID NO: 191 - RNA, 21 nt, synthetic construct
  source 1..21: mol_type = other RNA
tttcacgaaggactggtagag

SEQ ID NO: 192 - RNA, 21 nt, synthetic construct
  source 1..21: mol_type = other RNA
tcttcacgaaggactggtaga

SEQ ID NO: 193 - RNA, 21 nt, synthetic construct
  source 1..21: mol_type = other RNA
tccttcacgaaggactggtag

SEQ ID NO: 194 - RNA, 21 nt, synthetic construct
  source 1..21: mol_type = other RNA
tatgtagtccttcacgaagga

SEQ ID NO: 195 - RNA, 21 nt, synthetic construct
  source 1..21: mol_type = other RNA
tcatgtagtccttcacgaagg

SEQ ID NO: 196 - RNA, 21 nt, synthetic construct
  source 1..21: mol_type = other RNA
ttcatgtagtccttcacgaag

SEQ ID NO: 197 - RNA, 21 nt, synthetic construct
  source 1..21: mol_type = other RNA
tatcatgtagtccttcacgaa

SEQ ID NO: 198 - RNA, 21 nt, synthetic construct
  source 1..21: mol_type = other RNA
tgatcatgtagtccttcacga

SEQ ID NO: 199 - RNA, 21 nt, synthetic construct
  source 1..21: mol_type = other RNA
ttgatcatgtagtccttcacg

SEQ ID NO: 200 - RNA, 21 nt, synthetic construct
  source 1..21: mol_type = other RNA
tgtgatcatgtagtccttcac

SEQ ID NO: 201 - RNA, 21 nt, synthetic construct
  source 1..21: mol_type = other RNA
tggtgatcatgtagtccttca

SEQ ID NO: 202 - RNA, 21 nt, synthetic construct
  source 1..21: mol_type = other RNA
ttggtgatcatgtagtccttc

SEQ ID NO: 203 - RNA, 21 nt, synthetic construct
  source 1..21: mol_type = other RNA
tgatggtgatcatgtagtcct

SEQ ID NO: 204 - RNA, 21 nt, synthetic construct
  source 1..21: mol_type = other RNA
ttgatggtgatcatgtagtcc

SEQ ID NO: 205 - RNA, 21 nt, synthetic construct
  source 1..21: mol_type = other RNA
tgtgatggtgatcatgtagtc

SEQ ID NO: 206 - RNA, 21 nt, synthetic construct
  source 1..21: mol_type = other RNA
tggtgatggtgatcatgtagt

SEQ ID NO: 207 - RNA, 21 nt, synthetic construct
  source 1..21: mol_type = other RNA
tgcggatccacaggtcgccct

SEQ ID NO: 208 - RNA, 21 nt, synthetic construct
  source 1..21: mol_type = other RNA
tggaagaggcggccgtacgtg

SEQ ID NO: 209 - RNA, 21 nt, synthetic construct
  source 1..21: mol_type = other RNA
tctggaagaggcggccgtacg

SEQ ID NO: 210 - RNA, 21 nt, synthetic construct
  source 1..21: mol_type = other RNA
ttctggaagaggcggccgtac

SEQ ID NO: 211 - RNA, 21 nt, synthetic construct
  source 1..21: mol_type = other RNA
tttctggaagaggcggccgta

SEQ ID NO: 212 - RNA, 21 nt, synthetic construct
  source 1..21: mol_type = other RNA
tggcgctggaggagcagagct

SEQ ID NO: 213 - RNA, 21 nt, synthetic construct
  source 1..21: mol_type = other RNA
tctcggcgctggaggagcaga

SEQ ID NO: 214 - RNA, 21 nt, synthetic construct
  source 1..21: mol_type = other RNA
tatctcggcgctggaggagca

SEQ ID NO: 215 - RNA, 21 nt, synthetic construct
  source 1..21: mol_type = other RNA
tgatctcggcgctggaggagc

SEQ ID NO: 216 - RNA, 21 nt, synthetic construct
  source 1..21: mol_type = other RNA
tggatctcggcgctggaggag

SEQ ID NO: 217 - RNA, 21 nt, synthetic construct
  source 1..21: mol_type = other RNA
tgggatctcggcgctggagga

SEQ ID NO: 218 - RNA, 21 nt, synthetic construct
  source 1..21: mol_type = other RNA
ttgggagctgctggccacgtg

SEQ ID NO: 219 - RNA, 16 nt, synthetic construct
  source 1..16: mol_type = other RNA
agcggctcaagctgta

SEQ ID NO: 220 - RNA, 16 nt, synthetic construct
  source 1..16: mol_type = other RNA
gcggctcaagctgtta

SEQ ID NO: 221 - RNA, 16 nt, synthetic construct
  source 1..16: mol_type = other RNA
ggctcaagctgttcta

SEQ ID NO: 222 - RNA, 16 nt, synthetic construct
  source 1..16: mol_type = other RNA
gctcaagctgttctta

SEQ ID NO: 223 - RNA, 16 nt, synthetic construct
  source 1..16: mol_type = other RNA
ctcaagctgttcttca

SEQ ID NO: 224 - RNA, 16 nt, synthetic construct
  source 1..16: mol_type = other RNA
tcaagctgttcttcaa

SEQ ID NO: 225 - RNA, 16 nt, synthetic construct
  source 1..16: mol_type = other RNA
caagctgttcttcata

SEQ ID NO: 226 - RNA, 16 nt, synthetic construct
  source 1..16: mol_type = other RNA
aagctgttcttcatca

SEQ ID NO: 227 - RNA, 16 nt, synthetic construct
  source 1..16: mol_type = other RNA
agctgttcttcatcaa

SEQ ID NO: 228 - RNA, 16 nt, synthetic construct
  source 1..16: mol_type = other RNA
gctgttcttcatcaaa

SEQ ID NO: 229 - RNA, 16 nt, synthetic construct
  source 1..16: mol_type = other RNA
ctgttcttcatcaaaa

SEQ ID NO: 230 - RNA, 16 nt, synthetic construct
  source 1..16: mol_type = other RNA
ggatccggctgttcaa

SEQ ID NO: 231 - RNA, 16 nt, synthetic construct
  source 1..16: mol_type = other RNA
gatccggctgttcaaa

SEQ ID NO: 232 - RNA, 16 nt, synthetic construct
  source 1..16: mol_type = other RNA
atccggctgttcaaca

SEQ ID NO: 233 - RNA, 16 nt, synthetic construct
  source 1..16: mol_type = other RNA
tccggctgttcaacta

SEQ ID NO: 234 - RNA, 16 nt, synthetic construct
  source 1..16: mol_type = other RNA
ccggctgttcaactta

SEQ ID NO: 235 - RNA, 16 nt, synthetic construct
  source 1..16: mol_type = other RNA
cggctgttcaacttca

SEQ ID NO: 236 - RNA, 16 nt, synthetic construct
  source 1..16: mol_type = other RNA
ggctgttcaacttcta

SEQ ID NO: 237 - RNA, 16 nt, synthetic construct
  source 1..16: mol_type = other RNA
gctgttcaacttctca

SEQ ID NO: 238 - RNA, 16 nt, synthetic construct
  source 1..16: mol_type = other RNA
ctgttcaacttctcca

SEQ ID NO: 239 - RNA, 16 nt, synthetic construct
  source 1..16: mol_type = other RNA
tgttcaacttctccca

SEQ ID NO: 240 - RNA, 16 nt, synthetic construct
  source 1..16: mol_type = other RNA
gttcaacttctcccta

SEQ ID NO: 241 - RNA, 16 nt, synthetic construct
  source 1..16: mol_type = other RNA
ggcatcggatgctgga

SEQ ID NO: 242 - RNA, 16 nt, synthetic construct
  source 1..16: mol_type = other RNA
gcatcggatgctggga

SEQ ID NO: 243 - RNA, 16 nt, synthetic construct
  source 1..16: mol_type = other RNA
acctcagctacaaaga

SEQ ID NO: 244 - RNA, 16 nt, synthetic construct
  source 1..16: mol_type = other RNA
cctcagctacaaagga

SEQ ID NO: 245 - RNA, 16 nt, synthetic construct
  source 1..16: mol_type = other RNA
agctacaaaggcaaca

SEQ ID NO: 246 - RNA, 16 nt, synthetic construct
  source 1..16: mol_type = other RNA
gctacaaaggcaacaa

SEQ ID NO: 247 - RNA, 16 nt, synthetic construct
  source 1..16: mol_type = other RNA
ctacaaaggcaacata

SEQ ID NO: 248 - RNA, 16 nt, synthetic construct
  source 1..16: mol_type = other RNA
tacaaaggcaacatca

SEQ ID NO: 249 - RNA, 16 nt, synthetic construct
  source 1..16: mol_type = other RNA
acaaaggcaacatcta

SEQ ID NO: 250 - RNA, 16 nt, synthetic construct
  source 1..16: mol_type = other RNA
caaaggcaacatctga

SEQ ID NO: 251 - RNA, 16 nt, synthetic construct
  source 1..16: mol_type = other RNA
aaaggcaacatctgga

SEQ ID NO: 252 - RNA, 16 nt, synthetic construct
  source 1..16: mol_type = other RNA
aaggcaacatctggga

SEQ ID NO: 253 - RNA, 16 nt, synthetic construct
  source 1..16: mol_type = other RNA
aggcaacatctgggaa

SEQ ID NO: 254 - RNA, 16 nt, synthetic construct
  source 1..16: mol_type = other RNA
ggcaacatctgggaga

SEQ ID NO: 255 - RNA, 16 nt, synthetic construct
  source 1..16: mol_type = other RNA
gcaacatctgggagca

SEQ ID NO: 256 - RNA, 16 nt, synthetic construct
  source 1..16: mol_type = other RNA
ggagatgatcaacaca

SEQ ID NO: 257 - RNA, 16 nt, synthetic construct
  source 1..16: mol_type = other RNA
tgcccttcatcatcaa

SEQ ID NO: 258 - RNA, 16 nt, synthetic construct
  source 1..16: mol_type = other RNA
gcccttcatcatcaca

SEQ ID NO: 259 - RNA, 16 nt, synthetic construct
  source 1..16: mol_type = other RNA
cccttcatcatcacga

SEQ ID NO: 260 - RNA, 16 nt, synthetic construct
  source 1..16: mol_type = other RNA
tgcggaacctgttcaa

SEQ ID NO: 261 - RNA, 16 nt, synthetic construct
  source 1..16: mol_type = other RNA
gcggaacctgttcata

SEQ ID NO: 262 - RNA, 16 nt, synthetic construct
  source 1..16: mol_type = other RNA
cggaacctgttcatca

SEQ ID NO: 263 - RNA, 16 nt, synthetic construct
  source 1..16: mol_type = other RNA
ggaacctgttcatcca

SEQ ID NO: 264 - RNA, 16 nt, synthetic construct
  source 1..16: mol_type = other RNA
gaacctgttcatccca

SEQ ID NO: 265 - RNA, 16 nt, synthetic construct
  source 1..16: mol_type = other RNA
acttccaccgtgccaa

SEQ ID NO: 266 - RNA, 16 nt, synthetic construct
  source 1..16: mol_type = other RNA
cttccaccgtgccata

SEQ ID NO: 267 - RNA, 16 nt, synthetic construct
  source 1..16: mol_type = other RNA
ttccaccgtgccatca

SEQ ID NO: 268 - RNA, 16 nt, synthetic construct
  source 1..16: mol_type = other RNA
tccaccgtgccatcca

SEQ ID NO: 269 - RNA, 16 nt, synthetic construct
  source 1..16: mol_type = other RNA
ccaccgtgccatccta

SEQ ID NO: 270 - RNA, 16 nt, synthetic construct
  source 1..16: mol_type = other RNA
ccatgttcaaccagga

SEQ ID NO: 271 - RNA, 16 nt, synthetic construct
  source 1..16: mol_type = other RNA
tctgcaccctgctgta

SEQ ID NO: 272 - RNA, 16 nt, synthetic construct
  source 1..16: mol_type = other RNA
gcaccctgctgtgcca

SEQ ID NO: 273 - RNA, 16 nt, synthetic construct
  source 1..16: mol_type = other RNA
tgacctccttctacta

SEQ ID NO: 274 - RNA, 16 nt, synthetic construct
  source 1..16: mol_type = other RNA
acctccttctacttca

SEQ ID NO: 275 - RNA, 16 nt, synthetic construct
  source 1..16: mol_type = other RNA
cctccttctacttcta

SEQ ID NO: 276 - RNA, 16 nt, synthetic construct
  source 1..16: mol_type = other RNA
tccttctacttctgca

SEQ ID NO: 277 - RNA, 16 nt, synthetic construct
  source 1..16: mol_type = other RNA
ccttctacttctgcaa

SEQ ID NO: 278 - RNA, 16 nt, synthetic construct
  source 1..16: mol_type = other RNA
cttctacttctgcata

SEQ ID NO: 279 - RNA, 16 nt, synthetic construct
  source 1..16: mol_type = other RNA
ttctacttctgcatca

SEQ ID NO: 280 - RNA, 16 nt, synthetic construct
  source 1..16: mol_type = other RNA
tctacttctgcatcga

SEQ ID NO: 281 - RNA, 16 nt, synthetic construct
  source 1..16: mol_type = other RNA
ctacttctgcatcgta

SEQ ID NO: 282 - RNA, 16 nt, synthetic construct
  source 1..16: mol_type = other RNA
cgcccaagatctggca

SEQ ID NO: 283 - RNA, 16 nt, synthetic construct
  source 1..16: mol_type = other RNA
tggtgctcccactgca

SEQ ID NO: 284 - RNA, 16 nt, synthetic construct
  source 1..16: mol_type = other RNA
ggtgctcccactgcaa

SEQ ID NO: 285 - RNA, 16 nt, synthetic construct
  source 1..16: mol_type = other RNA
tctacctctggatgga

SEQ ID NO: 286 - RNA, 16 nt, synthetic construct
  source 1..16: mol_type = other RNA
ctacctctggatggaa

SEQ ID NO: 287 - RNA, 16 nt, synthetic construct
  source 1..16: mol_type = other RNA
tacctctggatggaga

SEQ ID NO: 288 - RNA, 16 nt, synthetic construct
  source 1..16: mol_type = other RNA
acctctggatggagca

SEQ ID NO: 289 - RNA, 16 nt, synthetic construct
  source 1..16: mol_type = other RNA
tcatggacttcctgaa

SEQ ID NO: 290 - RNA, 16 nt, synthetic construct
  source 1..16: mol_type = other RNA
catggacttcctgaaa

SEQ ID NO: 291 - RNA, 16 nt, synthetic construct
  source 1..16: mol_type = other RNA
acgtggtcatcctgta

SEQ ID NO: 292 - RNA, 16 nt, synthetic construct
  source 1..16: mol_type = other RNA
tggtcccagcgggtca

SEQ ID NO: 293 - RNA, 16 nt, synthetic construct
  source 1..16: mol_type = other RNA
ggtcccagcgggtcaa

SEQ ID NO: 294 - RNA, 16 nt, synthetic construct
  source 1..16: mol_type = other RNA
gtcccagcgggtcata

SEQ ID NO: 295 - RNA, 16 nt, synthetic construct
  source 1..16: mol_type = other RNA
tcccagcgggtcatca

SEQ ID NO: 296 - RNA, 16 nt, synthetic construct
  source 1..16: mol_type = other RNA
cccagcgggtcatcta

SEQ ID NO: 297 - RNA, 16 nt, synthetic construct
  source 1..16: mol_type = other RNA
ccagcgggtcatctaa

SEQ ID NO: 298 - RNA, 16 nt, synthetic construct
  source 1..16: mol_type = other RNA
cagcgggtcatctaca

SEQ ID NO: 299 - RNA, 16 nt, synthetic construct
  source 1..16: mol_type = other RNA
agcgggtcatctacca

SEQ ID NO: 300 - RNA, 16 nt, synthetic construct
  source 1..16: mol_type = other RNA
gcgggtcatctaccta

SEQ ID NO: 301 - RNA, 16 nt, synthetic construct
  source 1..16: mol_type = other RNA
cgggtcatctacctca

SEQ ID NO: 302 - RNA, 16 nt, synthetic construct
  source 1..16: mol_type = other RNA
gggtcatctacctcca

SEQ ID NO: 303 - RNA, 16 nt, synthetic construct
  source 1..16: mol_type = other RNA
ggtcatctacctccaa

SEQ ID NO: 304 - RNA, 16 nt, synthetic construct
  source 1..16: mol_type = other RNA
gtcatctacctccaga

SEQ ID NO: 305 - RNA, 16 nt, synthetic construct
  source 1..16: mol_type = other RNA
tcatctacctccagga

SEQ ID NO: 306 - RNA, 16 nt, synthetic construct
  source 1..16: mol_type = other RNA
atctacctccagggca

SEQ ID NO: 307 - RNA, 16 nt, synthetic construct
  source 1..16: mol_type = other RNA
tctacctccagggcta

SEQ ID NO: 308 - RNA, 16 nt, synthetic construct
  source 1..16: mol_type = other RNA
ctacctccagggctca

SEQ ID NO: 309 - RNA, 16 nt, synthetic construct
  source 1..16: mol_type = other RNA
tacctccagggctcta

SEQ ID NO: 310 - RNA, 16 nt, synthetic construct
  source 1..16: mol_type = other RNA
acctccagggctctga

SEQ ID NO: 311 - RNA, 16 nt, synthetic construct
  source 1..16: mol_type = other RNA
cctccagggctctgca

SEQ ID NO: 312 - RNA, 16 nt, synthetic construct
  source 1..16: mol_type = other RNA
tcatgcgagccaagaa

SEQ ID NO: 313 - RNA, 16 nt, synthetic construct
  source 1..16: mol_type = other RNA
catgcgagccaagata

SEQ ID NO: 314 - RNA, 16 nt, synthetic construct
  source 1..16: mol_type = other RNA
atgcgagccaagatga

SEQ ID NO: 315 - RNA, 16 nt, synthetic construct
  source 1..16: mol_type = other RNA
tgcgagccaagatgga

SEQ ID NO: 316 - RNA, 16 nt, synthetic construct
  source 1..16: mol_type = other RNA
gcgagccaagatggaa

SEQ ID NO: 317 - RNA, 16 nt, synthetic construct
  source 1..16: mol_type = other RNA
cgagccaagatggaca

SEQ ID NO: 318 - RNA, 16 nt, synthetic construct
  source 1..16: mol_type = other RNA
gagccaagatggacaa

SEQ ID NO: 319 - RNA, 16 nt, synthetic construct
  source 1..16: mol_type = other RNA
agccaagatggacaaa

SEQ ID NO: 320 - RNA, 16 nt, synthetic construct
  source 1..16: mol_type = other RNA
ttgctgaccacgtgga

SEQ ID NO: 321 - RNA, 16 nt, synthetic construct
  source 1..16: mol_type = other RNA
tgaactgcatctgcca

SEQ ID NO: 322 - RNA, 16 nt, synthetic construct
  source 1..16: mol_type = other RNA
gaactgcatctgccca

SEQ ID NO: 323 - RNA, 16 nt, synthetic construct
  source 1..16: mol_type = other RNA
aactgcatctgcccga

SEQ ID NO: 324 - RNA, 16 nt, synthetic construct
  source 1..16: mol_type = other RNA
actgcatctgcccgga

SEQ ID NO: 325 - RNA, 16 nt, synthetic construct
  source 1..16: mol_type = other RNA
ctgcatctgcccggca

SEQ ID NO: 326 - RNA, 16 nt, synthetic construct
  source 1..16: mol_type = other RNA
caccctcatcacccta

SEQ ID NO: 327 - RNA, 16 nt, synthetic construct
  source 1..16: mol_type = other RNA
accctcatcaccctga

SEQ ID NO: 328 - RNA, 16 nt, synthetic construct
  source 1..16: mol_type = other RNA
ccctcatcaccctgca

SEQ ID NO: 329 - RNA, 16 nt, synthetic construct
  source 1..16: mol_type = other RNA
tcaccctgctggtgca

SEQ ID NO: 330 - RNA, 16 nt, synthetic construct
  source 1..16: mol_type = other RNA
gctggtgcacacgtca

SEQ ID NO: 331 - RNA, 16 nt, synthetic construct
  source 1..16: mol_type = other RNA
agcagtggcagcgcaa

SEQ ID NO: 332 - RNA, 16 nt, synthetic construct
  source 1..16: mol_type = other RNA
agggcaagagcttcaa

SEQ ID NO: 333 - RNA, 16 nt, synthetic construct
  source 1..16: mol_type = other RNA
caagagcttcacctaa

SEQ ID NO: 334 - RNA, 16 nt, synthetic construct
  source 1..16: mol_type = other RNA
aagagcttcacctaca

SEQ ID NO: 335 - RNA, 16 nt, synthetic construct
  source 1..16: mol_type = other RNA
agagcttcacctacga

SEQ ID NO: 336 - RNA, 16 nt, synthetic construct
  source 1..16: mol_type = other RNA
gagcttcacctacgca

SEQ ID NO: 337 - RNA, 16 nt, synthetic construct
  source 1..16: mol_type = other RNA
tgtgcctcatcgggca

SEQ ID NO: 338 - RNA, 16 nt, synthetic construct
  source 1..16: mol_type = other RNA
tgcctcatcgggctga

SEQ ID NO: 339 - RNA, 16 nt, synthetic construct
  source 1..16: mol_type = other RNA
gcctcatcgggctgaa

SEQ ID NO: 340 - RNA, 16 nt, synthetic construct
  source 1..16: mol_type = other RNA
cctcatcgggctgaaa

SEQ ID NO: 341 - RNA, 16 nt, synthetic construct
  source 1..16: mol_type = other RNA
tcatcgggctgaagca

SEQ ID NO: 342 - RNA, 16 nt, synthetic construct
  source 1..16: mol_type = other RNA
catcgggctgaagcga

SEQ ID NO: 343 - RNA, 16 nt, synthetic construct
  source 1..16: mol_type = other RNA
ccaaggaggagaacta

SEQ ID NO: 344 - RNA, 16 nt, synthetic construct
  source 1..16: mol_type = other RNA
caaggaggagaactca

SEQ ID NO: 345 - RNA, 16 nt, synthetic construct
  source 1..16: mol_type = other RNA
ccacctcctgcctgta

SEQ ID NO: 346 - RNA, 16 nt, synthetic construct
  source 1..16: mol_type = other RNA
cacctcctgcctgtga

SEQ ID NO: 347 - RNA, 16 nt, synthetic construct
  source 1..16: mol_type = other RNA
acctcctgcctgtgaa

SEQ ID NO: 348 - RNA, 16 nt, synthetic construct
  source 1..16: mol_type = other RNA
cctcctgcctgtgaaa

SEQ ID NO: 349 - RNA, 16 nt, synthetic construct
  source 1..16: mol_type = other RNA
ggacaagggctgcaaa

SEQ ID NO: 350 - RNA, 16 nt, synthetic construct
  source 1..16: mol_type = other RNA
ggttcaagaacaagca

SEQ ID NO: 351 - RNA, 16 nt, synthetic construct
  source 1..16: mol_type = other RNA
gttcaagaacaagcta

SEQ ID NO: 352 - RNA, 16 nt, synthetic construct
  source 1..16: mol_type = other RNA
ttcaagaacaagctga

SEQ ID NO: 353 - RNA, 16 nt, synthetic construct
  source 1..16: mol_type = other RNA
tcaagaacaagctgaa

SEQ ID NO: 354 - RNA, 16 nt, synthetic construct
  source 1..16: mol_type = other RNA
caagaacaagctgata

SEQ ID NO: 355 - RNA, 16 nt, synthetic construct
  source 1..16: mol_type = other RNA
acaacttcatcgtgca

SEQ ID NO: 356 - RNA, 16 nt, synthetic construct
  source 1..16: mol_type = other RNA
accaccacttcctgga

SEQ ID NO: 357 - RNA, 16 nt, synthetic construct
  source 1..16: mol_type = other RNA
ccaccacttcctggaa

SEQ ID NO: 358 - RNA, 16 nt, synthetic construct
  source 1..16: mol_type = other RNA
ggtctactacatggaa

SEQ ID NO: 359 - RNA, 16 nt, synthetic construct
  source 1..16: mol_type = other RNA
gtctactacatggaga

SEQ ID NO: 360 - RNA, 16 nt, synthetic construct
  source 1..16: mol_type = other RNA
tctactacatggagga

SEQ ID NO: 361 - RNA, 16 nt, synthetic construct
  source 1..16: mol_type = other RNA
tggacaacctggacaa

SEQ ID NO: 362 - RNA, 16 nt, synthetic construct
  source 1..16: mol_type = other RNA
gacaacctggacagca

SEQ ID NO: 363 - RNA, 16 nt, synthetic construct
  source 1..16: mol_type = other RNA
tggtggtggacaagga

SEQ ID NO: 364 - RNA, 16 nt, synthetic construct
  source 1..16: mol_type = other RNA
ggtggtggacaaggaa

SEQ ID NO: 365 - RNA, 16 nt, synthetic construct
  source 1..16: mol_type = other RNA
gtggtggacaaggaga

SEQ ID NO: 366 - RNA, 16 nt, synthetic construct
  source 1..16: mol_type = other RNA
gtggacaaggagagca

SEQ ID NO: 367 - RNA, 16 nt, synthetic construct
  source 1..16: mol_type = other RNA
tggacaaggagagcaa

SEQ ID NO: 368 - RNA, 16 nt, synthetic construct
  source 1..16: mol_type = other RNA
ggacaaggagagcaca

SEQ ID NO: 369 - RNA, 16 nt, synthetic construct
  source 1..16: mol_type = other RNA
ccaagaccatcgtcaa

SEQ ID NO: 370 - RNA, 16 nt, synthetic construct
  source 1..16: mol_type = other RNA
caagaccatcgtcaaa

SEQ ID NO: 371 - RNA, 16 nt, synthetic construct
  source 1..16: mol_type = other RNA
tcagcatcaccacgga

SEQ ID NO: 372 - RNA, 16 nt, synthetic construct
  source 1..16: mol_type = other RNA
agcatcaccacggaga

SEQ ID NO: 373 - RNA, 16 nt, synthetic construct
  source 1..16: mol_type = other RNA
gcatcaccacggagca

SEQ ID NO: 374 - RNA, 16 nt, synthetic construct
  source 1..16: mol_type = other RNA
atcaccacggagctca

SEQ ID NO: 375 - RNA, 16 nt, synthetic construct
  source 1..16: mol_type = other RNA
tcaccacggagctcaa

SEQ ID NO: 376 - RNA, 16 nt, synthetic construct
  source 1..16: mol_type = other RNA
caccacggagctcaca

SEQ ID NO: 377 - RNA, 16 nt, synthetic construct
  source 1..16: mol_type = other RNA
acccttccaacatgca

SEQ ID NO: 378 - RNA, 16 nt, synthetic construct
  source 1..16: mol_type = other RNA
cccttccaacatgcga

SEQ ID NO: 379 - RNA, 16 nt, synthetic construct
  source 1..16: mol_type = other RNA
ccaaggacagctacta

SEQ ID NO: 380 - RNA, 16 nt, synthetic construct
  source 1..16: mol_type = other RNA
caaggacagctactca

SEQ ID NO: 381 - RNA, 16 nt, synthetic construct
  source 1..16: mol_type = other RNA
aaggacagctactcta

SEQ ID NO: 382 - RNA, 16 nt, synthetic construct
  source 1..16: mol_type = other RNA
aggacagctactctca

SEQ ID NO: 383 - RNA, 16 nt, synthetic construct
  source 1..16: mol_type = other RNA
ggacagctactctcta

SEQ ID NO: 384 - RNA, 16 nt, synthetic construct
  source 1..16: mol_type = other RNA
gacagctactctctga

SEQ ID NO: 385 - RNA, 16 nt, synthetic construct
  source 1..16: mol_type = other RNA
acagctactctctgga

SEQ ID NO: 386 - RNA, 16 nt, synthetic construct
  source 1..16: mol_type = other RNA
cagctactctctggca

SEQ ID NO: 387 - RNA, 16 nt, synthetic construct
  source 1..16: mol_type = other RNA
agctactctctggcta

SEQ ID NO: 388 - RNA, 16 nt, synthetic construct
  source 1..16: mol_type = other RNA
ctctctggctctttca

SEQ ID NO: 389 - RNA, 16 nt, synthetic construct
  source 1..16: mol_type = other RNA
tctctggctctttcca

SEQ ID NO: 390 - RNA, 16 nt, synthetic construct
  source 1..16: mol_type = other RNA
ctctggctctttccaa

SEQ ID NO: 391 - RNA, 16 nt, synthetic construct
  source 1..16: mol_type = other RNA
tctggctctttccaaa

SEQ ID NO: 392 - RNA, 16 nt, synthetic construct
  source 1..16: mol_type = other RNA
ctggctctttccaaaa

SEQ ID NO: 393 - RNA, 16 nt, synthetic construct
  source 1..16: mol_type = other RNA
tggctctttccaaaca

SEQ ID NO: 394 - RNA, 16 nt, synthetic construct
  source 1..16: mol_type = other RNA
ggctctttccaaacta

SEQ ID NO: 395 - RNA, 16 nt, synthetic construct
  source 1..16: mol_type = other RNA
gctccaacctggccta

SEQ ID NO: 396 - RNA, 16 nt, synthetic construct
  source 1..16: mol_type = other RNA
tccaacctggccttca

SEQ ID NO: 397 - RNA, 16 nt, synthetic construct
  source 1..16: mol_type = other RNA
acctggccttcatgta

SEQ ID NO: 398 - RNA, 16 nt, synthetic construct
  source 1..16: mol_type = other RNA
cctggccttcatgtta

SEQ ID NO: 399 - RNA, 16 nt, synthetic construct
  source 1..16: mol_type = other RNA
ctggccttcatgttca

SEQ ID NO: 400 - RNA, 16 nt, synthetic construct
  source 1..16: mol_type = other RNA
tggccttcatgttcca

SEQ ID NO: 401 - RNA, 16 nt, synthetic construct
  source 1..16: mol_type = other RNA
gccttcatgttccgca

SEQ ID NO: 402 - RNA, 16 nt, synthetic construct
  source 1..16: mol_type = other RNA
tcatgttccgcctgca

SEQ ID NO: 403 - RNA, 16 nt, synthetic construct
  source 1..16: mol_type = other RNA
catgttccgcctgcca

SEQ ID NO: 404 - RNA, 16 nt, synthetic construct
  source 1..16: mol_type = other RNA
ctctaccagtccttca

SEQ ID NO: 405 - RNA, 16 nt, synthetic construct
  source 1..16: mol_type = other RNA
tctaccagtccttcga

SEQ ID NO: 406 - RNA, 16 nt, synthetic construct
  source 1..16: mol_type = other RNA
ctaccagtccttcgta

SEQ ID NO: 407 - RNA, 16 nt, synthetic construct
  source 1..16: mol_type = other RNA
taccagtccttcgtga

SEQ ID NO: 408 - RNA, 16 nt, synthetic construct
  source 1..16: mol_type = other RNA
accagtccttcgtgaa

SEQ ID NO: 409 - RNA, 16 nt, synthetic construct
  source 1..16: mol_type = other RNA
ccagtccttcgtgaaa

SEQ ID NO: 410 - RNA, 16 nt, synthetic construct
  source 1..16: mol_type = other RNA
cagtccttcgtgaaga

SEQ ID NO: 411 - RNA, 16 nt, synthetic construct
  source 1..16: mol_type = other RNA
agtccttcgtgaagga

SEQ ID NO: 412 - RNA, 16 nt, synthetic construct
  source 1..16: mol_type = other RNA
cgtgaaggactacata

SEQ ID NO: 413 - RNA, 16 nt, synthetic construct
  source 1..16: mol_type = other RNA
gtgaaggactacatga

SEQ ID NO: 414 - RNA, 16 nt, synthetic construct
  source 1..16: mol_type = other RNA
tgaaggactacatgaa

SEQ ID NO: 415 - RNA, 16 nt, synthetic construct
  source 1..16: mol_type = other RNA
gaaggactacatgata

SEQ ID NO: 416 - RNA, 16 nt, synthetic construct
  source 1..16: mol_type = other RNA
aaggactacatgatca

SEQ ID NO: 417 - RNA, 16 nt, synthetic construct
  source 1..16: mol_type = other RNA
aggactacatgatcaa

SEQ ID NO: 418 - RNA, 16 nt, synthetic construct
  source 1..16: mol_type = other RNA
ggactacatgatcaca

SEQ ID NO: 419 - RNA, 16 nt, synthetic construct
  source 1..16: mol_type = other RNA
gactacatgatcacca

SEQ ID NO: 420 - RNA, 16 nt, synthetic construct
  source 1..16: mol_type = other RNA
actacatgatcaccaa

SEQ ID NO: 421 - RNA, 16 nt, synthetic construct
  source 1..16: mol_type = other RNA
tacatgatcaccatca

SEQ ID NO: 422 - RNA, 16 nt, synthetic construct
  source 1..16: mol_type = other RNA
acatgatcaccatcaa

SEQ ID NO: 423 - RNA, 16 nt, synthetic construct
  source 1..16: mol_type = other RNA
catgatcaccatcaca

SEQ ID NO: 424 - RNA, 16 nt, synthetic construct
  source 1..16: mol_type = other RNA
atgatcaccatcacca

SEQ ID NO: 425 - RNA, 16 nt, synthetic construct
  source 1..16: mol_type = other RNA
gacctgtggatccgca

SEQ ID NO: 426 - RNA, 16 nt, synthetic construct
  source 1..16: mol_type = other RNA
acggccgcctcttcca

SEQ ID NO: 427 - RNA, 16 nt, synthetic construct
  source 1..16: mol_type = other RNA
ggccgcctcttccaga

SEQ ID NO: 428 - RNA, 16 nt, synthetic construct
  source 1..16: mol_type = other RNA
gccgcctcttccagaa

SEQ ID NO: 429 - RNA, 16 nt, synthetic construct
  source 1..16: mol_type = other RNA
ccgcctcttccagaaa

SEQ ID NO: 430 - RNA, 16 nt, synthetic construct
  source 1..16: mol_type = other RNA
tgctcctccagcgcca

SEQ ID NO: 431 - RNA, 16 nt, synthetic construct
  source 1..16: mol_type = other RNA
tcctccagcgccgaga

SEQ ID NO: 432 - RNA, 16 nt, synthetic construct
  source 1..16: mol_type = other RNA
ctccagcgccgagata

SEQ ID NO: 433 - RNA, 16 nt, synthetic construct
  source 1..16: mol_type = other RNA
tccagcgccgagatca

SEQ ID NO: 434 - RNA, 16 nt, synthetic construct
  source 1..16: mol_type = other RNA
ccagcgccgagatcca

SEQ ID NO: 435 - RNA, 16 nt, synthetic construct
  source 1..16: mol_type = other RNA
cagcgccgagatccca

SEQ ID NO: 436 - RNA, 16 nt, synthetic construct
  source 1..16: mol_type = other RNA
ggccagcagctcccaa

SEQ ID NO: 437 - RNA, 21 nt, Homo sapiens
  source 1..21: mol_type = mRNA
caaggagcggctcaagctgtt

SEQ ID NO: 438 - RNA, 21 nt, Homo sapiens
  source 1..21: mol_type = mRNA
aaggagcggctcaagctgttc

SEQ ID NO: 439 - RNA, 21 nt, Homo sapiens
  source 1..21: mol_type = mRNA
ggagcggctcaagctgttctt

SEQ ID NO: 440 - RNA, 21 nt, Homo sapiens
  source 1..21: mol_type = mRNA
gagcggctcaagctgttcttc

SEQ ID NO: 441 - RNA, 21 nt, Homo sapiens
  source 1..21: mol_type = mRNA
agcggctcaagctgttcttca

SEQ ID NO: 442 - RNA, 21 nt, Homo sapiens
  source 1..21: mol_type = mRNA
gcggctcaagctgttcttcat

SEQ ID NO: 443 - RNA, 21 nt, Homo sapiens
  source 1..21: mol_type = mRNA
cggctcaagctgttcttcatc

SEQ ID NO: 444 - RNA, 21 nt, Homo sapiens
  source 1..21: mol_type = mRNA
ggctcaagctgttcttcatca

SEQ ID NO: 445 - RNA, 21 nt, Homo sapiens
  source 1..21: mol_type = mRNA
gctcaagctgttcttcatcaa

SEQ ID NO: 446 - RNA, 21 nt, Homo sapiens
  source 1..21: mol_type = mRNA
ctcaagctgttcttcatcaaa

SEQ ID NO: 447 - RNA, 21 nt, Homo sapiens
  source 1..21: mol_type = mRNA
tcaagctgttcttcatcaaaa

SEQ ID NO: 448 - RNA, 21 nt, Homo sapiens
  source 1..21: mol_type = mRNA
cctgaggatccggctgttcaa

SEQ ID NO: 449 - RNA, 21 nt, Homo sapiens
  source 1..21: mol_type = mRNA
ctgaggatccggctgttcaac

SEQ ID NO: 450 - RNA, 21 nt, Homo sapiens
  source 1..21: mol_type = mRNA
tgaggatccggctgttcaact

SEQ ID NO: 451 - RNA, 21 nt, Homo sapiens
  source 1..21: mol_type = mRNA
gaggatccggctgttcaactt

SEQ ID NO: 452 - RNA, 21 nt, Homo sapiens
  source 1..21: mol_type = mRNA
aggatccggctgttcaacttc

SEQ ID NO: 453 - RNA, 21 nt, Homo sapiens
  source 1..21: mol_type = mRNA
ggatccggctgttcaacttct

SEQ ID NO: 454 - RNA, 21 nt, Homo sapiens
  source 1..21: mol_type = mRNA
gatccggctgttcaacttctc

SEQ ID NO: 455 - RNA, 21 nt, Homo sapiens
  source 1..21: mol_type = mRNA
atccggctgttcaacttctcc

SEQ ID NO: 456 - RNA, 21 nt, Homo sapiens
  source 1..21: mol_type = mRNA
tccggctgttcaacttctccc

SEQ ID NO: 457 - RNA, 21 nt, Homo sapiens
  source 1..21: mol_type = mRNA
ccggctgttcaacttctccct

SEQ ID NO: 458 - RNA, 21 nt, Homo sapiens
  source 1..21: mol_type = mRNA
cggctgttcaacttctccctg

SEQ ID NO: 459 - RNA, 21 nt, Homo sapiens
  source 1..21: mol_type = mRNA
ccctgggcatcggatgctggg

SEQ ID NO: 460 - RNA, 21 nt, Homo sapiens
  source 1..21: mol_type = mRNA
cctgggcatcggatgctgggg

SEQ ID NO: 461 - RNA, 21 nt, Homo sapiens
  source 1..21: mol_type = mRNA
catctacctcagctacaaagg

SEQ ID NO: 462 - RNA, 21 nt, Homo sapiens
  source 1..21: mol_type = mRNA
atctacctcagctacaaaggc

SEQ ID NO: 463 - RNA, 21 nt, Homo sapiens
  source 1..21: mol_type = mRNA
acctcagctacaaaggcaaca

SEQ ID NO: 464 - RNA, 21 nt, Homo sapiens
  source 1..21: mol_type = mRNA
cctcagctacaaaggcaacat

SEQ ID NO: 465 - RNA, 21 nt, Homo sapiens
  source 1..21: mol_type = mRNA
ctcagctacaaaggcaacatc

SEQ ID NO: 466 - RNA, 21 nt, Homo sapiens
  source 1..21: mol_type = mRNA
tcagctacaaaggcaacatct

SEQ ID NO: 467 - RNA, 21 nt, Homo sapiens
  source 1..21: mol_type = mRNA
cagctacaaaggcaacatctg

SEQ ID NO: 468 - RNA, 21 nt, Homo sapiens
  source 1..21: mol_type = mRNA
agctacaaaggcaacatctgg

SEQ ID NO: 469 - RNA, 21 nt, Homo sapiens
  source 1..21: mol_type = mRNA
gctacaaaggcaacatctggg

SEQ ID NO: 470 - RNA, 21 nt, Homo sapiens
  source 1..21: mol_type = mRNA
ctacaaaggcaacatctggga

SEQ ID NO: 471 - RNA, 21 nt, Homo sapiens
  source 1..21: mol_type = mRNA
tacaaaggcaacatctgggag

SEQ ID NO: 472 - RNA, 21 nt, Homo sapiens
  source 1..21: mol_type = mRNA
acaaaggcaacatctgggagc

SEQ ID NO: 473 - RNA, 21 nt, Homo sapiens
  source 1..21: mol_type = mRNA
caaaggcaacatctgggagca

SEQ ID NO: 474 - RNA, 21 nt, Homo sapiens
  source 1..21: mol_type = mRNA
gtcctggagatgatcaacact

SEQ ID NO: 475 - RNA, 21 nt, Homo sapiens
  source 1..21: mol_type = mRNA
cactctgcccttcatcatcac

SEQ ID NO: 476 - RNA, 21 nt, Homo sapiens
  source 1..21: mol_type = mRNA
actctgcccttcatcatcacg

SEQ ID NO: 477 - RNA, 21 nt, Homo sapiens
  source 1..21: mol_type = mRNA
ctctgcccttcatcatcacga

SEQ ID NO: 478 - RNA, 21 nt, Homo sapiens
  source 1..21: mol_type = mRNA
gccgctgcggaacctgttcat

SEQ ID NO: 479 - RNA, 21 nt, Homo sapiens
  source 1..21: mol_type = mRNA
ccgctgcggaacctgttcatc

SEQ ID NO: 480 - RNA, 21 nt, Homo sapiens
  source 1..21: mol_type = mRNA
cgctgcggaacctgttcatcc

SEQ ID NO: 481 - RNA, 21 nt, Homo sapiens
  source 1..21: mol_type = mRNA
gctgcggaacctgttcatccc

SEQ ID NO: 482 - RNA, 21 nt, Homo sapiens
  source 1..21: mol_type = mRNA
ctgcggaacctgttcatcccc

SEQ ID NO: 483 - RNA, 21 nt, Homo sapiens
  source 1..21: mol_type = mRNA
taatgacttccaccgtgccat

SEQ ID NO: 484 - RNA, 21 nt, Homo sapiens
  source 1..21: mol_type = mRNA
aatgacttccaccgtgccatc

SEQ ID NO: 485 - RNA, 21 nt, Homo sapiens
  source 1..21: mol_type = mRNA
atgacttccaccgtgccatcc

SEQ ID NO: 486 - RNA, 21 nt, Homo sapiens
  source 1..21: mol_type = mRNA
tgacttccaccgtgccatcct

SEQ ID NO: 487 - RNA, 21 nt, Homo sapiens
  source 1..21: mol_type = mRNA
gacttccaccgtgccatcctg

SEQ ID NO: 488 - RNA, 21 nt, Homo sapiens
  source 1..21: mol_type = mRNA
gtcagccatgttcaaccaggt

SEQ ID NO: 489 - RNA, 21 nt, Homo sapiens
  source 1..21: mol_type = mRNA
cctcttctgcaccctgctgtg

SEQ ID NO: 490 - RNA, 21 nt, Homo sapiens
  source 1..21: mol_type = mRNA
cttctgcaccctgctgtgcct

SEQ ID NO: 491 - RNA, 21 nt, Homo sapiens
  source 1..21: mol_type = mRNA
cctcctgacctccttctactt

SEQ ID NO: 492 - RNA, 21 nt, Homo sapiens
  source 1..21: mol_type = mRNA
tcctgacctccttctacttct

SEQ ID NO: 493 - RNA, 21 nt, Homo sapiens
  source 1..21: mol_type = mRNA
cctgacctccttctacttctg

SEQ ID NO: 494 - RNA, 21 nt, Homo sapiens
  source 1..21: mol_type = mRNA
tgacctccttctacttctgca

SEQ ID NO: 495 - RNA, 21 nt, Homo sapiens
  source 1..21: mol_type = mRNA
gacctccttctacttctgcat

SEQ ID NO: 496 - RNA, 21 nt, Homo sapiens
  source 1..21: mol_type = mRNA
acctccttctacttctgcatc

SEQ ID NO: 497 - RNA, 21 nt, Homo sapiens
  source 1..21: mol_type = mRNA
cctccttctacttctgcatcg

SEQ ID NO: 498 - RNA, 21 nt, Homo sapiens
  source 1..21: mol_type = mRNA
ctccttctacttctgcatcgt

SEQ ID NO: 499 - RNA, 21 nt, Homo sapiens
  source 1..21: mol_type = mRNA
tccttctacttctgcatcgtc

SEQ ID NO: 500 - RNA, 21 nt, Homo sapiens
  source 1..21: mol_type = mRNA
cgtcacgcccaagatctggcc

SEQ ID NO: 501 - RNA, 21 nt, Homo sapiens
  source 1..21: mol_type = mRNA
cctcgtggtgctcccactgca

SEQ ID NO: 502 - RNA, 21 nt, Homo sapiens
  source 1..21: mol_type = mRNA
ctcgtggtgctcccactgcag

SEQ ID NO: 503 - RNA, 21 nt, Homo sapiens
  source 1..21: mol_type = mRNA
gctcgtctacctctggatgga

SEQ ID NO: 504 - RNA, 21 nt, Homo sapiens
  source 1..21: mol_type = mRNA
ctcgtctacctctggatggag

SEQ ID NO: 505 - RNA, 21 nt, Homo sapiens
  source 1..21: mol_type = mRNA
tcgtctacctctggatggagc

SEQ ID NO: 506 - RNA, 21 nt, Homo sapiens
  source 1..21: mol_type = mRNA
cgtctacctctggatggagcg

SEQ ID NO: 507 - RNA, 21 nt, Homo sapiens
  source 1..21: mol_type = mRNA
ccttctcatggacttcctgaa

SEQ ID NO: 508 - RNA, 21 nt, Homo sapiens
  source 1..21: mol_type = mRNA
cttctcatggacttcctgaac

SEQ ID NO: 509 - RNA, 21 nt, Homo sapiens
  source 1..21: mol_type = mRNA
ctattacgtggtcatcctgtg

SEQ ID NO: 510 - RNA, 21 nt, Homo sapiens
  source 1..21: mol_type = mRNA
ctctgtggtcccagcgggtca

SEQ ID NO: 511 - RNA, 21 nt, Homo sapiens
  source 1..21: mol_type = mRNA
tctgtggtcccagcgggtcat

SEQ ID NO: 512 - RNA, 21 nt, Homo sapiens
  source 1..21: mol_type = mRNA
ctgtggtcccagcgggtcatc

SEQ ID NO: 513 - RNA, 21 nt, Homo sapiens
  source 1..21: mol_type = mRNA
tgtggtcccagcgggtcatct

SEQ ID NO: 514 - RNA, 21 nt, Homo sapiens
  source 1..21: mol_type = mRNA
gtggtcccagcgggtcatcta

SEQ ID NO: 515 - RNA, 21 nt, Homo sapiens
  source 1..21: mol_type = mRNA
tggtcccagcgggtcatctac

SEQ ID NO: 516 - RNA, 21 nt, Homo sapiens
  source 1..21: mol_type = mRNA
ggtcccagcgggtcatctacc

SEQ ID NO: 517 - RNA, 21 nt, Homo sapiens
  source 1..21: mol_type = mRNA
gtcccagcgggtcatctacct

SEQ ID NO: 518 - RNA, 21 nt, Homo sapiens
  source 1..21: mol_type = mRNA
tcccagcgggtcatctacctc

SEQ ID NO: 519 - RNA, 21 nt, Homo sapiens
  source 1..21: mol_type = mRNA
cccagcgggtcatctacctcc

SEQ ID NO: 520 - RNA, 21 nt, Homo sapiens
  source 1..21: mol_type = mRNA
ccagcgggtcatctacctcca

SEQ ID NO: 521 - RNA, 21 nt, Homo sapiens
  source 1..21: mol_type = mRNA
cagcgggtcatctacctccag

SEQ ID NO: 522 - RNA, 21 nt, Homo sapiens
  source 1..21: mol_type = mRNA
agcgggtcatctacctccagg

SEQ ID NO: 523 - RNA, 21 nt, Homo sapiens
  source 1..21: mol_type = mRNA
gcgggtcatctacctccaggg

SEQ ID NO: 524 - RNA, 21 nt, Homo sapiens
  source 1..21: mol_type = mRNA
gggtcatctacctccagggct

SEQ ID NO: 525 - RNA, 21 nt, Homo sapiens
  source 1..21: mol_type = mRNA
ggtcatctacctccagggctc

SEQ ID NO: 526 - RNA, 21 nt, Homo sapiens
  source 1..21: mol_type = mRNA
gtcatctacctccagggctct

SEQ ID NO: 527 - RNA, 21 nt, Homo sapiens
  source 1..21: mol_type = mRNA
tcatctacctccagggctctg

SEQ ID NO: 528 - RNA, 21 nt, Homo sapiens
  source 1..21: mol_type = mRNA
catctacctccagggctctgc

SEQ ID NO: 529 - RNA, 21 nt, Homo sapiens
  source 1..21: mol_type = mRNA
atctacctccagggctctgca

SEQ ID NO: 530 - RNA, 21 nt, Homo sapiens
  source 1..21: mol_type = mRNA
ggacctcatgcgagccaagat

SEQ ID NO: 531 - RNA, 21 nt, Homo sapiens
  source 1..21: mol_type = mRNA
gacctcatgcgagccaagatg

SEQ ID NO: 532 - RNA, 21 nt, Homo sapiens
  source 1..21: mol_type = mRNA
acctcatgcgagccaagatgg

SEQ ID NO: 533 - RNA, 21 nt, Homo sapiens
  source 1..21: mol_type = mRNA
cctcatgcgagccaagatgga

SEQ ID NO: 534 - RNA, 21 nt, Homo sapiens
  source 1..21: mol_type = mRNA
ctcatgcgagccaagatggac

SEQ ID NO: 535 - RNA, 21 nt, Homo sapiens
  source 1..21: mol_type = mRNA
tcatgcgagccaagatggaca

SEQ ID NO: 536 - RNA, 21 nt, Homo sapiens
  source 1..21: mol_type = mRNA
catgcgagccaagatggacaa

SEQ ID NO: 537 - RNA, 21 nt, Homo sapiens
  source 1..21: mol_type = mRNA
atgcgagccaagatggacaat

SEQ ID NO: 538 - RNA, 21 nt, Homo sapiens
  source 1..21: mol_type = mRNA
caagtttgctgaccacgtggt

SEQ ID NO: 539 - RNA, 21 nt, Homo sapiens
  source 1..21: mol_type = mRNA
ggcgctgaactgcatctgccc

SEQ ID NO: 540 - RNA, 21 nt, Homo sapiens
  source 1..21: mol_type = mRNA
gcgctgaactgcatctgcccg

SEQ ID NO: 541 - RNA, 21 nt, Homo sapiens
  source 1..21: mol_type = mRNA
cgctgaactgcatctgcccgg

SEQ ID NO: 542 - RNA, 21 nt, Homo sapiens
  source 1..21: mol_type = mRNA
gctgaactgcatctgcccggc

SEQ ID NO: 543 - RNA, 21 nt, Homo sapiens
  source 1..21: mol_type = mRNA
ctgaactgcatctgcccggcg

SEQ ID NO: 544 - RNA, 21 nt, Homo sapiens
  source 1..21: mol_type = mRNA
acctccaccctcatcaccctg

SEQ ID NO: 545 - RNA, 21 nt, Homo sapiens
  source 1..21: mol_type = mRNA
cctccaccctcatcaccctgc

SEQ ID NO: 546 - RNA, 21 nt, Homo sapiens
  source 1..21: mol_type = mRNA
ctccaccctcatcaccctgct

SEQ ID NO: 547 - RNA, 21 nt, Homo sapiens
  source 1..21: mol_type = mRNA
cctcatcaccctgctggtgca

SEQ ID NO: 548 - RNA, 21 nt, Homo sapiens
  source 1..21: mol_type = mRNA
accctgctggtgcacacgtcc

SEQ ID NO: 549 - RNA, 21 nt, Homo sapiens
  source 1..21: mol_type = mRNA
tccggagcagtggcagcgcat

SEQ ID NO: 550 - RNA, 21 nt, Homo sapiens
  source 1..21: mol_type = mRNA
gtacgagggcaagagcttcac

SEQ ID NO: 551 - RNA, 21 nt, Homo sapiens
  source 1..21: mol_type = mRNA
gagggcaagagcttcacctac

SEQ ID NO: 552 - RNA, 21 nt, Homo sapiens
  source 1..21: mol_type = mRNA
agggcaagagcttcacctacg

SEQ ID NO: 553 - RNA, 21 nt, Homo sapiens
  source 1..21: mol_type = mRNA
gggcaagagcttcacctacgc

SEQ ID NO: 554 - RNA, 21 nt, Homo sapiens
  source 1..21: mol_type = mRNA
ggcaagagcttcacctacgcg

SEQ ID NO: 555 - RNA, 21 nt, Homo sapiens
  source 1..21: mol_type = mRNA
tggcgtgtgcctcatcgggct

SEQ ID NO: 556 - RNA, 21 nt, Homo sapiens
  source 1..21: mol_type = mRNA
gcgtgtgcctcatcgggctga

SEQ ID NO: 557 - RNA, 21 nt, Homo sapiens
  source 1..21: mol_type = mRNA
cgtgtgcctcatcgggctgaa

SEQ ID NO: 558 - RNA, 21 nt, Homo sapiens
  source 1..21: mol_type = mRNA
gtgtgcctcatcgggctgaag

SEQ ID NO: 559 - RNA, 21 nt, Homo sapiens
  source 1..21: mol_type = mRNA
gtgcctcatcgggctgaagcg

SEQ ID NO: 560 - RNA, 21 nt, Homo sapiens
  source 1..21: mol_type = mRNA
tgcctcatcgggctgaagcgg

SEQ ID NO: 561 - RNA, 21 nt, Homo sapiens
  source 1..21: mol_type = mRNA
catcaccaaggaggagaactc

SEQ ID NO: 562 - RNA, 21 nt, Homo sapiens
  source 1..21: mol_type = mRNA
atcaccaaggaggagaactcg

SEQ ID NO: 563 - RNA, 21 nt, Homo sapiens
  source 1..21: mol_type = mRNA
ctgtgccacctcctgcctgtg

SEQ ID NO: 564 - RNA, 21 nt, Homo sapiens
  source 1..21: mol_type = mRNA
tgtgccacctcctgcctgtga

SEQ ID NO: 565 - RNA, 21 nt, Homo sapiens
  source 1..21: mol_type = mRNA
gtgccacctcctgcctgtgaa

SEQ ID NO: 566 - RNA, 21 nt, Homo sapiens
  source 1..21: mol_type = mRNA
tgccacctcctgcctgtgaaa

SEQ ID NO: 567 - RNA, 21 nt, Homo sapiens
  source 1..21: mol_type = mRNA
cggctggacaagggctgcaag

SEQ ID NO: 568 - RNA, 21 nt, Homo sapiens
  source 1..21: mol_type = mRNA
ctacgggttcaagaacaagct

SEQ ID NO: 569 - RNA, 21 nt, Homo sapiens
  source 1..21: mol_type = mRNA
tacgggttcaagaacaagctg

SEQ ID NO: 570 - RNA, 21 nt, Homo sapiens
  source 1..21: mol_type = mRNA
acgggttcaagaacaagctga

SEQ ID NO: 571 - RNA, 21 nt, Homo sapiens
  source 1..21: mol_type = mRNA
cgggttcaagaacaagctgat

SEQ ID NO: 572 - RNA, 21 nt, Homo sapiens
  source 1..21: mol_type = mRNA
gggttcaagaacaagctgatc

SEQ ID NO: 573 - RNA, 21 nt, Homo sapiens
  source 1..21: mol_type = mRNA
gctgtacaacttcatcgtgcc

SEQ ID NO: 574 - RNA, 21 nt, Homo sapiens
  source 1..21: mol_type = mRNA
gcccgaccaccacttcctgga

SEQ ID NO: 575 - RNA, 21 nt, Homo sapiens
  source 1..21: mol_type = mRNA
cccgaccaccacttcctggaa

SEQ ID NO: 576 - RNA, 21 nt, Homo sapiens
  source 1..21: mol_type = mRNA
cccatggtctactacatggag

SEQ ID NO: 577 - RNA, 21 nt, Homo sapiens
  source 1..21: mol_type = mRNA
ccatggtctactacatggagg

SEQ ID NO: 578 - RNA, 21 nt, Homo sapiens
  source 1..21: mol_type = mRNA
catggtctactacatggaggg

SEQ ID NO: 579 - RNA, 21 nt, Homo sapiens
  source 1..21: mol_type = mRNA
ctctgtggacaacctggacag

SEQ ID NO: 580 - RNA, 21 nt, Homo sapiens
  source 1..21: mol_type = mRNA
ctgtggacaacctggacagcc

SEQ ID NO: 581 - RNA, 21 nt, Homo sapiens
  source 1..21: mol_type = mRNA
cctggtggtggtggacaagga

SEQ ID NO: 582 - RNA, 21 nt, Homo sapiens
  source 1..21: mol_type = mRNA
ctggtggtggtggacaaggag

SEQ ID NO: 583 - RNA, 21 nt, Homo sapiens
  source 1..21: mol_type = mRNA
tggtggtggtggacaaggaga

SEQ ID NO: 584 - RNA, 21 nt, Homo sapiens
  source 1..21: mol_type = mRNA
tggtggtggacaaggagagca

SEQ ID NO: 585 - RNA, 21 nt, Homo sapiens
  source 1..21: mol_type = mRNA
ggtggtggacaaggagagcac

SEQ ID NO: 586 - RNA, 21 nt, Homo sapiens
  source 1..21: mol_type = mRNA
gtggtggacaaggagagcacc

SEQ ID NO: 587 - RNA, 21 nt, Homo sapiens
  source 1..21: mol_type = mRNA
ggacgccaagaccatcgtcaa

SEQ ID NO: 588 - RNA, 21 nt, Homo sapiens
  source 1..21: mol_type = mRNA
gacgccaagaccatcgtcaac

SEQ ID NO: 589 - RNA, 21 nt, Homo sapiens
  source 1..21: mol_type = mRNA
cagcctcagcatcaccacgga

SEQ ID NO: 590 - RNA, 21 nt, Homo sapiens
  source 1..21: mol_type = mRNA
gcctcagcatcaccacggagc

SEQ ID NO: 591 - RNA, 21 nt, Homo sapiens
  source 1..21: mol_type = mRNA
cctcagcatcaccacggagct

SEQ ID NO: 592 - RNA, 21 nt, Homo sapiens
  source 1..21: mol_type = mRNA
tcagcatcaccacggagctca

SEQ ID NO: 593 - RNA, 21 nt, Homo sapiens
  source 1..21: mol_type = mRNA
cagcatcaccacggagctcac

SEQ ID NO: 594 - RNA, 21 nt, Homo sapiens
  source 1..21: mol_type = mRNA
agcatcaccacggagctcacc

SEQ ID NO: 595 - RNA, 21 nt, Homo sapiens
  source 1..21: mol_type = mRNA
cacccacccttccaacatgcg

SEQ ID NO: 596 - RNA, 21 nt, Homo sapiens
  source 1..21: mol_type = mRNA
acccacccttccaacatgcgc

SEQ ID NO: 597 - RNA, 21 nt, Homo sapiens
  source 1..21: mol_type = mRNA
ccgcgccaaggacagctactc

SEQ ID NO: 598 - RNA, 21 nt, Homo sapiens
  source 1..21: mol_type = mRNA
cgcgccaaggacagctactct

SEQ ID NO: 599 - RNA, 21 nt, Homo sapiens
  source 1..21: mol_type = mRNA
gcgccaaggacagctactctc

SEQ ID NO: 600 - RNA, 21 nt, Homo sapiens
  source 1..21: mol_type = mRNA
cgccaaggacagctactctct

SEQ ID NO: 601 - RNA, 21 nt, Homo sapiens
  source 1..21: mol_type = mRNA
gccaaggacagctactctctg

SEQ ID NO: 602 - RNA, 21 nt, Homo sapiens
  source 1..21: mol_type = mRNA
ccaaggacagctactctctgg

SEQ ID NO: 603 - RNA, 21 nt, Homo sapiens
  source 1..21: mol_type = mRNA
caaggacagctactctctggc

SEQ ID NO: 604 - RNA, 21 nt, Homo sapiens
  source 1..21: mol_type = mRNA
aaggacagctactctctggct

SEQ ID NO: 605 - RNA, 21 nt, Homo sapiens
  source 1..21: mol_type = mRNA
aggacagctactctctggctc

SEQ ID NO: 606 - RNA, 21 nt, Homo sapiens
  source 1..21: mol_type = mRNA
agctactctctggctctttcc

SEQ ID NO: 607 - RNA, 21 nt, Homo sapiens
  source 1..21: mol_type = mRNA
gctactctctggctctttcca

SEQ ID NO: 608 - RNA, 21 nt, Homo sapiens
  source 1..21: mol_type = mRNA
ctactctctggctctttccaa

SEQ ID NO: 609 - RNA, 21 nt, Homo sapiens
  source 1..21: mol_type = mRNA
tactctctggctctttccaaa

SEQ ID NO: 610 - RNA, 21 nt, Homo sapiens
  source 1..21: mol_type = mRNA
actctctggctctttccaaac

SEQ ID NO: 611 - RNA, 21 nt, Homo sapiens
  source 1..21: mol_type = mRNA
ctctctggctctttccaaact

SEQ ID NO: 612 - RNA, 21 nt, Homo sapiens
  source 1..21: mol_type = mRNA
tctctggctctttccaaacta

SEQ ID NO: 613 - RNA, 21 nt, Homo sapiens
  source 1..21: mol_type = mRNA
gaatggctccaacctggcctt

SEQ ID NO: 614 - RNA, 21 nt, Homo sapiens
  source 1..21: mol_type = mRNA
atggctccaacctggccttca

SEQ ID NO: 615 - RNA, 21 nt, Homo sapiens
  source 1..21: mol_type = mRNA
ctccaacctggccttcatgtt

SEQ ID NO: 616 - RNA, 21 nt, Homo sapiens
  source 1..21: mol_type = mRNA
tccaacctggccttcatgttc

SEQ ID NO: 617 - RNA, 21 nt, Homo sapiens
  source 1..21: mol_type = mRNA
ccaacctggccttcatgttcc

SEQ ID NO: 618 - RNA, 21 nt, Homo sapiens
  source 1..21: mol_type = mRNA
caacctggccttcatgttccg

SEQ ID NO: 619 - RNA, 21 nt, Homo sapiens
  source 1..21: mol_type = mRNA
acctggccttcatgttccgcc

SEQ ID NO: 620 - RNA, 21 nt, Homo sapiens
  source 1..21: mol_type = mRNA
ggccttcatgttccgcctgcc

SEQ ID NO: 621 - RNA, 21 nt, Homo sapiens
  source 1..21: mol_type = mRNA
gccttcatgttccgcctgccg

SEQ ID NO: 622 - RNA, 21 nt, Homo sapiens
  source 1..21: mol_type = mRNA
cactgctctaccagtccttcg

SEQ ID NO: 623 - RNA, 21 nt, Homo sapiens
  source 1..21: mol_type = mRNA
actgctctaccagtccttcgt

SEQ ID NO: 624 - RNA, 21 nt, Homo sapiens
  source 1..21: mol_type = mRNA
ctgctctaccagtccttcgtg

SEQ ID NO: 625 - RNA, 21 nt, Homo sapiens
  source 1..21: mol_type = mRNA
tgctctaccagtccttcgtga

SEQ ID NO: 626 - RNA, 21 nt, Homo sapiens
  source 1..21: mol_type = mRNA
gctctaccagtccttcgtgaa

SEQ ID NO: 627 - RNA, 21 nt, Homo sapiens
  source 1..21: mol_type = mRNA
ctctaccagtccttcgtgaag

SEQ ID NO: 628 - RNA, 21 nt, Homo sapiens
  source 1..21: mol_type = mRNA
tctaccagtccttcgtgaagg

SEQ ID NO: 629 - RNA, 21 nt, Homo sapiens
  source 1..21: mol_type = mRNA
ctaccagtccttcgtgaagga

SEQ ID NO: 630 - RNA, 21 nt, Homo sapiens
  source 1..21: mol_type = mRNA
tccttcgtgaaggactacatg

SEQ ID NO: 631 - RNA, 21 nt, Homo sapiens
  source 1..21: mol_type = mRNA
ccttcgtgaaggactacatga

SEQ ID NO: 632 - RNA, 21 nt, Homo sapiens
  source 1..21: mol_type = mRNA
cttcgtgaaggactacatgat

SEQ ID NO: 633 - RNA, 21 nt, Homo sapiens
  source 1..21: mol_type = mRNA
ttcgtgaaggactacatgatc

SEQ ID NO: 634 - RNA, 21 nt, Homo sapiens
  source 1..21: mol_type = mRNA
tcgtgaaggactacatgatca

SEQ ID NO: 635 - RNA, 21 nt, Homo sapiens
  source 1..21: mol_type = mRNA
cgtgaaggactacatgatcac

SEQ ID NO: 636 - RNA, 21 nt, Homo sapiens
  source 1..21: mol_type = mRNA
gtgaaggactacatgatcacc

SEQ ID NO: 637 - RNA, 21 nt, Homo sapiens
  source 1..21: mol_type = mRNA
tgaaggactacatgatcacca

SEQ ID NO: 638 - RNA, 21 nt, Homo sapiens
  source 1..21: mol_type = mRNA
gaaggactacatgatcaccat

SEQ ID NO: 639 - RNA, 21 nt, Homo sapiens
  source 1..21: mol_type = mRNA
aggactacatgatcaccatca

SEQ ID NO: 640 - RNA, 21 nt, Homo sapiens
  source 1..21: mol_type = mRNA
ggactacatgatcaccatcac

SEQ ID NO: 641 - RNA, 21 nt, Homo sapiens
  source 1..21: mol_type = mRNA
gactacatgatcaccatcacc

SEQ ID NO: 642 - RNA, 21 nt, Homo sapiens
  source 1..21: mol_type = mRNA
actacatgatcaccatcaccc

SEQ ID NO: 643 - RNA, 21 nt, Homo sapiens
  source 1..21: mol_type = mRNA
agggcgacctgtggatccgca

SEQ ID NO: 644 - RNA, 21 nt, Homo sapiens
  source 1..21: mol_type = mRNA
cacgtacggccgcctcttcca

SEQ ID NO: 645 - RNA, 21 nt, Homo sapiens
  source 1..21: mol_type = mRNA
cgtacggccgcctcttccaga

SEQ ID NO: 646 - RNA, 21 nt, Homo sapiens
  source 1..21: mol_type = mRNA
gtacggccgcctcttccagaa

SEQ ID NO: 647 - RNA, 21 nt, Homo sapiens
  source 1..21: mol_type = mRNA
tacggccgcctcttccagaag

SEQ ID NO: 648 - RNA, 21 nt, Homo sapiens
  source 1..21: mol_type = mRNA
agctctgctcctccagcgccg

SEQ ID NO: 649 - RNA, 21 nt, Homo sapiens
  source 1..21: mol_type = mRNA
tctgctcctccagcgccgaga

SEQ ID NO: 650 - RNA, 21 nt, Homo sapiens
  source 1..21: mol_type = mRNA
tgctcctccagcgccgagatc

SEQ ID NO: 651 - RNA, 21 nt, Homo sapiens
  source 1..21: mol_type = mRNA
gctcctccagcgccgagatcc

SEQ ID NO: 652 - RNA, 21 nt, Homo sapiens
  source 1..21: mol_type = mRNA
ctcctccagcgccgagatccc

SEQ ID NO: 653 - RNA, 21 nt, Homo sapiens
  source 1..21: mol_type = mRNA
tcctccagcgccgagatcccc

SEQ ID NO: 654 - RNA, 21 nt, Homo sapiens
  source 1..21: mol_type = mRNA
cacgtggccagcagctcccag

SEQ ID NO: 655 - RNA, 21 nt, synthetic construct
  source 1..21: mol_type = other RNA
tggcggtgtccaggagcgcgc

SEQ ID NO: 656 - RNA, 21 nt, synthetic construct
  source 1..21: mol_type = other RNA
tgcaagggcagcacctcggag

SEQ ID NO: 657 - RNA, 21 nt, synthetic construct
  source 1..21: mol_type = other RNA
tgtcgttctggaaggacgggt

SEQ ID NO: 658 - RNA, 21 nt, synthetic construct
  source 1..21: mol_type = other RNA
tgaactccacctggaccctgt

SEQ ID NO: 659 - RNA, 21 nt, synthetic construct
  source 1..21: mol_type = other RNA
tagaactccacctggaccctg

SEQ ID NO: 660 - RNA, 21 nt, synthetic construct
  source 1..21: mol_type = other RNA
ttagaactccacctggaccct

SEQ ID NO: 661 - RNA, 21 nt, synthetic construct
  source 1..21: mol_type = other RNA
ttgagccgctccttgaaggtg

SEQ ID NO: 662 - RNA, 21 nt, synthetic construct
  source 1..21: mol_type = other RNA
tcagccggatcctcaggctcg

SEQ ID NO: 663 - RNA, 21 nt, synthetic construct
  source 1..21: mol_type = other RNA
tacagccggatcctcaggctc

SEQ ID NO: 664 - RNA, 21 nt, synthetic construct
  source 1..21: mol_type = other RNA
taacagccggatcctcaggct

SEQ ID NO: 665 - RNA, 21 nt, synthetic construct
  source 1..21: mol_type = other RNA
tgaacagccggatcctcaggc

SEQ ID NO: 666 - RNA, 21 nt, synthetic construct
  source 1..21: mol_type = other RNA
tcagggagaagttgaacagcc

SEQ ID NO: 667 - RNA, 21 nt, synthetic construct
  source 1..21: mol_type = other RNA
tttcagggagaagttgaacag

SEQ ID NO: 668 - RNA, 21 nt, synthetic construct
  source 1..21: mol_type = other RNA
tcttcagggagaagttgaaca

SEQ ID NO: 669 - RNA, 21 nt, synthetic construct
  source 1..21: mol_type = other RNA
tgtagttctgctttgggcagc

SEQ ID NO: 670 - RNA, 21 nt, synthetic construct
  source 1..21: mol_type = other RNA
tgagtagttctgctttgggca

SEQ ID NO: 671 - RNA, 21 nt, synthetic construct
  source 1..21: mol_type = other RNA
tacgagtcattgaaggagtag

SEQ ID NO: 672 - RNA, 21 nt, synthetic construct
  source 1..21: mol_type = other RNA
tgaggacgagtcattgaagga

SEQ ID NO: 673 - RNA, 21 nt, synthetic construct
  source 1..21: mol_type = other RNA
tccagttgatctcggaggacg

SEQ ID NO: 674 - RNA, 21 nt, synthetic construct
  source 1..21: mol_type = other RNA
tcccagttgatctcggaggac

SEQ ID NO: 675 - RNA, 21 nt, synthetic construct
  source 1..21: mol_type = other RNA
tgagcccagttgatctcggag

SEQ ID NO: 676 - RNA, 21 nt, synthetic construct
  source 1..21: mol_type = other RNA
tgtcatctttctctccaccca

SEQ ID NO: 677 - RNA, 21 nt, synthetic construct
  source 1..21: mol_type = other RNA
tcagtgtcatctttctctcca

SEQ ID NO: 678 - RNA, 21 nt, synthetic construct
  source 1..21: mol_type = other RNA
tcacagtgtcatctttctctc

SEQ ID NO: 679 - RNA, 21 nt, synthetic construct
  source 1..21: mol_type = other RNA
tgccacgatgacctggatcgc

SEQ ID NO: 680 - RNA, 21 nt, synthetic construct
  source 1..21: mol_type = other RNA
tgaagcttattatggccacga

SEQ ID NO: 681 - RNA, 21 nt, synthetic construct
  source 1..21: mol_type = other RNA
tggaagcttattatggccacg

SEQ ID NO: 682 - RNA, 21 nt, synthetic construct
  source 1..21: mol_type = other RNA
tcaggaagcttattatggcca

SEQ ID NO: 683 - RNA, 21 nt, synthetic construct
  source 1..21: mol_type = other RNA
tgaggtagatgagaagcatcg

SEQ ID NO: 684 - RNA, 21 nt, synthetic construct
  source 1..21: mol_type = other RNA
ttgctcccagatgttgccttt

SEQ ID NO: 685 - RNA, 21 nt, synthetic construct
  source 1..21: mol_type = other RNA
tctgctcccagatgttgcctt

SEQ ID NO: 686 - RNA, 21 nt, synthetic construct
  source 1..21: mol_type = other RNA
tggacgaaggacacgcggaag

SEQ ID NO: 687 - RNA, 21 nt, synthetic construct
  source 1..21: mol_type = other RNA
tcaggacgaaggacacgcgga

SEQ ID NO: 688 - RNA, 21 nt, synthetic construct
  source 1..21: mol_type = other RNA
ttgatcatctccaggacgaag

SEQ ID NO: 689 - RNA, 21 nt, synthetic construct
  source 1..21: mol_type = other RNA
tttgatcatctccaggacgaa

SEQ ID NO: 690 - RNA, 21 nt, synthetic construct
  source 1..21: mol_type = other RNA
tgttgatcatctccaggacga

SEQ ID NO: 691 - RNA, 21 nt, synthetic construct
  source 1..21: mol_type = other RNA
ttgttgatcatctccaggacg

SEQ ID NO: 692 - RNA, 21 nt, synthetic construct
  source 1..21: mol_type = other RNA
tagtgttgatcatctccagga

SEQ ID NO: 693 - RNA, 21 nt, synthetic construct
  source 1..21: mol_type = other RNA
tgagtgttgatcatctccagg

SEQ ID NO: 694 - RNA, 21 nt, synthetic construct
  source 1..21: mol_type = other RNA
tagagtgttgatcatctccag

SEQ ID NO: 695 - RNA, 21 nt, synthetic construct
  source 1..21: mol_type = other RNA
tcagagtgttgatcatctcca

SEQ ID NO: 696 - RNA, 21 nt, synthetic construct
  source 1..21: mol_type = other RNA
tatgatgaagggcagagtgtt

SEQ ID NO: 697 - RNA, 21 nt, synthetic construct
  source 1..21: mol_type = other RNA
tgatgatgaagggcagagtgt

SEQ ID NO: 698 - RNA, 21 nt, synthetic construct
  source 1..21: mol_type = other RNA
ttcgtgatgatgaagggcaga

SEQ ID NO: 699 - RNA, 21 nt, synthetic construct
  source 1..21: mol_type = other RNA
tatcgtgatgatgaagggcag

SEQ ID NO: 700 - RNA, 21 nt, synthetic construct
  source 1..21: mol_type = other RNA
tgatcgtgatgatgaagggca

SEQ ID NO: 701 - RNA, 21 nt, synthetic construct
  source 1..21: mol_type = other RNA
tagaagatcgtgatgatgaag

SEQ ID NO: 702 - RNA, 21 nt, synthetic construct
  source 1..21: mol_type = other RNA
tcaggttccgcagcggcggcc

SEQ ID NO: 703 - RNA, 21 nt, synthetic construct
  source 1..21: mol_type = other RNA
tacaggttccgcagcggcggc

SEQ ID NO: 704 - RNA, 21 nt, synthetic construct
  source 1..21: mol_type = other RNA
taacaggttccgcagcggcgg

SEQ ID NO: 705 - RNA, 21 nt, synthetic construct
  source 1..21: mol_type = other RNA
tgaacaggttccgcagcggcg

SEQ ID NO: 706 - RNA, 21 nt, synthetic construct
  source 1..21: mol_type = other RNA
tcagcgcgtgcttggccagcc

SEQ ID NO: 707 - RNA, 21 nt, synthetic construct
  source 1..21: mol_type = other RNA
tccagcgcgtgcttggccagc

SEQ ID NO: 708 - RNA, 21 nt, synthetic construct
  source 1..21: mol_type = other RNA
ttccagcgcgtgcttggccag

SEQ ID NO: 709 - RNA, 21 nt, synthetic construct
  source 1..21: mol_type = other RNA
taatcatgttttccagcgcgt

SEQ ID NO: 710 - RNA, 21 nt, synthetic construct
  source 1..21: mol_type = other RNA
tgttgaacatggctgactgtg

SEQ ID NO: 711 - RNA, 21 nt, synthetic construct
  source 1..21: mol_type = other RNA
tggttgaacatggctgactgt

SEQ ID NO: 712 - RNA, 21 nt, synthetic construct
  source 1..21: mol_type = other RNA
ttggttgaacatggctgactg

SEQ ID NO: 713 - RNA, 21 nt, synthetic construct
  source 1..21: mol_type = other RNA
tctggttgaacatggctgact

SEQ ID NO: 714 - RNA, 21 nt, synthetic construct
  source 1..21: mol_type = other RNA
tacctggttgaacatggctga

SEQ ID NO: 715 - RNA, 21 nt, synthetic construct
  source 1..21: mol_type = other RNA
tgacctggttgaacatggctg

SEQ ID NO: 716 - RNA, 21 nt, synthetic construct
  source 1..21: mol_type = other RNA
tggacctggttgaacatggct

SEQ ID NO: 717 - RNA, 21 nt, synthetic construct
  source 1..21: mol_type = other RNA
taggacctggttgaacatggc

SEQ ID NO: 718 - RNA, 21 nt, synthetic construct
  source 1..21: mol_type = other RNA
taggatgaggacctggttgaa

SEQ ID NO: 719 - RNA, 21 nt, synthetic construct
  source 1..21: mol_type = other RNA
tcagggtgcagaagaggatga

SEQ ID NO: 720 - RNA, 21 nt, synthetic construct
  source 1..21: mol_type = other RNA
tcagcagggtgcagaagagga

SEQ ID NO: 721 - RNA, 21 nt, synthetic construct
  source 1..21: mol_type = other RNA
tagaaggaggtcaggagggac

SEQ ID NO: 722 - RNA, 21 nt, synthetic construct
  source 1..21: mol_type = other RNA
tgtagaaggaggtcaggaggg

SEQ ID NO: 723 - RNA, 21 nt, synthetic construct
  source 1..21: mol_type = other RNA
taagtagaaggaggtcaggag

SEQ ID NO: 724 - RNA, 21 nt, synthetic construct
  source 1..21: mol_type = other RNA
tcagaagtagaaggaggtcag

SEQ ID NO: 725 - RNA, 21 nt, synthetic construct
  source 1..21: mol_type = other RNA
tgacgatgcagaagtagaagg

SEQ ID NO: 726 - RNA, 21 nt, synthetic construct
  source 1..21: mol_type = other RNA
ttgacgatgcagaagtagaag

SEQ ID NO: 727 - RNA, 21 nt, synthetic construct
  source 1..21: mol_type = other RNA
tgatcatgatgaccaccagca

SEQ ID NO: 728 - RNA, 21 nt, synthetic construct
  source 1..21: mol_type = other RNA
tggaagtccatgagaaggtcg

SEQ ID NO: 729 - RNA, 21 nt, synthetic construct
  source 1..21: mol_type = other RNA
taggaagtccatgagaaggtc

SEQ ID NO: 730 - RNA, 21 nt, synthetic construct
  source 1..21: mol_type = other RNA
tgttcaggaagtccatgagaa

SEQ ID NO: 731 - RNA, 21 nt, synthetic construct
  source 1..21: mol_type = other RNA
tcgttcaggaagtccatgaga

SEQ ID NO: 732 - RNA, 21 nt, synthetic construct
  source 1..21: mol_type = other RNA
ttcgttcaggaagtccatgag

SEQ ID NO: 733 - RNA, 21 nt, synthetic construct
  source 1..21: mol_type = other RNA
tgtaatagtcctggagccggg

SEQ ID NO: 734 - RNA, 21 nt, synthetic construct
  source 1..21: mol_type = other RNA
tatgaccacgtaatagtcctg

SEQ ID NO: 735 - RNA, 21 nt, synthetic construct
  source 1..21: mol_type = other RNA
tttcaggtttgaggatctgga

SEQ ID NO: 736 - RNA, 21 nt, synthetic construct
  source 1..21: mol_type = other RNA
taacttgttttcaggtttgag

SEQ ID NO: 737 - RNA, 21 nt, synthetic construct
  source 1..21: mol_type = other RNA
taaacttgttttcaggtttga

SEQ ID NO: 738 - RNA, 21 nt, synthetic construct
  source 1..21: mol_type = other RNA
tacaccacgtggtcagcaaac

SEQ ID NO: 739 - RNA, 21 nt, synthetic construct
  source 1..21: mol_type = other RNA
tcagatgcagttcagcgccag

SEQ ID NO: 740 - RNA, 21 nt, synthetic construct
  source 1..21: mol_type = other RNA
tgaacttgctgtcacccatgc

SEQ ID NO: 741 - RNA, 21 nt, synthetic construct
  source 1..21: mol_type = other RNA
tagaacttgctgtcacccatg

SEQ ID NO: 742 - RNA, 21 nt, synthetic construct
  source 1..21: mol_type = other RNA
tagcccgatgaggcacacgcc

SEQ ID NO: 743 - RNA, 21 nt, synthetic construct
  source 1..21: mol_type = other RNA
tcttcagcccgatgaggcaca

SEQ ID NO: 744 - RNA, 21 nt, synthetic construct
  source 1..21: mol_type = other RNA
tccgcttcagcccgatgaggc

SEQ ID NO: 745 - RNA, 21 nt, synthetic construct
  source 1..21: mol_type = other RNA
tgatgtagaagcaggtgtcag

SEQ ID NO: 746 - RNA, 21 nt, synthetic construct
  source 1..21: mol_type = other RNA
ttggtgatgttgatgtagaag

SEQ ID NO: 747 - RNA, 21 nt, synthetic construct
  source 1..21: mol_type = other RNA
tcagcccttgtccagccgcag

SEQ ID NO: 748 - RNA, 21 nt, synthetic construct
  source 1..21: mol_type = other RNA
tttgttcttgaacccgtaggc

SEQ ID NO: 749 - RNA, 21 nt, synthetic construct
  source 1..21: mol_type = other RNA
tcttgttcttgaacccgtagg

SEQ ID NO: 750 - RNA, 21 nt, synthetic construct
  source 1..21: mol_type = other RNA
tgatcagcttgttcttgaacc

SEQ ID NO: 751 - RNA, 21 nt, synthetic construct
  source 1..21: mol_type = other RNA
ttgatcagcttgttcttgaac

SEQ ID NO: 752 - RNA, 21 nt, synthetic construct
  source 1..21: mol_type = other RNA
tatgatcagcttgttcttgaa

SEQ ID NO: 753 - RNA, 21 nt, synthetic construct
  source 1..21: mol_type = other RNA
tggatctgtagtaggcccgca

SEQ ID NO: 754 - RNA, 21 nt, synthetic construct
  source 1..21: mol_type = other RNA
tgggatctgtagtaggcccgc

SEQ ID NO: 755 - RNA, 21 nt, synthetic construct
  source 1..21: mol_type = other RNA
tgaagtggtggtcgggcttgt

SEQ ID NO: 756 - RNA, 21 nt, synthetic construct
  source 1..21: mol_type = other RNA
tggaagtggtggtcgggcttg

SEQ ID NO: 757 - RNA, 21 nt, synthetic construct
  source 1..21: mol_type = other RNA
taggaagtggtggtcgggctt

SEQ ID NO: 758 - RNA, 21 nt, synthetic construct
  source 1..21: mol_type = other RNA
tcaggaagtggtggtcgggct

SEQ ID NO: 759 - RNA, 21 nt, synthetic construct
  source 1..21: mol_type = other RNA
tttccaggaagtggtggtcgg

SEQ ID NO: 760 - RNA, 21 nt, synthetic construct
  source 1..21: mol_type = other RNA
tcttccaggaagtggtggtcg

SEQ ID NO: 761 - RNA, 21 nt, synthetic construct
  source 1..21: mol_type = other RNA
tgcttccaggaagtggtggtc

SEQ ID NO: 762 - RNA, 21 nt, synthetic construct
  source 1..21: mol_type = other RNA
tctctccttgtccaccaccac

SEQ ID NO: 763 - RNA, 21 nt, synthetic construct
  source 1..21: mol_type = other RNA
ttagtttggaaagagccagag

SEQ ID NO: 764 - RNA, 21 nt, synthetic construct
  source 1..21: mol_type = other RNA
tctagtttggaaagagccaga

SEQ ID NO: 765 - RNA, 21 nt, synthetic construct
  source 1..21: mol_type = other RNA
ttctagtttggaaagagccag

SEQ ID NO: 766 - RNA, 21 nt, synthetic construct
  source 1..21: mol_type = other RNA
tttctagtttggaaagagcca

SEQ ID NO: 767 - RNA, 21 nt, synthetic construct
  source 1..21: mol_type = other RNA
ttttctagtttggaaagagcc

SEQ ID NO: 768 - RNA, 21 nt, synthetic construct
  source 1..21: mol_type = other RNA
tctcttttctagtttggaaag

SEQ ID NO: 769 - RNA, 21 nt, synthetic construct
  source 1..21: mol_type = other RNA
tccctcttttctagtttggaa

SEQ ID NO: 770 - RNA, 21 nt, synthetic construct
  source 1..21: mol_type = other RNA
ttccctcttttctagtttgga

SEQ ID NO: 771 - RNA, 21 nt, synthetic construct
  source 1..21: mol_type = other RNA
tggcggaacatgaaggccagg

SEQ ID NO: 772 - RNA, 21 nt, synthetic construct
  source 1..21: mol_type = other RNA
tcggcaggcggaacatgaagg

SEQ ID NO: 773 - RNA, 21 nt, synthetic construct
  source 1..21: mol_type = other RNA
tacggcaggcggaacatgaag

SEQ ID NO: 774 - RNA, 21 nt, synthetic construct
  source 1..21: mol_type = other RNA
taacggcaggcggaacatgaa

SEQ ID NO: 775 - RNA, 21 nt, synthetic construct
  source 1..21: mol_type = other RNA
tgaacggcaggcggaacatga

SEQ ID NO: 776 - RNA, 21 nt, synthetic construct
  source 1..21: mol_type = other RNA
tctgatgctgaagacgcggcc

SEQ ID NO: 777 - RNA, 21 nt, synthetic construct
  source 1..21: mol_type = other RNA
tgactggtagagcagtgtgtc

SEQ ID NO: 778 - RNA, 21 nt, synthetic construct
  source 1..21: mol_type = other RNA
tggactggtagagcagtgtgt

SEQ ID NO: 779 - RNA, 21 nt, synthetic construct
  source 1..21: mol_type = other RNA
ttccttcacgaaggactggta

SEQ ID NO: 780 - RNA, 21 nt, synthetic construct
  source 1..21: mol_type = other RNA
tgtccttcacgaaggactggt

SEQ ID NO: 781 - RNA, 21 nt, synthetic construct
  source 1..21: mol_type = other RNA
ttagtccttcacgaaggactg

SEQ ID NO: 782 - RNA, 21 nt, synthetic construct
  source 1..21: mol_type = other RNA
tgtagtccttcacgaaggact

SEQ ID NO: 783 - RNA, 21 nt, synthetic construct
  source 1..21: mol_type = other RNA
ttgtagtccttcacgaaggac

SEQ ID NO: 784 - RNA, 21 nt, synthetic construct
  source 1..21: mol_type = other RNA
tgggtgatggtgatcatgtag

SEQ ID NO: 785 - RNA, 21 nt, synthetic construct
  source 1..21: mol_type = other RNA
tcgggtgatggtgatcatgta

SEQ ID NO: 786 - RNA, 21 nt, synthetic construct
  source 1..21: mol_type = other RNA
tccgggtgatggtgatcatgt

SEQ ID NO: 787 - RNA, 21 nt, synthetic construct
  source 1..21: mol_type = other RNA
tgccgggtgatggtgatcatg

SEQ ID NO: 788 - RNA, 21 nt, synthetic construct
  source 1..21: mol_type = other RNA
tcggtgattttcatggcacag

SEQ ID NO: 789 - RNA, 21 nt, synthetic construct
  source 1..21: mol_type = other RNA
tttcaccgagatctgggactg

SEQ ID NO: 790 - RNA, 21 nt, synthetic construct
  source 1..21: mol_type = other RNA
tgagagctcctggcgctcaga

SEQ ID NO: 791 - RNA, 21 nt, synthetic construct
  source 1..21: mol_type = other RNA
tgcggttcttcaccagctcgg

SEQ ID NO: 792 - RNA, 21 nt, synthetic construct
  source 1..21: mol_type = other RNA
tggtcgttcatctcgtcgtag

SEQ ID NO: 793 - RNA, 21 nt, synthetic construct
  source 1..21: mol_type = other RNA
tccacgtccttcctgaggacc

SEQ ID NO: 794 - RNA, 21 nt, synthetic construct
  source 1..21: mol_type = other RNA
tgtcctcacacgctcctccac

SEQ ID NO: 795 - RNA, 21 nt, synthetic construct
  source 1..21: mol_type = other RNA
tccagggtggagtcccaggag

SEQ ID NO: 796 - RNA, 21 nt, synthetic construct
  source 1..21: mol_type = other RNA
ttccagggtggagtcccagga

SEQ ID NO: 797 - RNA, 21 nt, synthetic construct
  source 1..21: mol_type = other RNA
tttccagggtggagtcccagg

SEQ ID NO: 798 - RNA, 21 nt, synthetic construct
  source 1..21: mol_type = other RNA
ttttccagggtggagtcccag

SEQ ID NO: 799 - RNA, 21 nt, synthetic construct
  source 1..21: mol_type = other RNA
tactctcctcactagcctgca

SEQ ID NO: 800 - RNA, 21 nt, synthetic construct
  source 1..21: mol_type = other RNA
taaactctcctcactagcctg

SEQ ID NO: 801 - RNA, 21 nt, synthetic construct
  source 1..21: mol_type = other RNA
tggactgcatcgacgtgtaaa

SEQ ID NO: 802 - RNA, 21 nt, synthetic construct
  source 1..21: mol_type = other RNA
tacatctgtgtaaagagaagt

SEQ ID NO: 803 - RNA, 21 nt, synthetic construct
  source 1..21: mol_type = other RNA
tgtacatctgtgtaaagagaa

SEQ ID NO: 804 - RNA, 21 nt, synthetic construct
  source 1..21: mol_type = other RNA
tgccctggtcacgagttgcgg

SEQ ID NO: 805 - RNA, 21 nt, synthetic construct
  source 1..21: mol_type = other RNA
tcagaaaggtcctcggcgaca

SEQ ID NO: 806 - RNA, 21 nt, synthetic construct
  source 1..21: mol_type = other RNA
ttggcgtcttctgcttccttc

SEQ ID NO: 807 - RNA, 21 nt, synthetic construct
  source 1..21: mol_type = other RNA
tgaaatggcgtcttctgcttc

SEQ ID NO: 808 - RNA, 21 nt, synthetic construct
  source 1..21: mol_type = other RNA
tggaaatggcgtcttctgctt

SEQ ID NO: 809 - RNA, 21 nt, synthetic construct
  source 1..21: mol_type = other RNA
tagaggaaatggcgtcttctg

SEQ ID NO: 810 - RNA, 21 nt, synthetic construct
  source 1..21: mol_type = other RNA
ttagaggaaatggcgtcttct

SEQ ID NO: 811 - RNA, 21 nt, synthetic construct
  source 1..21: mol_type = other RNA
taagtagaggaaatggcgtct

SEQ ID NO: 812 - RNA, 21 nt, synthetic construct
  source 1..21: mol_type = other RNA
ttgaagtagaggaaatggcgt

SEQ ID NO: 813 - RNA, 21 nt, synthetic construct
  source 1..21: mol_type = other RNA
tgtgtgaagtagaggaaatgg

SEQ ID NO: 814 - RNA, 21 nt, synthetic construct
  source 1..21: mol_type = other RNA
ttcagtgtgaagtagaggaaa

SEQ ID NO: 815 - RNA, 21 nt, synthetic construct
  source 1..21: mol_type = other RNA
tttcagtgtgaagtagaggaa

SEQ ID NO: 816 - RNA, 21 nt, synthetic construct
  source 1..21: mol_type = other RNA
tgaaatgcaccatcttccgcc

SEQ ID NO: 817 - RNA, 21 nt, synthetic construct
  source 1..21: mol_type = other RNA
tggaaatgcaccatcttccgc

SEQ ID NO: 818 - RNA, 21 nt, synthetic construct
  source 1..21: mol_type = other RNA
tggtccatggaaatgcaccat

SEQ ID NO: 819 - RNA, 21 nt, synthetic construct
  source 1..21: mol_type = other RNA
ttaagtgtaaaatggtccatg

SEQ ID NO: 820 - RNA, 21 nt, synthetic construct
  source 1..21: mol_type = other RNA
tgtaagtgtaaaatggtccat

SEQ ID NO: 821 - RNA, 21 nt, synthetic construct
  source 1..21: mol_type = other RNA
tggtaagtgtaaaatggtcca

SEQ ID NO: 822 - RNA, 21 nt, synthetic construct
  source 1..21: mol_type = other RNA
taggtaagtgtaaaatggtcc

SEQ ID NO: 823 - RNA, 21 nt, synthetic construct
  source 1..21: mol_type = other RNA
taaaggtaagtgtaaaatggt

SEQ ID NO: 824 - RNA, 21 nt, synthetic construct
  source 1..21: mol_type = other RNA
taaaaggtaagtgtaaaatgg

SEQ ID NO: 825 - RNA, 21 nt, synthetic construct
  source 1..21: mol_type = other RNA
ttaaaaggtaagtgtaaaatg

SEQ ID NO: 826 - RNA, 21 nt, synthetic construct
  source 1..21: mol_type = other RNA
tttaaaaggtaagtgtaaaat

SEQ ID NO: 827 - RNA, 21 nt, synthetic construct
  source 1..21: mol_type = other RNA
ttttaaaaggtaagtgtaaaa

SEQ ID NO: 828 - RNA, 21 nt, synthetic construct
  source 1..21: mol_type = other RNA
ttgctttaaaaggtaagtgta

SEQ ID NO: 829 - RNA, 21 nt, synthetic construct
  source 1..21: mol_type = other RNA
tttgctttaaaaggtaagtgt

SEQ ID NO: 830 - RNA, 21 nt, synthetic construct
  source 1..21: mol_type = other RNA
ttttgctttaaaaggtaagtg

SEQ ID NO: 831 - RNA, 21 nt, synthetic construct
  source 1..21: mol_type = other RNA
tctttgctttaaaaggtaagt

SEQ ID NO: 832 - RNA, 21 nt, synthetic construct
  source 1..21: mol_type = other RNA
tgctttgctttaaaaggtaag

SEQ ID NO: 833 - RNA, 21 nt, synthetic construct
  source 1..21: mol_type = other RNA
taggctttgctttaaaaggta

SEQ ID NO: 834 - RNA, 21 nt, synthetic construct
  source 1..21: mol_type = other RNA
tatgaggctttgctttaaaag

SEQ ID NO: 835 - RNA, 21 nt, synthetic construct
  source 1..21: mol_type = other RNA
taaaatgaggctttgctttaa

SEQ ID NO: 836 - RNA, 21 nt, synthetic construct
  source 1..21: mol_type = other RNA
tgggatctcgccttgctgaga

SEQ ID NO: 837 - RNA, 21 nt, synthetic construct
  source 1..21: mol_type = other RNA
tagcaaaggacgcagggtggc

SEQ ID NO: 838 - RNA, 21 nt, synthetic construct
  source 1..21: mol_type = other RNA
taaggaaccagcagcaaagga

SEQ ID NO: 839 - RNA, 21 nt, synthetic construct
  source 1..21: mol_type = other RNA
tcagtaaggaaccagcagcaa

SEQ ID NO: 840 - RNA, 21 nt, synthetic construct
  source 1..21: mol_type = other RNA
tgctgaccgtacaaaccagta

SEQ ID NO: 841 - RNA, 21 nt, synthetic construct
  source 1..21: mol_type = other RNA
tatagaagtttccagcgctga

SEQ ID NO: 842 - RNA, 21 nt, synthetic construct
  source 1..21: mol_type = other RNA
taatagaagtttccagcgctg

SEQ ID NO: 843 - RNA, 21 nt, synthetic construct
  source 1..21: mol_type = other RNA
ttttaatagaagtttccagcg

SEQ ID NO: 844 - RNA, 21 nt, synthetic construct
  source 1..21: mol_type = other RNA
tagaatgcatccatttaatag

SEQ ID NO: 845 - RNA, 16 nt, synthetic construct
  source 1..16: mol_type = other RNA
ctcctggacaccgcca

SEQ ID NO: 846 - RNA, 16 nt, synthetic construct
  source 1..16: mol_type = other RNA
aggtgctgcccttgca

SEQ ID NO: 847 - RNA, 16 nt, synthetic construct
  source 1..16: mol_type = other RNA
tccttccagaacgaca

SEQ ID NO: 848 - RNA, 16 nt, synthetic construct
  source 1..16: mol_type = other RNA
gtccaggtggagttca

SEQ ID NO: 849 - RNA, 16 nt, synthetic construct
  source 1..16: mol_type = other RNA
tccaggtggagttcta

SEQ ID NO: 850 - RNA, 16 nt, synthetic construct
  source 1..16: mol_type = other RNA
ccaggtggagttctaa

SEQ ID NO: 851 - RNA, 16 nt, synthetic construct
  source 1..16: mol_type = other RNA
tcaaggagcggctcaa

SEQ ID NO: 852 - RNA, 16 nt, synthetic construct
  source 1..16: mol_type = other RNA
ctgaggatccggctga

SEQ ID NO: 853 - RNA, 16 nt, synthetic construct
  source 1..16: mol_type = other RNA
tgaggatccggctgta

SEQ ID NO: 854 - RNA, 16 nt, synthetic construct
  source 1..16: mol_type = other RNA
gaggatccggctgtta

SEQ ID NO: 855 - RNA, 16 nt, synthetic construct
  source 1..16: mol_type = other RNA
aggatccggctgttca

SEQ ID NO: 856 - RNA, 16 nt, synthetic construct
  source 1..16: mol_type = other RNA
ttcaacttctccctga

SEQ ID NO: 857 - RNA, 16 nt, synthetic construct
  source 1..16: mol_type = other RNA
caacttctccctgaaa

SEQ ID NO: 858 - RNA, 16 nt, synthetic construct
  source 1..16: mol_type = other RNA
aacttctccctgaaga

SEQ ID NO: 859 - RNA, 16 nt, synthetic construct
  source 1..16: mol_type = other RNA
ccaaagcagaactaca

SEQ ID NO: 860 - RNA, 16 nt, synthetic construct
  source 1..16: mol_type = other RNA
aaagcagaactactca

SEQ ID NO: 861 - RNA, 16 nt, synthetic construct
  source 1..16: mol_type = other RNA
ccttcaatgactcgta

SEQ ID NO: 862 - RNA, 16 nt, synthetic construct
  source 1..16: mol_type = other RNA
caatgactcgtcctca

SEQ ID NO: 863 - RNA, 16 nt, synthetic construct
  source 1..16: mol_type = other RNA
tccgagatcaactgga

SEQ ID NO: 864 - RNA, 16 nt, synthetic construct
  source 1..16: mol_type = other RNA
ccgagatcaactggga

SEQ ID NO: 865 - RNA, 16 nt, synthetic construct
  source 1..16: mol_type = other RNA
agatcaactgggctca

SEQ ID NO: 866 - RNA, 16 nt, synthetic construct
  source 1..16: mol_type = other RNA
ggagagaaagatgaca

SEQ ID NO: 867 - RNA, 16 nt, synthetic construct
  source 1..16: mol_type = other RNA
agaaagatgacactga

SEQ ID NO: 868 - RNA, 16 nt, synthetic construct
  source 1..16: mol_type = other RNA
aaagatgacactgtga

SEQ ID NO: 869 - RNA, 16 nt, synthetic construct
  source 1..16: mol_type = other RNA
ccaggtcatcgtggca

SEQ ID NO: 870 - RNA, 16 nt, synthetic construct
  source 1..16: mol_type = other RNA
gccataataagcttca

SEQ ID NO: 871 - RNA, 16 nt, synthetic construct
  source 1..16: mol_type = other RNA
ccataataagcttcca

SEQ ID NO: 872 - RNA, 16 nt, synthetic construct
  source 1..16: mol_type = other RNA
ataataagcttcctga

SEQ ID NO: 873 - RNA, 16 nt, synthetic construct
  source 1..16: mol_type = other RNA
cttctcatctacctca

SEQ ID NO: 874 - RNA, 16 nt, synthetic construct
  source 1..16: mol_type = other RNA
caacatctgggagcaa

SEQ ID NO: 875 - RNA, 16 nt, synthetic construct
  source 1..16: mol_type = other RNA
aacatctgggagcaga

SEQ ID NO: 876 - RNA, 16 nt, synthetic construct
  source 1..16: mol_type = other RNA
gcgtgtccttcgtcca

SEQ ID NO: 877 - RNA, 16 nt, synthetic construct
  source 1..16: mol_type = other RNA
gtgtccttcgtcctga

SEQ ID NO: 878 - RNA, 16 nt, synthetic construct
  source 1..16: mol_type = other RNA
tcctggagatgatcaa

SEQ ID NO: 879 - RNA, 16 nt, synthetic construct
  source 1..16: mol_type = other RNA
cctggagatgatcaaa

SEQ ID NO: 880 - RNA, 16 nt, synthetic construct
  source 1..16: mol_type = other RNA
ctggagatgatcaaca

SEQ ID NO: 881 - RNA, 16 nt, synthetic construct
  source 1..16: mol_type = other RNA
tggagatgatcaacaa

SEQ ID NO: 882 - RNA, 16 nt, synthetic construct
  source 1..16: mol_type = other RNA
gagatgatcaacacta

SEQ ID NO: 883 - RNA, 16 nt, synthetic construct
  source 1..16: mol_type = other RNA
agatgatcaacactca

SEQ ID NO: 884 - RNA, 16 nt, synthetic construct
  source 1..16: mol_type = other RNA
gatgatcaacactcta

SEQ ID NO: 885 - RNA, 16 nt, synthetic construct
  source 1..16: mol_type = other RNA
atgatcaacactctga

SEQ ID NO: 886 - RNA, 16 nt, synthetic construct
  source 1..16: mol_type = other RNA
tctgcccttcatcata

SEQ ID NO: 887 - RNA, 16 nt, synthetic construct
  source 1..16: mol_type = other RNA
ctgcccttcatcatca

SEQ ID NO: 888 - RNA, 16 nt, synthetic construct
  source 1..16: mol_type = other RNA
ccttcatcatcacgaa

SEQ ID NO: 889 - RNA, 16 nt, synthetic construct
  source 1..16: mol_type = other RNA
cttcatcatcacgata

SEQ ID NO: 890 - RNA, 16 nt, synthetic construct
  source 1..16: mol_type = other RNA
ttcatcatcacgatca

SEQ ID NO: 891 - RNA, 16 nt, synthetic construct
  source 1..16: mol_type = other RNA
tcatcacgatcttcta

SEQ ID NO: 892 - RNA, 16 nt, synthetic construct
  source 1..16: mol_type = other RNA
ccgctgcggaacctga

SEQ ID NO: 893 - RNA, 16 nt, synthetic construct
  source 1..16: mol_type = other RNA
cgctgcggaacctgta

SEQ ID NO: 894 - RNA, 16 nt, synthetic construct
  source 1..16: mol_type = other RNA
gctgcggaacctgtta

SEQ ID NO: 895 - RNA, 16 nt, synthetic construct
  source 1..16: mol_type = other RNA
ctgcggaacctgttca

SEQ ID NO: 896 - RNA, 16 nt, synthetic construct
  source 1..16: mol_type = other RNA
gccaagcacgcgctga

SEQ ID NO: 897 - RNA, 16 nt, synthetic construct
  source 1..16: mol_type = other RNA
ccaagcacgcgctgga

SEQ ID NO: 898 - RNA, 16 nt, synthetic construct
  source 1..16: mol_type = other RNA
caagcacgcgctggaa

SEQ ID NO: 899 - RNA, 16 nt, synthetic construct
  source 1..16: mol_type = other RNA
ctggaaaacatgatta

SEQ ID NO: 900 - RNA, 16 nt, synthetic construct
  source 1..16: mol_type = other RNA
tcagccatgttcaaca

SEQ ID NO: 901 - RNA, 16 nt, synthetic construct
  source 1..16: mol_type = other RNA
cagccatgttcaacca

SEQ ID NO: 902 - RNA, 16 nt, synthetic construct
  source 1..16: mol_type = other RNA
agccatgttcaaccaa

SEQ ID NO: 903 - RNA, 16 nt, synthetic construct
  source 1..16: mol_type = other RNA
gccatgttcaaccaga

SEQ ID NO: 904 - RNA, 16 nt, synthetic construct
  source 1..16: mol_type = other RNA
catgttcaaccaggta

SEQ ID NO: 905 - RNA, 16 nt, synthetic construct
  source 1..16: mol_type = other RNA
atgttcaaccaggtca

SEQ ID NO: 906 - RNA, 16 nt, synthetic construct
  source 1..16: mol_type = other RNA
tgttcaaccaggtcca

SEQ ID NO: 907 - RNA, 16 nt, synthetic construct
  source 1..16: mol_type = other RNA
gttcaaccaggtccta

SEQ ID NO: 908 - RNA, 16 nt, synthetic construct
  source 1..16: mol_type = other RNA
ccaggtcctcatccta

SEQ ID NO: 909 - RNA, 16 nt, synthetic construct
  source 1..16: mol_type = other RNA
ctcttctgcaccctga

SEQ ID NO: 910 - RNA, 16 nt, synthetic construct
  source 1..16: mol_type = other RNA
ttctgcaccctgctga

SEQ ID NO: 911 - RNA, 16 nt, synthetic construct
  source 1..16: mol_type = other RNA
tcctgacctccttcta

SEQ ID NO: 912 - RNA, 16 nt, synthetic construct
  source 1..16: mol_type = other RNA
ctgacctccttctaca

SEQ ID NO: 913 - RNA, 16 nt, synthetic construct
  source 1..16: mol_type = other RNA
gacctccttctactta

SEQ ID NO: 914 - RNA, 16 nt, synthetic construct
  source 1..16: mol_type = other RNA
ctccttctacttctga

SEQ ID NO: 915 - RNA, 16 nt, synthetic construct
  source 1..16: mol_type = other RNA
tacttctgcatcgtca

SEQ ID NO: 916 - RNA, 16 nt, synthetic construct
  source 1..16: mol_type = other RNA
acttctgcatcgtcaa

SEQ ID NO: 917 - RNA, 16 nt, synthetic construct
  source 1..16: mol_type = other RNA
gtggtcatcatgatca

SEQ ID NO: 918 - RNA, 16 nt, synthetic construct
  source 1..16: mol_type = other RNA
ttctcatggacttcca

SEQ ID NO: 919 - RNA, 16 nt, synthetic construct
  source 1..16: mol_type = other RNA
tctcatggacttccta

SEQ ID NO: 920 - RNA, 16 nt, synthetic construct
  source 1..16: mol_type = other RNA
atggacttcctgaaca

SEQ ID NO: 921 - RNA, 16 nt, synthetic construct
  source 1..16: mol_type = other RNA
tggacttcctgaacga

SEQ ID NO: 922 - RNA, 16 nt, synthetic construct
  source 1..16: mol_type = other RNA
ggacttcctgaacgaa

SEQ ID NO: 923 - RNA, 16 nt, synthetic construct
  source 1..16: mol_type = other RNA
ctccaggactattaca

SEQ ID NO: 924 - RNA, 16 nt, synthetic construct
  source 1..16: mol_type = other RNA
ctattacgtggtcata

SEQ ID NO: 925 - RNA, 16 nt, synthetic construct
  source 1..16: mol_type = other RNA
atcctcaaacctgaaa

SEQ ID NO: 926 - RNA, 16 nt, synthetic construct
  source 1..16: mol_type = other RNA
acctgaaaacaagtta

SEQ ID NO: 927 - RNA, 16 nt, synthetic construct
  source 1..16: mol_type = other RNA
cctgaaaacaagttta

SEQ ID NO: 928 - RNA, 16 nt, synthetic construct
  source 1..16: mol_type = other RNA
ctgaccacgtggtgta

SEQ ID NO: 929 - RNA, 16 nt, synthetic construct
  source 1..16: mol_type = other RNA
gctgaactgcatctga

SEQ ID NO: 930 - RNA, 16 nt, synthetic construct
  source 1..16: mol_type = other RNA
ggtgacagcaagttca

SEQ ID NO: 931 - RNA, 16 nt, synthetic construct
  source 1..16: mol_type = other RNA
gtgacagcaagttcta

SEQ ID NO: 932 - RNA, 16 nt, synthetic construct
  source 1..16: mol_type = other RNA
gtgcctcatcgggcta

SEQ ID NO: 933 - RNA, 16 nt, synthetic construct
  source 1..16: mol_type = other RNA
ctcatcgggctgaaga

SEQ ID NO: 934 - RNA, 16 nt, synthetic construct
  source 1..16: mol_type = other RNA
atcgggctgaagcgga

SEQ ID NO: 935 - RNA, 16 nt, synthetic construct
  source 1..16: mol_type = other RNA
acctgcttctacatca

SEQ ID NO: 936 - RNA, 16 nt, synthetic construct
  source 1..16: mol_type = other RNA
acatcaacatcaccaa

SEQ ID NO: 937 - RNA, 16 nt, synthetic construct
  source 1..16: mol_type = other RNA
gctggacaagggctga

SEQ ID NO: 938 - RNA, 16 nt, synthetic construct
  source 1..16: mol_type = other RNA
cgggttcaagaacaaa

SEQ ID NO: 939 - RNA, 16 nt, synthetic construct
  source 1..16: mol_type = other RNA
gggttcaagaacaaga

SEQ ID NO: 940 - RNA, 16 nt, synthetic construct
  source 1..16: mol_type = other RNA
aagaacaagctgatca

SEQ ID NO: 941 - RNA, 16 nt, synthetic construct
  source 1..16: mol_type = other RNA
agaacaagctgatcaa

SEQ ID NO: 942 - RNA, 16 nt, synthetic construct
  source 1..16: mol_type = other RNA
gaacaagctgatcata

SEQ ID NO: 943 - RNA, 16 nt, synthetic construct
  source 1..16: mol_type = other RNA
gcctactacagatcca

SEQ ID NO: 944 - RNA, 16 nt, synthetic construct
  source 1..16: mol_type = other RNA
cctactacagatccca

SEQ ID NO: 945 - RNA, 16 nt, synthetic construct
  source 1..16: mol_type = other RNA
cccgaccaccacttca

SEQ ID NO: 946 - RNA, 16 nt, synthetic construct
  source 1..16: mol_type = other RNA
ccgaccaccacttcca

SEQ ID NO: 947 - RNA, 16 nt, synthetic construct
  source 1..16: mol_type = other RNA
cgaccaccacttccta

SEQ ID NO: 948 - RNA, 16 nt, synthetic construct
  source 1..16: mol_type = other RNA
gaccaccacttcctga

SEQ ID NO: 949 - RNA, 16 nt, synthetic construct
  source 1..16: mol_type = other RNA
caccacttcctggaaa

SEQ ID NO: 950 - RNA, 16 nt, synthetic construct
  source 1..16: mol_type = other RNA
accacttcctggaaga

SEQ ID NO: 951 - RNA, 16 nt, synthetic construct
  source 1..16: mol_type = other RNA
ccacttcctggaagca

SEQ ID NO: 952 - RNA, 16 nt, synthetic construct
  source 1..16: mol_type = other RNA
ggtggacaaggagaga

SEQ ID NO: 953 - RNA, 16 nt, synthetic construct
  source 1..16: mol_type = other RNA
gctctttccaaactaa

SEQ ID NO: 954 - RNA, 16 nt, synthetic construct
  source 1..16: mol_type = other RNA
ctctttccaaactaga

SEQ ID NO: 955 - RNA, 16 nt, synthetic construct
  source 1..16: mol_type = other RNA
tctttccaaactagaa

SEQ ID NO: 956 - RNA, 16 nt, synthetic construct
  source 1..16: mol_type = other RNA
ctttccaaactagaaa

SEQ ID NO: 957 - RNA, 16 nt, synthetic construct
  source 1..16: mol_type = other RNA
tttccaaactagaaaa

SEQ ID NO: 958 - RNA, 16 nt, synthetic construct
  source 1..16: mol_type = other RNA
caaactagaaaagaga

SEQ ID NO: 959 - RNA, 16 nt, synthetic construct
  source 1..16: mol_type = other RNA
aactagaaaagaggga

SEQ ID NO: 960 - RNA, 16 nt, synthetic construct
  source 1..16: mol_type = other RNA
actagaaaagagggaa

SEQ ID NO: 961 - RNA, 16 nt, synthetic construct
  source 1..16: mol_type = other RNA
ccttcatgttccgcca

SEQ ID NO: 962 - RNA, 16 nt, synthetic construct
  source 1..16: mol_type = other RNA
atgttccgcctgccga

SEQ ID NO: 963 - RNA, 16 nt, synthetic construct
  source 1..16: mol_type = other RNA
tgttccgcctgccgta

SEQ ID NO: 964 - RNA, 16 nt, synthetic construct
  source 1..16: mol_type = other RNA
gttccgcctgccgtta

SEQ ID NO: 965 - RNA, 16 nt, synthetic construct
  source 1..16: mol_type = other RNA
ttccgcctgccgttca

SEQ ID NO: 966 - RNA, 16 nt, synthetic construct
  source 1..16: mol_type = other RNA
cgtcttcagcatcaga

SEQ ID NO: 967 - RNA, 16 nt, synthetic construct
  source 1..16: mol_type = other RNA
actgctctaccagtca

SEQ ID NO: 968 - RNA, 16 nt, synthetic construct
  source 1..16: mol_type = other RNA
ctgctctaccagtcca

SEQ ID NO: 969 - RNA, 16 nt, synthetic construct
  source 1..16: mol_type = other RNA
gtccttcgtgaaggaa

SEQ ID NO: 970 - RNA, 16 nt, synthetic construct
  source 1..16: mol_type = other RNA
tccttcgtgaaggaca

SEQ ID NO: 971 - RNA, 16 nt, synthetic construct
  source 1..16: mol_type = other RNA
cttcgtgaaggactaa

SEQ ID NO: 972 - RNA, 16 nt, synthetic construct
  source 1..16: mol_type = other RNA
ttcgtgaaggactaca

SEQ ID NO: 973 - RNA, 16 nt, synthetic construct
  source 1..16: mol_type = other RNA
tcgtgaaggactacaa

SEQ ID NO: 974 - RNA, 16 nt, synthetic construct
  source 1..16: mol_type = other RNA
tgatcaccatcaccca

SEQ ID NO: 975 - RNA, 16 nt, synthetic construct
  source 1..16: mol_type = other RNA
gatcaccatcacccga

SEQ ID NO: 976 - RNA, 16 nt, synthetic construct
  source 1..16: mol_type = other RNA
atcaccatcacccgga

SEQ ID NO: 977 - RNA, 16 nt, synthetic construct
  source 1..16: mol_type = other RNA
tcaccatcacccggca

SEQ ID NO: 978 - RNA, 16 nt, synthetic construct
  source 1..16: mol_type = other RNA
ccatgaaaatcaccga

SEQ ID NO: 979 - RNA, 16 nt, synthetic construct
  source 1..16: mol_type = other RNA
ccagatctcggtgaaa

SEQ ID NO: 980 - RNA, 16 nt, synthetic construct
  source 1..16: mol_type = other RNA
gcgccaggagctctca

SEQ ID NO: 981 - RNA, 16 nt, synthetic construct
  source 1..16: mol_type = other RNA
ctggtgaagaaccgca

SEQ ID NO: 982 - RNA, 16 nt, synthetic construct
  source 1..16: mol_type = other RNA
acgagatgaacgacca

SEQ ID NO: 983 - RNA, 16 nt, synthetic construct
  source 1..16: mol_type = other RNA
tcaggaaggacgtgga

SEQ ID NO: 984 - RNA, 16 nt, synthetic construct
  source 1..16: mol_type = other RNA
ggagcgtgtgaggaca

SEQ ID NO: 985 - RNA, 16 nt, synthetic construct
  source 1..16: mol_type = other RNA
gggactccaccctgga

SEQ ID NO: 986 - RNA, 16 nt, synthetic construct
  source 1..16: mol_type = other RNA
ggactccaccctggaa

SEQ ID NO: 987 - RNA, 16 nt, synthetic construct
  source 1..16: mol_type = other RNA
gactccaccctggaaa

SEQ ID NO: 988 - RNA, 16 nt, synthetic construct
  source 1..16: mol_type = other RNA
actccaccctggaaaa

SEQ ID NO: 989 - RNA, 16 nt, synthetic construct
  source 1..16: mol_type = other RNA
gctagtgaggagagta

SEQ ID NO: 990 - RNA, 16 nt, synthetic construct
  source 1..16: mol_type = other RNA
tagtgaggagagttta

SEQ ID NO: 991 - RNA, 16 nt, synthetic construct
  source 1..16: mol_type = other RNA
acgtcgatgcagtcca

SEQ ID NO: 992 - RNA, 16 nt, synthetic construct
  source 1..16: mol_type = other RNA
tctttacacagatgta

SEQ ID NO: 993 - RNA, 16 nt, synthetic construct
  source 1..16: mol_type = other RNA
tttacacagatgtaca

SEQ ID NO: 994 - RNA, 16 nt, synthetic construct
  source 1..16: mol_type = other RNA
actcgtgaccagggca

SEQ ID NO: 995 - RNA, 16 nt, synthetic construct
  source 1..16: mol_type = other RNA
ccgaggacctttctga

SEQ ID NO: 996 - RNA, 16 nt, synthetic construct
  source 1..16: mol_type = other RNA
aagcagaagacgccaa

SEQ ID NO: 997 - RNA, 16 nt, synthetic construct
  source 1..16: mol_type = other RNA
agaagacgccatttca

SEQ ID NO: 998 - RNA, 16 nt, synthetic construct
  source 1..16: mol_type = other RNA
gaagacgccatttcca

SEQ ID NO: 999 - RNA, 16 nt, synthetic construct
  source 1..16: mol_type = other RNA
gacgccatttcctcta

SEQ ID NO: 1000 - RNA, 16 nt, synthetic construct
  source 1..16: mol_type = other RNA
acgccatttcctctaa

SEQ ID NO: 1001 - RNA, 16 nt, synthetic construct
  source 1..16: mol_type = other RNA
ccatttcctctactta

SEQ ID NO: 1002 - RNA, 16 nt, synthetic construct
  source 1..16: mol_type = other RNA
atttcctctacttcaa

SEQ ID NO: 1003 - RNA, 16 nt, synthetic construct
  source 1..16: mol_type = other RNA
tcctctacttcacaca

SEQ ID NO: 1004 - RNA, 16 nt, synthetic construct
  source 1..16: mol_type = other RNA
tctacttcacactgaa

SEQ ID NO: 1005 - RNA, 16 nt, synthetic construct
  source 1..16: mol_type = other RNA
ctacttcacactgaaa

SEQ ID NO: 1006 - RNA, 16 nt, synthetic construct
  source 1..16: mol_type = other RNA
aagatggtgcatttca

SEQ ID NO: 1007 - RNA, 16 nt, synthetic construct
  source 1..16: mol_type = other RNA
agatggtgcatttcca

SEQ ID NO: 1008 - RNA, 16 nt, synthetic construct
  source 1..16: mol_type = other RNA
gcatttccatggacca

SEQ ID NO: 1009 - RNA, 16 nt, synthetic construct
  source 1..16: mol_type = other RNA
accattttacacttaa

SEQ ID NO: 1010 - RNA, 16 nt, synthetic construct
  source 1..16: mol_type = other RNA
ccattttacacttaca

SEQ ID NO: 1011 - RNA, 16 nt, synthetic construct
  source 1..16: mol_type = other RNA
cattttacacttacca

SEQ ID NO: 1012 - RNA, 16 nt, synthetic construct
  source 1..16: mol_type = other RNA
attttacacttaccta

SEQ ID NO: 1013 - RNA, 16 nt, synthetic construct
  source 1..16: mol_type = other RNA
tttacacttaccttta

SEQ ID NO: 1014 - RNA, 16 nt, synthetic construct
  source 1..16: mol_type = other RNA
ttacacttacctttta

SEQ ID NO: 1015 - RNA, 16 nt, synthetic construct
  source 1..16: mol_type = other RNA
tacacttaccttttaa

SEQ ID NO: 1016 - RNA, 16 nt, synthetic construct
  source 1..16: mol_type = other RNA
acacttaccttttaaa

SEQ ID NO: 1017 - RNA, 16 nt, synthetic construct
  source 1..16: mol_type = other RNA
cacttaccttttaaaa

SEQ ID NO: 1018 - RNA, 16 nt, synthetic construct
  source 1..16: mol_type = other RNA
ttaccttttaaagcaa

SEQ ID NO: 1019 - RNA, 16 nt, synthetic construct
  source 1..16: mol_type = other RNA
taccttttaaagcaaa

SEQ ID NO: 1020 - RNA, 16 nt, synthetic construct
  source 1..16: mol_type = other RNA
accttttaaagcaaaa

SEQ ID NO: 1021 - RNA, 16 nt, synthetic construct
  source 1..16: mol_type = other RNA
ccttttaaagcaaaga

SEQ ID NO: 1022 - RNA, 16 nt, synthetic construct
  source 1..16: mol_type = other RNA
cttttaaagcaaagca

SEQ ID NO: 1023 - RNA, 16 nt, synthetic construct
  source 1..16: mol_type = other RNA
tttaaagcaaagccta

SEQ ID NO: 1024 - RNA, 16 nt, synthetic construct
  source 1..16: mol_type = other RNA
aaagcaaagcctcata

SEQ ID NO: 1025 - RNA, 16 nt, synthetic construct
  source 1..16: mol_type = other RNA
gcaaagcctcatttta

SEQ ID NO: 1026 - RNA, 16 nt, synthetic construct
  source 1..16: mol_type = other RNA
gcaaggcgagatccca

SEQ ID NO: 1027 - RNA, 16 nt, synthetic construct
  source 1..16: mol_type = other RNA
cctgcgtcctttgcta

SEQ ID NO: 1028 - RNA, 16 nt, synthetic construct
  source 1..16: mol_type = other RNA
tgctgctggttcctta

SEQ ID NO: 1029 - RNA, 16 nt, synthetic construct
  source 1..16: mol_type = other RNA
gctggttccttactga

SEQ ID NO: 1030 - RNA, 16 nt, synthetic construct
  source 1..16: mol_type = other RNA
gtttgtacggtcagca

SEQ ID NO: 1031 - RNA, 16 nt, synthetic construct
  source 1..16: mol_type = other RNA
gctggaaacttctata

SEQ ID NO: 1032 - RNA, 16 nt, synthetic construct
  source 1..16: mol_type = other RNA
ctggaaacttctatta

SEQ ID NO: 1033 - RNA, 16 nt, synthetic construct
  source 1..16: mol_type = other RNA
gaaacttctattaaaa

SEQ ID NO: 1034 - RNA, 16 nt, synthetic construct
  source 1..16: mol_type = other RNA
aaatggatgcattcta

SEQ ID NO: 1035 - RNA, 21 nt, Homo sapiens
  source 1..21: mol_type = mRNA
gcgcgctcctggacaccgccg

SEQ ID NO: 1036 - RNA, 21 nt, Homo sapiens
  source 1..21: mol_type = mRNA
ctccgaggtgctgcccttgcc

SEQ ID NO: 1037 - RNA, 21 nt, Homo sapiens
  source 1..21: mol_type = mRNA
acccgtccttccagaacgacg

SEQ ID NO: 1038 - RNA, 21 nt, Homo sapiens
  source 1..21: mol_type = mRNA
acagggtccaggtggagttct

SEQ ID NO: 1039 - RNA, 21 nt, Homo sapiens
  source 1..21: mol_type = mRNA
cagggtccaggtggagttcta

SEQ ID NO: 1040 - RNA, 21 nt, Homo sapiens
  source 1..21: mol_type = mRNA
agggtccaggtggagttctac

SEQ ID NO: 1041 - RNA, 21 nt, Homo sapiens
  source 1..21: mol_type = mRNA
caccttcaaggagcggctcaa

SEQ ID NO: 1042 - RNA, 21 nt, Homo sapiens
  source 1..21: mol_type = mRNA
cgagcctgaggatccggctgt

SEQ ID NO: 1043 - RNA, 21 nt, Homo sapiens
  source 1..21: mol_type = mRNA
gagcctgaggatccggctgtt

SEQ ID NO: 1044 - RNA, 21 nt, Homo sapiens
  source 1..21: mol_type = mRNA
agcctgaggatccggctgttc

SEQ ID NO: 1045 - RNA, 21 nt, Homo sapiens
  source 1..21: mol_type = mRNA
gcctgaggatccggctgttca

SEQ ID NO: 1046 - RNA, 21 nt, Homo sapiens
  source 1..21: mol_type = mRNA
ggctgttcaacttctccctga

SEQ ID NO: 1047 - RNA, 21 nt, Homo sapiens
  source 1..21: mol_type = mRNA
ctgttcaacttctccctgaag

SEQ ID NO: 1048 - RNA, 21 nt, Homo sapiens
  source 1..21: mol_type = mRNA
tgttcaacttctccctgaagc

SEQ ID NO: 1049 - RNA, 21 nt, Homo sapiens
  source 1..21: mol_type = mRNA
gctgcccaaagcagaactact

SEQ ID NO: 1050 - RNA, 21 nt, Homo sapiens
  source 1..21: mol_type = mRNA
tgcccaaagcagaactactcc

SEQ ID NO: 1051 - RNA, 21 nt, Homo sapiens
  source 1..21: mol_type = mRNA
ctactccttcaatgactcgtc

SEQ ID NO: 1052 - RNA, 21 nt, Homo sapiens
  source 1..21: mol_type = mRNA
tccttcaatgactcgtcctcc

SEQ ID NO: 1053 - RNA, 21 nt, Homo sapiens
  source 1..21: mol_type = mRNA
cgtcctccgagatcaactggg

SEQ ID NO: 1054 - RNA, 21 nt, Homo sapiens
  source 1..21: mol_type = mRNA
gtcctccgagatcaactgggc

SEQ ID NO: 1055 - RNA, 21 nt, Homo sapiens
  source 1..21: mol_type = mRNA
ctccgagatcaactgggctcc

SEQ ID NO: 1056 - RNA, 21 nt, Homo sapiens
  source 1..21: mol_type = mRNA
tgggtggagagaaagatgaca

SEQ ID NO: 1057 - RNA, 21 nt, Homo sapiens
  source 1..21: mol_type = mRNA
tggagagaaagatgacactgt

SEQ ID NO: 1058 - RNA, 21 nt, Homo sapiens
  source 1..21: mol_type = mRNA
gagagaaagatgacactgtgg

SEQ ID NO: 1059 - RNA, 21 nt, Homo sapiens
  source 1..21: mol_type = mRNA
gcgatccaggtcatcgtggcc

SEQ ID NO: 1060 - RNA, 21 nt, Homo sapiens
  source 1..21: mol_type = mRNA
tcgtggccataataagcttcc

SEQ ID NO: 1061 - RNA, 21 nt, Homo sapiens
  source 1..21: mol_type = mRNA
cgtggccataataagcttcct

SEQ ID NO: 1062 - RNA, 21 nt, Homo sapiens
  source 1..21: mol_type = mRNA
tggccataataagcttcctgg

SEQ ID NO: 1063 - RNA, 21 nt, Homo sapiens
  source 1..21: mol_type = mRNA
cgatgcttctcatctacctca

SEQ ID NO: 1064 - RNA, 21 nt, Homo sapiens
  source 1..21: mol_type = mRNA
aaaggcaacatctgggagcag

SEQ ID NO: 1065 - RNA, 21 nt, Homo sapiens
  source 1..21: mol_type = mRNA
aaggcaacatctgggagcaga

SEQ ID NO: 1066 - RNA, 21 nt, Homo sapiens
  source 1..21: mol_type = mRNA
cttccgcgtgtccttcgtcct

SEQ ID NO: 1067 - RNA, 21 nt, Homo sapiens
  source 1..21: mol_type = mRNA
tccgcgtgtccttcgtcctgg

SEQ ID NO: 1068 - RNA, 21 nt, Homo sapiens
  source 1..21: mol_type = mRNA
cttcgtcctggagatgatcaa

SEQ ID NO: 1069 - RNA, 21 nt, Homo sapiens
  source 1..21: mol_type = mRNA
ttcgtcctggagatgatcaac

SEQ ID NO: 1070 - RNA, 21 nt, Homo sapiens
  source 1..21: mol_type = mRNA
tcgtcctggagatgatcaaca

SEQ ID NO: 1071 - RNA, 21 nt, Homo sapiens
  source 1..21: mol_type = mRNA
cgtcctggagatgatcaacac

SEQ ID NO: 1072 - RNA, 21 nt, Homo sapiens
  source 1..21: mol_type = mRNA
tcctggagatgatcaacactc

SEQ ID NO: 1073 - RNA, 21 nt, Homo sapiens
  source 1..21: mol_type = mRNA
cctggagatgatcaacactct

SEQ ID NO: 1074 - RNA, 21 nt, Homo sapiens
  source 1..21: mol_type = mRNA
ctggagatgatcaacactctg

SEQ ID NO: 1075 - RNA, 21 nt, Homo sapiens
  source 1..21: mol_type = mRNA
tggagatgatcaacactctgc

SEQ ID NO: 1076 - RNA, 21 nt, Homo sapiens
  source 1..21: mol_type = mRNA
aacactctgcccttcatcatc

SEQ ID NO: 1077 - RNA, 21 nt, Homo sapiens
  source 1..21: mol_type = mRNA
acactctgcccttcatcatca

SEQ ID NO: 1078 - RNA, 21 nt, Homo sapiens
  source 1..21: mol_type = mRNA
tctgcccttcatcatcacgat

SEQ ID NO: 1079 - RNA, 21 nt, Homo sapiens
  source 1..21: mol_type = mRNA
ctgcccttcatcatcacgatc

SEQ ID NO: 1080 - RNA, 21 nt, Homo sapiens
  source 1..21: mol_type = mRNA
tgcccttcatcatcacgatct

SEQ ID NO: 1081 - RNA, 21 nt, Homo sapiens
  source 1..21: mol_type = mRNA
cttcatcatcacgatcttctg

SEQ ID NO: 1082 - RNA, 21 nt, Homo sapiens
  source 1..21: mol_type = mRNA
ggccgccgctgcggaacctgt

SEQ ID NO: 1083 - RNA, 21 nt, Homo sapiens
  source 1..21: mol_type = mRNA
gccgccgctgcggaacctgtt

SEQ ID NO: 1084 - RNA, 21 nt, Homo sapiens
  source 1..21: mol_type = mRNA
ccgccgctgcggaacctgttc

SEQ ID NO: 1085 - RNA, 21 nt, Homo sapiens
  source 1..21: mol_type = mRNA
cgccgctgcggaacctgttca

SEQ ID NO: 1086 - RNA, 21 nt, Homo sapiens
  source 1..21: mol_type = mRNA
ggctggccaagcacgcgctgg

SEQ ID NO: 1087 - RNA, 21 nt, Homo sapiens
  source 1..21: mol_type = mRNA
gctggccaagcacgcgctgga

SEQ ID NO: 1088 - RNA, 21 nt, Homo sapiens
  source 1..21: mol_type = mRNA
ctggccaagcacgcgctggaa

SEQ ID NO: 1089 - RNA, 21 nt, Homo sapiens
  source 1..21: mol_type = mRNA
acgcgctggaaaacatgatta

SEQ ID NO: 1090 - RNA, 21 nt, Homo sapiens
  source 1..21: mol_type = mRNA
cacagtcagccatgttcaacc

SEQ ID NO: 1091 - RNA, 21 nt, Homo sapiens
  source 1..21: mol_type = mRNA
acagtcagccatgttcaacca

SEQ ID NO: 1092 - RNA, 21 nt, Homo sapiens
  source 1..21: mol_type = mRNA
cagtcagccatgttcaaccag

SEQ ID NO: 1093 - RNA, 21 nt, Homo sapiens
  source 1..21: mol_type = mRNA
agtcagccatgttcaaccagg

SEQ ID NO: 1094 - RNA, 21 nt, Homo sapiens
  source 1..21: mol_type = mRNA
tcagccatgttcaaccaggtc

SEQ ID NO: 1095 - RNA, 21 nt, Homo sapiens
  source 1..21: mol_type = mRNA
cagccatgttcaaccaggtcc

SEQ ID NO: 1096 - RNA, 21 nt, Homo sapiens
  source 1..21: mol_type = mRNA
agccatgttcaaccaggtcct

SEQ ID NO: 1097 - RNA, 21 nt, Homo sapiens
  source 1..21: mol_type = mRNA
gccatgttcaaccaggtcctc

SEQ ID NO: 1098 - RNA, 21 nt, Homo sapiens
  source 1..21: mol_type = mRNA
ttcaaccaggtcctcatcctc

SEQ ID NO: 1099 - RNA, 21 nt, Homo sapiens
  source 1..21: mol_type = mRNA
tcatcctcttctgcaccctgc

SEQ ID NO: 1100 - RNA, 21 nt, Homo sapiens
  source 1..21: mol_type = mRNA
tcctcttctgcaccctgctgt

SEQ ID NO: 1101 - RNA, 21 nt, Homo sapiens
  source 1..21: mol_type = mRNA
gtccctcctgacctccttcta

SEQ ID NO: 1102 - RNA, 21 nt, Homo sapiens
  source 1..21: mol_type = mRNA
ccctcctgacctccttctact

SEQ ID NO: 1103 - RNA, 21 nt, Homo sapiens
  source 1..21: mol_type = mRNA
ctcctgacctccttctacttc

SEQ ID NO: 1104 - RNA, 21 nt, Homo sapiens
  source 1..21: mol_type = mRNA
ctgacctccttctacttctgc

SEQ ID NO: 1105 - RNA, 21 nt, Homo sapiens
  source 1..21: mol_type = mRNA
ccttctacttctgcatcgtca

SEQ ID NO: 1106 - RNA, 21 nt, Homo sapiens
  source 1..21: mol_type = mRNA
cttctacttctgcatcgtcac

SEQ ID NO: 1107 - RNA, 21 nt, Homo sapiens
  source 1..21: mol_type = mRNA
tgctggtggtcatcatgatct

SEQ ID NO: 1108 - RNA, 21 nt, Homo sapiens
  source 1..21: mol_type = mRNA
cgaccttctcatggacttcct

SEQ ID NO: 1109 - RNA, 21 nt, Homo sapiens
  source 1..21: mol_type = mRNA
gaccttctcatggacttcctg

SEQ ID NO: 1110 - RNA, 21 nt, Homo sapiens
  source 1..21: mol_type = mRNA
ttctcatggacttcctgaacg

SEQ ID NO: 1111 - RNA, 21 nt, Homo sapiens
  source 1..21: mol_type = mRNA
tctcatggacttcctgaacga

SEQ ID NO: 1112 - RNA, 21 nt, Homo sapiens
  source 1..21: mol_type = mRNA
ctcatggacttcctgaacgag

SEQ ID NO: 1113 - RNA, 21 nt, Homo sapiens
  source 1..21: mol_type = mRNA
cccggctccaggactattacg

SEQ ID NO: 1114 - RNA, 21 nt, Homo sapiens
  source 1..21: mol_type = mRNA
caggactattacgtggtcatc

SEQ ID NO: 1115 - RNA, 21 nt, Homo sapiens
  source 1..21: mol_type = mRNA
tccagatcctcaaacctgaaa

SEQ ID NO: 1116 - RNA, 21 nt, Homo sapiens
  source 1..21: mol_type = mRNA
ctcaaacctgaaaacaagttt

SEQ ID NO: 1117 - RNA, 21 nt, Homo sapiens
  source 1..21: mol_type = mRNA
tcaaacctgaaaacaagtttc

SEQ ID NO: 1118 - RNA, 21 nt, Homo sapiens
  source 1..21: mol_type = mRNA
gtttgctgaccacgtggtgtg

SEQ ID NO: 1119 - RNA, 21 nt, Homo sapiens
  source 1..21: mol_type = mRNA
ctggcgctgaactgcatctgc

SEQ ID NO: 1120 - RNA, 21 nt, Homo sapiens
  source 1..21: mol_type = mRNA
gcatgggtgacagcaagttct

SEQ ID NO: 1121 - RNA, 21 nt, Homo sapiens
  source 1..21: mol_type = mRNA
catgggtgacagcaagttctt

SEQ ID NO: 1122 - RNA, 21 nt, Homo sapiens
  source 1..21: mol_type = mRNA
ggcgtgtgcctcatcgggctg

SEQ ID NO: 1123 - RNA, 21 nt, Homo sapiens
  source 1..21: mol_type = mRNA
tgtgcctcatcgggctgaagc

SEQ ID NO: 1124 - RNA, 21 nt, Homo sapiens
  source 1..21: mol_type = mRNA
gcctcatcgggctgaagcggg

SEQ ID NO: 1125 - RNA, 21 nt, Homo sapiens
  source 1..21: mol_type = mRNA
ctgacacctgcttctacatca

SEQ ID NO: 1126 - RNA, 21 nt, Homo sapiens
  source 1..21: mol_type = mRNA
cttctacatcaacatcaccaa

SEQ ID NO: 1127 - RNA, 21 nt, Homo sapiens
  source 1..21: mol_type = mRNA
ctgcggctggacaagggctgc

SEQ ID NO: 1128 - RNA, 21 nt, Homo sapiens
  source 1..21: mol_type = mRNA
gcctacgggttcaagaacaag

SEQ ID NO: 1129 - RNA, 21 nt, Homo sapiens
  source 1..21: mol_type = mRNA
cctacgggttcaagaacaagc

SEQ ID NO: 1130 - RNA, 21 nt, Homo sapiens
  source 1..21: mol_type = mRNA
ggttcaagaacaagctgatca

SEQ ID NO: 1131 - RNA, 21 nt, Homo sapiens
  source 1..21: mol_type = mRNA
gttcaagaacaagctgatcat

SEQ ID NO: 1132 - RNA, 21 nt, Homo sapiens
  source 1..21: mol_type = mRNA
ttcaagaacaagctgatcatc

SEQ ID NO: 1133 - RNA, 21 nt, Homo sapiens
  source 1..21: mol_type = mRNA
tgcgggcctactacagatccc

SEQ ID NO: 1134 - RNA, 21 nt, Homo sapiens
  source 1..21: mol_type = mRNA
gcgggcctactacagatcccg

SEQ ID NO: 1135 - RNA, 21 nt, Homo sapiens
  source 1..21: mol_type = mRNA
acaagcccgaccaccacttcc

SEQ ID NO: 1136 - RNA, 21 nt, Homo sapiens
  source 1..21: mol_type = mRNA
caagcccgaccaccacttcct

SEQ ID NO: 1137 - RNA, 21 nt, Homo sapiens
  source 1..21: mol_type = mRNA
aagcccgaccaccacttcctg

SEQ ID NO: 1138 - RNA, 21 nt, Homo sapiens
  source 1..21: mol_type = mRNA
agcccgaccaccacttcctgg

SEQ ID NO: 1139 - RNA, 21 nt, Homo sapiens
  source 1..21: mol_type = mRNA
ccgaccaccacttcctggaag

SEQ ID NO: 1140 - RNA, 21 nt, Homo sapiens
  source 1..21: mol_type = mRNA
cgaccaccacttcctggaagc

SEQ ID NO: 1141 - RNA, 21 nt, Homo sapiens
  source 1..21: mol_type = mRNA
gaccaccacttcctggaagcc

SEQ ID NO: 1142 - RNA, 21 nt, Homo sapiens
  source 1..21: mol_type = mRNA
gtggtggtggacaaggagagc

SEQ ID NO: 1143 - RNA, 21 nt, Homo sapiens
  source 1..21: mol_type = mRNA
ctctggctctttccaaactag

SEQ ID NO: 1144 - RNA, 21 nt, Homo sapiens
  source 1..21: mol_type = mRNA
tctggctctttccaaactaga

SEQ ID NO: 1145 - RNA, 21 nt, Homo sapiens
  source 1..21: mol_type = mRNA
ctggctctttccaaactagaa

SEQ ID NO: 1146 - RNA, 21 nt, Homo sapiens
  source 1..21: mol_type = mRNA
tggctctttccaaactagaaa

SEQ ID NO: 1147 - RNA, 21 nt, Homo sapiens
  source 1..21: mol_type = mRNA
ggctctttccaaactagaaaa

SEQ ID NO: 1148 - RNA, 21 nt, Homo sapiens
  source 1..21: mol_type = mRNA
ctttccaaactagaaaagagg

SEQ ID NO: 1149 - RNA, 21 nt, Homo sapiens
  source 1..21: mol_type = mRNA
ttccaaactagaaaagaggga

SEQ ID NO: 1150 - RNA, 21 nt, Homo sapiens
  source 1..21: mol_type = mRNA
tccaaactagaaaagagggag

SEQ ID NO: 1151 - RNA, 21 nt, Homo sapiens
  source 1..21: mol_type = mRNA
cctggccttcatgttccgcct

SEQ ID NO: 1152 - RNA, 21 nt, Homo sapiens
  source 1..21: mol_type = mRNA
ccttcatgttccgcctgccgt

SEQ ID NO: 1153 - RNA, 21 nt, Homo sapiens
  source 1..21: mol_type = mRNA
cttcatgttccgcctgccgtt

SEQ ID NO: 1154 - RNA, 21 nt, Homo sapiens
  source 1..21: mol_type = mRNA
ttcatgttccgcctgccgttc

SEQ ID NO: 1155 - RNA, 21 nt, Homo sapiens
  source 1..21: mol_type = mRNA
tcatgttccgcctgccgttcg

SEQ ID NO: 1156 - RNA, 21 nt, Homo sapiens
  source 1..21: mol_type = mRNA
ggccgcgtcttcagcatcagc

SEQ ID NO: 1157 - RNA, 21 nt, Homo sapiens
  source 1..21: mol_type = mRNA
gacacactgctctaccagtcc

SEQ ID NO: 1158 - RNA, 21 nt, Homo sapiens
  source 1..21: mol_type = mRNA
acacactgctctaccagtcct

SEQ ID NO: 1159 - RNA, 21 nt, Homo sapiens
  source 1..21: mol_type = mRNA
taccagtccttcgtgaaggac

SEQ ID NO: 1160 - RNA, 21 nt, Homo sapiens
  source 1..21: mol_type = mRNA
accagtccttcgtgaaggact

SEQ ID NO: 1161 - RNA, 21 nt, Homo sapiens
  source 1..21: mol_type = mRNA
cagtccttcgtgaaggactac

SEQ ID NO: 1162 - RNA, 21 nt, Homo sapiens
  source 1..21: mol_type = mRNA
agtccttcgtgaaggactaca

SEQ ID NO: 1163 - RNA, 21 nt, Homo sapiens
  source 1..21: mol_type = mRNA
gtccttcgtgaaggactacat

SEQ ID NO: 1164 - RNA, 21 nt, Homo sapiens
  source 1..21: mol_type = mRNA
ctacatgatcaccatcacccg

SEQ ID NO: 1165 - RNA, 21 nt, Homo sapiens
  source 1..21: mol_type = mRNA
tacatgatcaccatcacccgg

SEQ ID NO: 1166 - RNA, 21 nt, Homo sapiens
  source 1..21: mol_type = mRNA
acatgatcaccatcacccggc

SEQ ID NO: 1167 - RNA, 21 nt, Homo sapiens
  source 1..21: mol_type = mRNA
catgatcaccatcacccggct

SEQ ID NO: 1168 - RNA, 21 nt, Homo sapiens
  source 1..21: mol_type = mRNA
ctgtgccatgaaaatcaccga

SEQ ID NO: 1169 - RNA, 21 nt, Homo sapiens
  source 1..21: mol_type = mRNA
cagtcccagatctcggtgaac

SEQ ID NO: 1170 - RNA, 21 nt, Homo sapiens
  source 1..21: mol_type = mRNA
tctgagcgccaggagctctcc

SEQ ID NO: 1171 - RNA, 21 nt, Homo sapiens
  source 1..21: mol_type = mRNA
ccgagctggtgaagaaccgca

SEQ ID NO: 1172 - RNA, 21 nt, Homo sapiens
  source 1..21: mol_type = mRNA
ctacgacgagatgaacgacca

SEQ ID NO: 1173 - RNA, 21 nt, Homo sapiens
  source 1..21: mol_type = mRNA
ggtcctcaggaaggacgtgga

SEQ ID NO: 1174 - RNA, 21 nt, Homo sapiens
  source 1..21: mol_type = mRNA
gtggaggagcgtgtgaggaca

SEQ ID NO: 1175 - RNA, 21 nt, Homo sapiens
  source 1..21: mol_type = mRNA
ctcctgggactccaccctgga

SEQ ID NO: 1176 - RNA, 21 nt, Homo sapiens
  source 1..21: mol_type = mRNA
tcctgggactccaccctggaa

SEQ ID NO: 1177 - RNA, 21 nt, Homo sapiens
  source 1..21: mol_type = mRNA
cctgggactccaccctggaaa

SEQ ID NO: 1178 - RNA, 21 nt, Homo sapiens
  source 1..21: mol_type = mRNA
ctgggactccaccctggaaag

SEQ ID NO: 1179 - RNA, 21 nt, Homo sapiens
  source 1..21: mol_type = mRNA
tgcaggctagtgaggagagtt

SEQ ID NO: 1180 - RNA, 21 nt, Homo sapiens
  source 1..21: mol_type = mRNA
caggctagtgaggagagtttt

SEQ ID NO: 1181 - RNA, 21 nt, Homo sapiens
  source 1..21: mol_type = mRNA
tttacacgtcgatgcagtcca

SEQ ID NO: 1182 - RNA, 21 nt, Homo sapiens
  source 1..21: mol_type = mRNA
acttctctttacacagatgta

SEQ ID NO: 1183 - RNA, 21 nt, Homo sapiens
  source 1..21: mol_type = mRNA
ttctctttacacagatgtacc

SEQ ID NO: 1184 - RNA, 21 nt, Homo sapiens
  source 1..21: mol_type = mRNA
ccgcaactcgtgaccagggct

SEQ ID NO: 1185 - RNA, 21 nt, Homo sapiens
  source 1..21: mol_type = mRNA
tgtcgccgaggacctttctga

SEQ ID NO: 1186 - RNA, 21 nt, Homo sapiens
  source 1..21: mol_type = mRNA
gaaggaagcagaagacgccat

SEQ ID NO: 1187 - RNA, 21 nt, Homo sapiens
  source 1..21: mol_type = mRNA
gaagcagaagacgccatttcc

SEQ ID NO: 1188 - RNA, 21 nt, Homo sapiens
  source 1..21: mol_type = mRNA
aagcagaagacgccatttcct

SEQ ID NO: 1189 - RNA, 21 nt, Homo sapiens
  source 1..21: mol_type = mRNA
cagaagacgccatttcctcta

SEQ ID NO: 1190 - RNA, 21 nt, Homo sapiens
  source 1..21: mol_type = mRNA
agaagacgccatttcctctac

SEQ ID NO: 1191 - RNA, 21 nt, Homo sapiens
  source 1..21: mol_type = mRNA
agacgccatttcctctacttc

SEQ ID NO: 1192 - RNA, 21 nt, Homo sapiens
  source 1..21: mol_type = mRNA
acgccatttcctctacttcac

SEQ ID NO: 1193 - RNA, 21 nt, Homo sapiens
  source 1..21: mol_type = mRNA
ccatttcctctacttcacact

SEQ ID NO: 1194 - RNA, 21 nt, Homo sapiens
  source 1..21: mol_type = mRNA
tttcctctacttcacactgaa

SEQ ID NO: 1195 - RNA, 21 nt, Homo sapiens
  source 1..21: mol_type = mRNA
ttcctctacttcacactgaac

SEQ ID NO: 1196 - RNA, 21 nt, Homo sapiens
  source 1..21: mol_type = mRNA
ggcggaagatggtgcatttcc

SEQ ID NO: 1197 - RNA, 21 nt, Homo sapiens
  source 1..21: mol_type = mRNA
gcggaagatggtgcatttcca

SEQ ID NO: 1198 - RNA, 21 nt, Homo sapiens
  source 1..21: mol_type = mRNA
atggtgcatttccatggacca

SEQ ID NO: 1199 - RNA, 21 nt, Homo sapiens
  source 1..21: mol_type = mRNA
catggaccattttacacttac

SEQ ID NO: 1200 - RNA, 21 nt, Homo sapiens
  source 1..21: mol_type = mRNA
atggaccattttacacttacc

SEQ ID NO: 1201 - RNA, 21 nt, Homo sapiens
  source 1..21: mol_type = mRNA
tggaccattttacacttacct

SEQ ID NO: 1202 - RNA, 21 nt, Homo sapiens
  source 1..21: mol_type = mRNA
ggaccattttacacttacctt

SEQ ID NO: 1203 - RNA, 21 nt, Homo sapiens
  source 1..21: mol_type = mRNA
accattttacacttacctttt

SEQ ID NO: 1204 - RNA, 21 nt, Homo sapiens
  source 1..21: mol_type = mRNA
ccattttacacttacctttta

SEQ ID NO: 1205 - RNA, 21 nt, Homo sapiens
  source 1..21: mol_type = mRNA
cattttacacttaccttttaa

SEQ ID NO: 1206 - RNA, 21 nt, Homo sapiens
  source 1..21: mol_type = mRNA
attttacacttaccttttaaa

SEQ ID NO: 1207 - RNA, 21 nt, Homo sapiens
  source 1..21: mol_type = mRNA
ttttacacttaccttttaaag

SEQ ID NO: 1208 - RNA, 21 nt, Homo sapiens
  source 1..21: mol_type = mRNA
tacacttaccttttaaagcaa

SEQ ID NO: 1209 - RNA, 21 nt, Homo sapiens
  source 1..21: mol_type = mRNA
acacttaccttttaaagcaaa

SEQ ID NO: 1210 - RNA, 21 nt, Homo sapiens
  source 1..21: mol_type = mRNA
cacttaccttttaaagcaaag

SEQ ID NO: 1211 - RNA, 21 nt, Homo sapiens
  source 1..21: mol_type = mRNA
acttaccttttaaagcaaagc

SEQ ID NO: 1212 - RNA, 21 nt, Homo sapiens
  source 1..21: mol_type = mRNA
cttaccttttaaagcaaagcc

SEQ ID NO: 1213 - RNA, 21 nt, Homo sapiens
  source 1..21: mol_type = mRNA
taccttttaaagcaaagcctc

SEQ ID NO: 1214 - RNA, 21 nt, Homo sapiens
  source 1..21: mol_type = mRNA
cttttaaagcaaagcctcatt

SEQ ID NO: 1215 - RNA, 21 nt, Homo sapiens
  source 1..21: mol_type = mRNA
ttaaagcaaagcctcattttc

SEQ ID NO: 1216 - RNA, 21 nt, Homo sapiens
  source 1..21: mol_type = mRNA
tctcagcaaggcgagatcccg

SEQ ID NO: 1217 - RNA, 21 nt, Homo sapiens
  source 1..21: mol_type = mRNA
gccaccctgcgtcctttgctg

SEQ ID NO: 1218 - RNA, 21 nt, Homo sapiens
  source 1..21: mol_type = mRNA
tcctttgctgctggttcctta

SEQ ID NO: 1219 - RNA, 21 nt, Homo sapiens
  source 1..21: mol_type = mRNA
ttgctgctggttccttactgg

SEQ ID NO: 1220 - RNA, 21 nt, Homo sapiens
  source 1..21: mol_type = mRNA
tactggtttgtacggtcagcg

SEQ ID NO: 1221 - RNA, 21 nt, Homo sapiens
  source 1..21: mol_type = mRNA
tcagcgctggaaacttctatt

SEQ ID NO: 1222 - RNA, 21 nt, Homo sapiens
  source 1..21: mol_type = mRNA
cagcgctggaaacttctatta

SEQ ID NO: 1223 - RNA, 21 nt, Homo sapiens
  source 1..21: mol_type = mRNA
cgctggaaacttctattaaat

SEQ ID NO: 1224 - RNA, 21 nt, Homo sapiens
  source 1..21: mol_type = mRNA
ctattaaatggatgcattctg

(19) United States
(12) Patent Application Publication
(10) Pub. No.: US 2026/0250687 A1
(43) Pub. Date: Aug. 27, 2026

(54) COMPOSITIONS AND METHODS FOR TREATMENT OF EPILEPSIES

(71) Applicant: Atalanta Therapeutics, Inc., Boston, MA (US)

(72) Inventors: Stefan I. MCDONOUGH, Somerville, MA (US); Corrie GALLANT-BEHM, Wellesley, MA (US); Benjamin ANDREONE, Boston, MA (US); Matthew HASSLER, Boston, MA (US); Daniel CURTIS, Belmont, MA (US); Bruno Miguel Da Cruz GODINHO, Reading, MA (US)

(21) Appl. No.: 18/714,750

(22) PCT Filed: Dec. 1, 2022
(86) PCT No.: PCT/US2022/080766

§ 371 Date: May 30, 2024

Related U.S. Application Data
(60) Provisional application No. 63/284,932, filed on Dec. 1, 2021.

(51) Int. Cl. C12N 15/113; A61P 25/08

(52) U.S. Cl. CPC C12N 15/1138; A61P 25/08; C12N 2310/14; C12N 2310/315; C12N 2310/321

(57) ABSTRACT

The present disclosure provides single- or double-stranded interfering RNA molecules (e.g., siRNA) that target a sodium-activated potassium channel subfamily T member 1 (KCNT1) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having an epilepsy syndrome.

### TECHNICAL FIELD

This disclosure relates to small interfering RNA (SIRNA) molecules, and compositions containing the same, that target RNA transcripts (e.g., mRNA) of a sodium-activated potassium channel subfamily T member 1 (KCNT1) gene. The disclosure further describes methods for the treatment of epilepsy syndromes by delivering KCNT1-targeting siRNA molecules to the central nervous system of a subject in need.

### BACKGROUND

Epilepsy is a heterogeneous disorder caused by abnormalities in neuronal excitability and in the synchrony of neuronal firing. There are many mechanistically and symptomatically distinct forms of epilepsy. Genetic epilepsy syndromes can be incapacitating or even lethal in childhood, and existing first-line anticonvulsant treatments, such as zonisamide, phenytoin, mexiletine, lamotrigine, and carbamazepine, have little efficacy on the most severe syndromes.

One monogenetic driver of epilepsy is the sodium-activated potassium channel subfamily T member 1 (KCNT1) gene (also termed SLACK, K_{Na}1.1, and Slo2.2), one of over eighty homologous genes that encode a member of the voltage-gated potassium ion (K⁺) channel family. The full potassium channel protein is a tetramer that can be monomeric or heteromeric. Channels formed by KCNT1 are opened by depolarizing voltages and by intracellular calcium or sodium ions. KCNT1-driven epilepsy is poly-allelic in that many different individual mutations in KCNT1 can drive epilepsy, and there is not a single variant that causes most cases of the disease.

There remain challenges to making therapeutic inhibitors that reduce KCNT1 function by targeting KCNT1 protein. First, pathological effects of KCNT1 variants may be exerted not just by way of monomeric KCNT1 channels, but by way of channels incorporating polypeptide subunits encoded by other K⁺ channel genes, particularly other members of the KCa family. The target at the level of protein is not fully known, and an inhibitor of monomeric KCNT1 channel may not inhibit all pathological KCNT1 function. Second, over eighty genes encode subunits of K⁺ channels, and K⁺ channels drive critical cardiac, metabolic, and neurological functions. A successful therapeutic targeting KCNT1 would desirably exhibit high selectivity against K⁺ channel paralogs to avoid cardiac liabilities and neurological liabilities. Such selectivity has been challenging to achieve. Moreover, a successful therapeutic targeting KCNT1 would need to inhibit channels formed by most or all of the many KCNT1 alleles that drive epilepsy, and this may be a challenge for small molecules.

Accordingly, there remains a need for therapeutics capable of selectively diminishing KCNT1 activity among genes encoding other K⁺ channels in a manner that provides effective treatment for epilepsies.

### SUMMARY OF THE DISCLOSURE

The present disclosure provides compositions and methods for reduction of sodium-activated potassium channel subfamily T member 1 (KCNT1) expression by way of small interfering RNA (siRNA)-mediated silencing of sodium-activated potassium channel subfamily T member 1 (KCNT1) transcripts. The compositions and methods provide the benefit of exhibiting high selectivity toward KCNT1 over other central nervous system (CNS) genes, including those that encode other K⁺ channels.

The siRNA molecules of the disclosure can be used to silence the KCNT1 gene, thereby preventing the translation of the corresponding mRNA transcript and reducing KCNT1 expression. This reduction of KCNT1 levels thus prevents disease onset or progression. The siRNA molecules of the disclosure can be administered to pre-symptomatic individuals identified as having a gain-of-function KCNT1 mutation. The siRNA molecules of the disclosure can be delivered directly to the CNS of a subject in need of KCNT1 silencing by way of, for example, intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides an siRNA molecule containing an antisense strand and sense strand having complementarity to the antisense strand. The antisense strand has complementarity sufficient to hybridize to a region within a KCNT1 mRNA transcript having the nucleic acid sequence of any one of SEQ ID NOS: 437-654 and 1035-1224. In some embodiments, the antisense strand has complementarity sufficient to hybridize to a region within a KCNT1 mRNA transcript having the nucleic acid sequence of any one of SEQ ID NOs: 437-654. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 15 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 16 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 17 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 18 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 19 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 20 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 21 contiguous nucleobases within the KCNT1 mRNA transcript having the nucleic acid sequence of any one of SEQ ID NOs: 437-654 and 1035-1224. In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the KCNT1 mRNA transcript having the nucleic acid sequence of any one of SEQ ID NOs: 437-654 and 1035-1224.

In some embodiments, the antisense strand has at least 75% complementarity to the region within the KCNT1 mRNA transcript having the nucleic acid sequence of any one of SEQ ID NOs: 437-654 and 1035-1224. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the KCNT1 mRNA transcript having the nucleic acid sequence of any one of SEQ ID Nos: 437-654 and 1035-1224.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the KCNT1 mRNA transcript having the nucleic acid sequence of any one of SEQ ID Nos: 437-654 and 1035-1224.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the KCNT1 mRNA transcript having the nucleic acid sequence of any one of SEQ ID NOS: 437-654 and 1035-1224.

In some embodiments, the antisense strand has 21 contiguous nucleotides that are fully complementary to a contiguous polynucleotide segment of equal length within the region of the KCNT1 mRNA transcript having the nucleic acid sequence of any one of SEQ ID NOs: 437-654 and 1035-1224.

In some embodiments, the antisense strand has from 21 to 30 contiguous nucleotides (e.g., 21, 22, 23, 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID Nos: 437-654 and 1035-1224.

In some embodiments, the antisense strand has from 24 to 30 contiguous nucleotides (e.g., 24, 25, 26, 27, 28, 29, or 30 contiguous nucleotides) that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has 30 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

In some embodiments, the antisense strand has 9 or fewer nucleotide mismatches relative to the region of the KCNT1 RNA transcript having the nucleic acid sequence of any one of SEQ ID NOs: 437-654 and 1035-1224, optionally wherein the antisense strand contains 8 or fewer, 7 or fewer, 6 or fewer, 5 or fewer, 4 or fewer, 3 or fewer, 2 or fewer, or only 1 mismatch relative to the region of the KCNT1 RNA transcript having the nucleic acid sequence of any one of SEQ ID NOs: 437-654 and 1035-1224.

In some embodiments of any of the foregoing aspects or embodiments of the disclosure, the region of the KCNT1 RNA transcript has the nucleic acid sequence of any one of SEQ ID NOs: 437-654. In some embodiments of any of the foregoing aspects or embodiments of the disclosure, the region of the KCNT1 RNA transcript has the nucleic acid sequence of any one of SEQ ID NOs: 437-458, 460, 464-509, 522-560, 563-594, 598-603, 605-654, and 1035-1172. In some embodiments of any of the foregoing aspects or embodiments of the disclosure, the region of the KCNT1 RNA transcript has the nucleic acid sequence of SEQ ID NO: 455.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 1-218 and 655-844. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 1-218. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 1-22, 24, 28-73, 86-124, 127-158, 162-167, 169-218, and 655-792. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 19.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOS: 1-218 and 655-844. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 1-218.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 1-22, 24, 28-73, 86-124, 127-158, 162-167, 169-218, and 655-792. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 19.

In some embodiments, the antisense strand has a nucleic acid sequence that is at least 95% identical (e.g., 95%, 96%, 97%, 98%, 99%, or 100% identical) to the nucleic acid sequence of SEQ ID NOs: 1-218 and 655-844, optionally wherein the antisense strand has a nucleic acid sequence that is at least 96%, 97%, 98%, or 99% identical to the nucleic acid sequence of any one of SEQ ID NOs: 1-218 and 655-844. In some embodiments, the antisense strand has a nucleic acid sequence that is at least 95% identical (e.g., 95%, 96%, 97%, 98%, 99%, or 100% identical) to the nucleic acid sequence of SEQ ID NOs: 1-218, optionally wherein the antisense strand has a nucleic acid sequence that is at least 96%, 97%, 98%, or 99% identical to the nucleic acid sequence of any one of SEQ ID NOs: 1-218. In some embodiments, the antisense strand has a nucleic acid sequence that is at least 95% identical (e.g., 95%, 96%, 97%, 98%, 99%, or 100% identical) to the nucleic acid sequence of SEQ ID NOs: 1-22, 24, 28-73, 86-124, 127-158, 162-167, 169-218, and 655-79, optionally wherein the antisense strand has a nucleic acid sequence that is at least 96%, 97%, 98%, or 99% identical to the nucleic acid sequence of any one of SEQ ID NOS: 1-22, 24, 28-73, 86-124, 127-158, 162-167, 169-218, and 655-79. In some embodiments, the antisense strand has a nucleic acid sequence that is at least 95% identical (e.g., 95%, 96%, 97%, 98%, 99%, or 100% identical) to the nucleic acid sequence of SEQ ID NO: 19, optionally wherein the antisense strand has a nucleic acid sequence that is at least 96%, 97%, 98%, or 99% identical to the nucleic acid sequence of SEQ ID NO: 19.

In some embodiments, the antisense strand has the nucleic acid sequence of any one of SEQ ID NOs: 1-218 and 655-844. In some embodiments, the antisense strand has the nucleic acid sequence of any one of SEQ ID NOs: 1-218. In some embodiments, the antisense strand has the nucleic acid sequence of any one of SEQ ID NOs: 1-22, 24, 28-73, 86-124, 127-158, 162-167, 169-218, and 655-79. In some embodiments, the antisense strand has the nucleic acid sequence of SEQ ID NO: 19.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 219-436 and 845-1034. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 219-436. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 219-240, 242, 246-291, 304-342, 345-376, 380-385, 387-436, and 845-982. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of SEQ ID NO: 237.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOS: 219-436 and 845-1034. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 219-436. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 219-240, 242, 246-291, 304-342, 345-376, 380-385, 387-436, and 845-982. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of SEQ ID NO: 237.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 219-436 and 845-1034, optionally wherein the sense strand has a nucleic acid sequence that is at least 96%, 97%, 98%, or 99% identical to the nucleic acid sequence of any one of SEQ ID NOs: 219-436 and 845-1034. In some embodiments, the sense strand has a nucleic acid sequence that is at least 95% identical (e.g., 95%, 96%, 97%, 98%, 99%, or 100% identical) to the nucleic acid sequence of any one of SEQ ID NOS: 219-436, optionally wherein the sense strand has a nucleic acid sequence that is at least 96%, 97%, 98%, or 99% identical to the nucleic acid sequence of any one of SEQ ID NOs: 219-436. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 219-240, 242, 246-291, 304-342, 345-376, 380-385, 387-436, and 845-982, optionally wherein the sense strand has a nucleic acid sequence that is at least 96%, 97%, 98%, or 99% identical to the nucleic acid sequence of any one of SEQ ID NOs: 219-240, 242, 246-291, 304-342, 345-376, 380-385, 387-436, and 845-982. In some embodiments, the sense strand has a nucleic acid sequence that is at least 95% identical (e.g., 95%, 96%, 97%, 98%, 99%, or 100% identical) to the nucleic acid sequence of SEQ ID NO: 237, optionally wherein the sense strand has a nucleic acid sequence that is at least 96%, 97%, 98%, or 99% identical to the nucleic acid sequence of any one of SEQ ID NO: 237.

In some embodiments, the siRNA molecule has a sense strand having the nucleic acid sequence of any one of SEQ ID NOs: 219-436 and 845-1034. In some embodiments, the siRNA molecule has a sense strand having the nucleic acid sequence of any one of SEQ ID NOs: 219-436. In some embodiments, the siRNA molecule has a sense strand having the nucleic acid sequence of any one of SEQ ID NOS: 219-240, 242, 246-291, 304-342, 345-376, 380-385, 387-436, and 845-982. In some embodiments, the siRNA molecule has a sense strand having the nucleic acid sequence of SEQ ID NO: 237.

In some embodiments, the antisense strand has a structure represented by Formula I, wherein Formula I is, in the 5′- to -3′ direction:

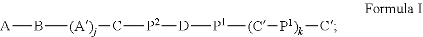

wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′- to -3′ direction:

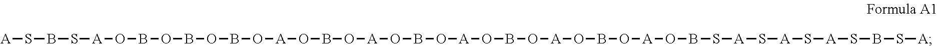

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′- to -3′ direction:

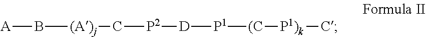

wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′- to -3′ direction:

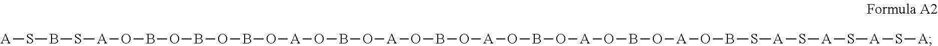

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′- to -3′ direction:

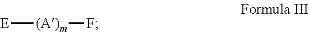

wherein E is represented by the formula (C-P¹)₂;
F is represented by the formula (C-P²) 3-D-P¹-C-P¹-C, (C-P²) 3-D-P²-C-P²-C, (C-P²) 3-D-P¹-C-P¹-D, or (C-P²) 3-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′- to -3′ direction:

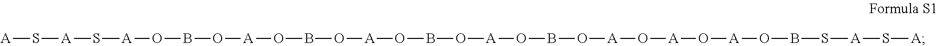

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′- to -3′ direction:

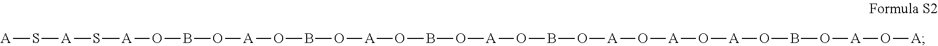

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′- to -3′ direction:

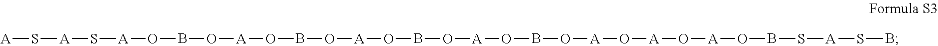

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′- to -3′ direction:

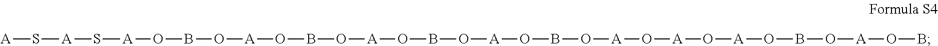

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′- to -3′ direction:

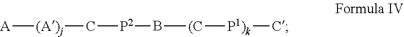

wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′- to -3′ direction:

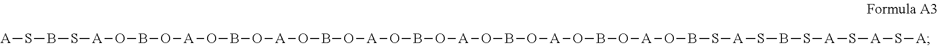

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′- to -3′ direction:

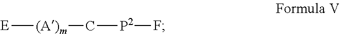

wherein E is represented by the formula (C-P¹)₂; F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′- to -3′ direction:

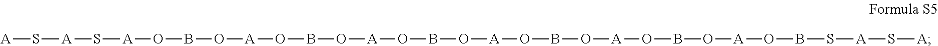

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′- to -3′ direction:

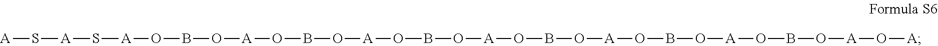

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′- to -3′ direction:

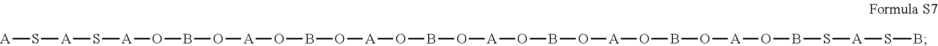

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′- to -3′ direction:

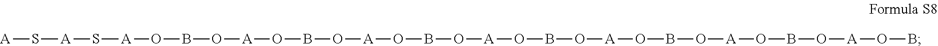

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′- to -3′ direction:

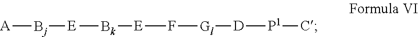

wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′- to -3′ direction:

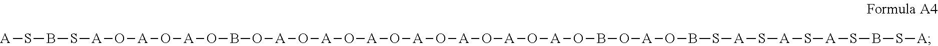

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′- to -3′ direction:

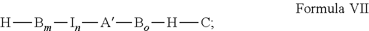

wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′- to -3′ direction:

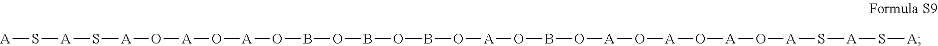

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

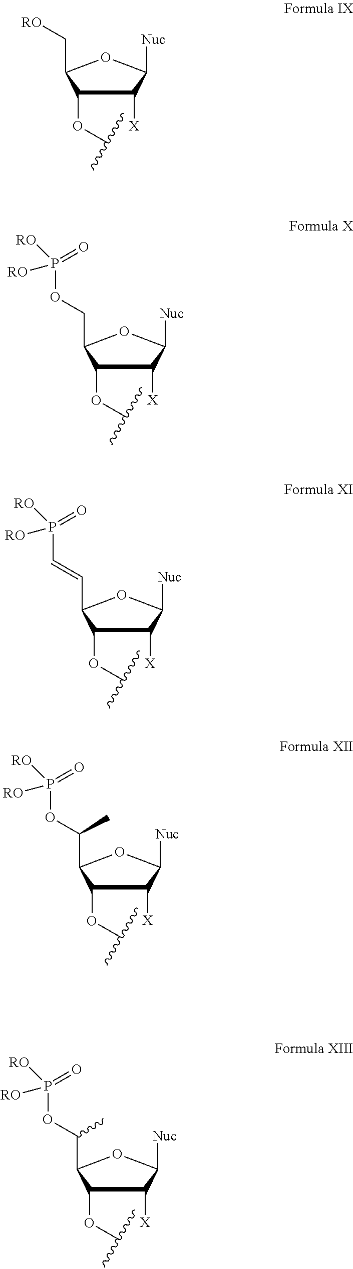

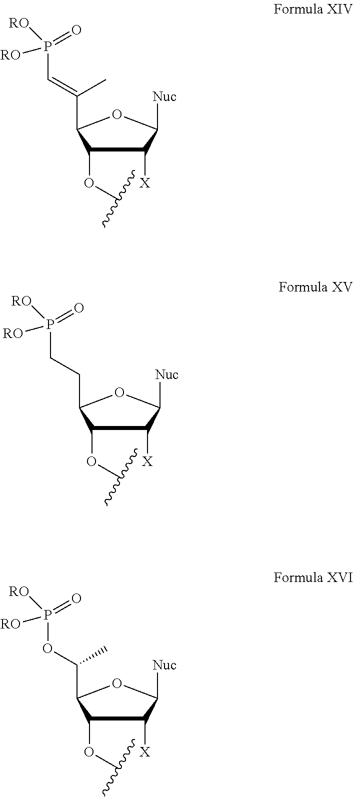

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

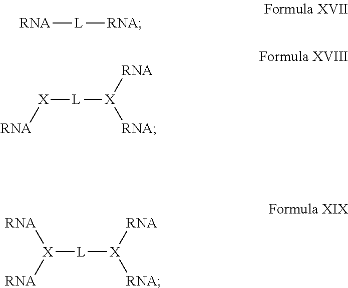

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

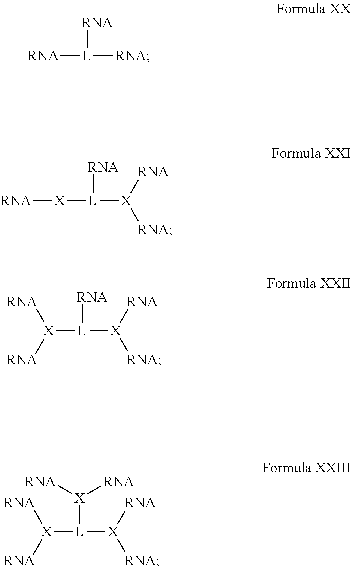

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched SiRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

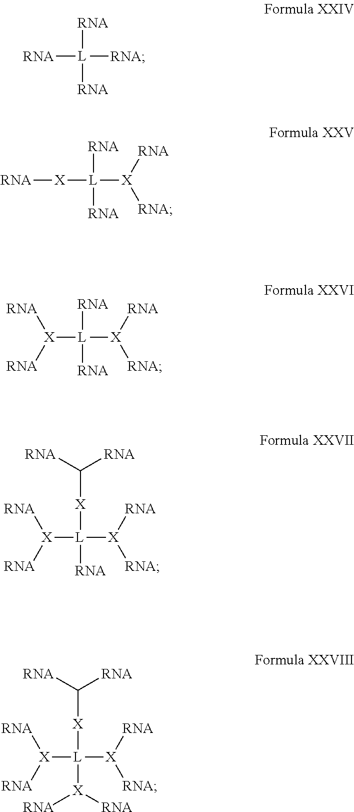

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

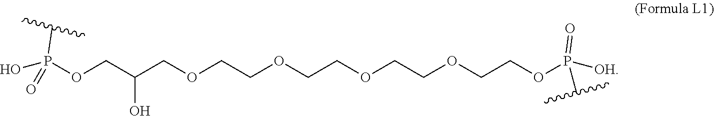 [SMILES: *P(=O)(O)OCCOCCOCCOCCOCC(O)COP(*)(=O)O]

In some embodiments, the linker includes a structure of Formula L2:

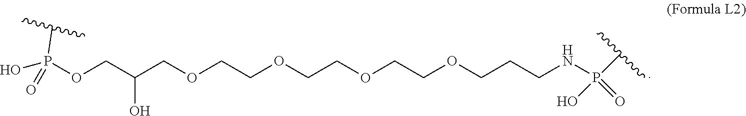 [SMILES: *P(=O)(O)NCCCOCCOCCOCCOCC(O)COP(*)(=O)O]

In some embodiments, the linker includes a structure of Formula L3:

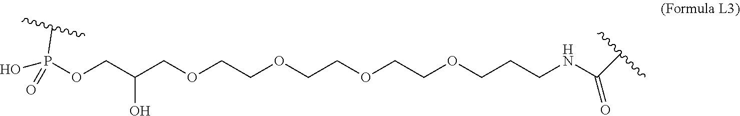 [SMILES: *C(=O)NCCCOCCOCCOCCOCC(O)COP(*)(=O)O]

In some embodiments, the linker includes a structure of Formula L4:

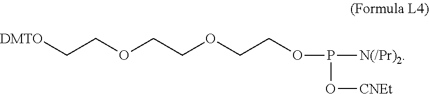

In some embodiments, the linker includes a structure of Formula L5:

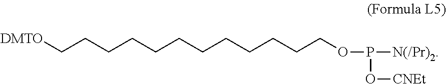

In some embodiments, the linker includes a structure of Formula L6:

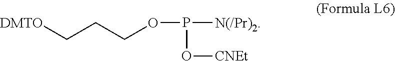

In some embodiments, the linker includes a structure of Formula L7:

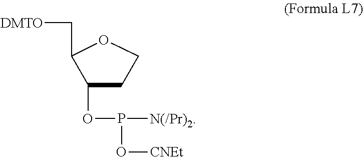

In some embodiments, the linker includes a structure of Formula L8:

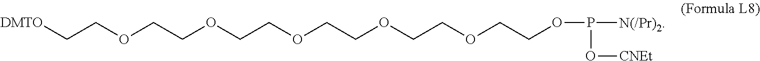

In some embodiments, the linker includes a structure of Formula L9:

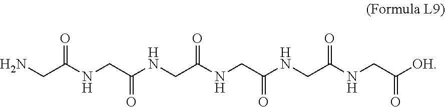 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to the CNS of a subject diagnosed as having an epilepsy syndrome by administering a therapeutically effective amount of the siRNA molecule or a pharmaceutical composition of any of the preceding aspects or embodiments of the disclosure to the subject.

In a further aspect, the disclosure provides a method of treating an epilepsy syndrome in a subject in need thereof by administering a therapeutically effective amount of an siRNA molecule or a pharmaceutical composition of any of the preceding aspects or embodiments of the disclosure to the CNS of the subject.

In some embodiments, the epilepsy syndrome is frontal lobe epilepsy, occipital lobe epilepsy, medial temporal lobe epilepsy, parietal lobe epilepsy, benign myoclonic epilepsy in infants, juvenile myoclonic epilepsy, childhood absence epilepsy, juvenile absence epilepsy, epilepsy with generalized tonic clonic seizures in childhood, infantile spasms, Lennox-Gastaut syndrome, West syndrome, sleep-related hypermotor epilepsy, progressive myoclonus epilepsies, febrile fits, epilepsy with continuous spike and waves in slow wave sleep, Laudau Kleffner syndrome, Rasmussen's syndrome, epilepsy arising from an inborn error in metabolism, epilepsy of infancy with migrating focal seizures, autosomal dominant nocturnal frontal lobe epilepsy, Ohtahara syndrome, early myoclonic encephalopathy, focal epilepsy, and/or multifocal epilepsy.

In another aspect, the disclosure provides a method of reducing KCNT1 expression in a subject in need thereof by administering a therapeutically effective amount of an siRNA or pharmaceutical composition of any of the preceding aspects or embodiments of the disclosure.

In some embodiments, the subject exhibits selective reduction in KCNT1 expression compared to reduction in expression of one or more other voltage-gated potassium ion channels upon administration of an siRNA molecule or pharmaceutical composition of any of the preceding aspects or embodiments of the disclosure.

In some embodiments, the siRNA molecule or the pharmaceutical composition is administered to the subject by way of intracerebroventricular or intrathecal injection.

In some embodiments, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### BRIEF DESCRIPTION OF THE FIGURE

FIG. 1 is a graph showing the IC50 determination of an exemplary siRNA molecule of the disclosure having an antisense strand of SEQ ID NO: 19 and a sense strand of SEQ ID NO: 237. The IC50 was determined to be 0.0415 nM.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (SiRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of KCNT1, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C═C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl —CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that experiences episodes of epilepsy and/or epileptic-associated phenotypes (e.g., seizures, perioral cyanosis, flushing, apnea, hypertonia, pulmonary hemorrhage, cardiac arrythmia, etc.) and/or contains a gain-of-function KCNT1 variant allele. Nonlimiting examples of subjects and patients diagnosed with epilepsy include those diagnosed with epilepsy of infancy with migrating focal seizures (EIMFS), autosomal dominant nocturnal frontal lobe epilepsy (ADNFLE), West syndrome, Ohtahara syndrome, early myoclonic encephalopathy, leukodystrophy and/or leukoencephalopathy, focal epilepsy, and multifocal epilepsy.

As used herein, the term “epilepsy” refers to any of a variety of types of epilepsy syndromes, including, but not limited to, frontal lobe epilepsy, occipital lobe epilepsy, medial temporal lobe epilepsy, parietal lobe epilepsy, benign myoclonic epilepsy in infants, juvenile myoclonic epilepsy, childhood absence epilepsy, juvenile absence epilepsy, epilepsy with generalized tonic clonic seizures in childhood, infantile spasms, Lennox-Gastaut syndrome, West syndrome, sleep-related hypermotor epilepsy, progressive myoclonus epilepsies, febrile fits, epilepsy with continuous spike and waves in slow wave sleep, Laudau Kleffner syndrome, Rasmussen's syndrome, epilepsy arising from an inborn error in metabolism, epilepsy of infancy with migrating focal seizures, autosomal dominant nocturnal frontal lobe epilepsy, Ohtahara syndrome, early myoclonic encephalopathy, focal epilepsy, and/or multifocal epilepsy.

As used herein, the term “KCNT1” refers to the gene encoding the sodium-activated potassium channel subfamily T member 1 (also termed SLACK, K_{Na}1.1, and Slo2.2), including any native KCNT1 gene from any source. The term encompasses “full-length,” unprocessed KCNT1 as well as any form of KCNT1 that results from processing in the cell. The term also encompasses naturally occurring variants of KCNT1, e.g., splice variants or allelic variants. The nucleic acid sequence of an exemplary KCNT1 gene is shown in European Nucleotide Archive (ENA) Accession No. KP869068.1. The amino acid sequence of an exemplary protein encoded by a KCNT1 gene is shown in UNIPROT™ Accession No. Q5JUK3.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on anticonvulsant treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, epilepsy. For example, clinical benefits in the context of a subject administered an siRNA molecule or siRNA composition of the disclosure include, without limitation, a reduced dependency or total withdrawal of anticonvulsant, sedative, and/or nerve pain medication, and/or; a reduction in the duration and/or frequency of epileptic events experienced by the subject, and/or; a reduction in epileptic-associated phenotypes (e.g., seizures, perioral cyanosis, flushing, apnea, hypertonia, pulmonary hemorrhage, cardiac arrythmia, etc.), and/or; a reduction in wild type KCNT1 transcripts, mutant KCNT1 transcripts, variant KCNT1 transcripts, splice isoforms of KCNT1 transcripts, and/or overexpressed KCNT1 transcripts.

### DETAILED DESCRIPTION

The present disclosure provides compositions of small interfering RNA (siRNA) molecules with sequence homology to a sodium-activated potassium channel subfamily T member 1 (KCNT1) gene and methods for administering said siRNA molecules to the central nervous system of a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of KCNT1, relative to other genes in the voltage-gated potassium ion (K⁺) channel family. In particular, none of the oligonucleotides of the disclosure contain a seed siRNA sequence that matches closely related K⁺ channel genes KCNT2 and KCNMA1, the cardiac KCNH2, KCNQ1, KCNE1, and KCNE2 genes, or genes of the KCNA, KCNB, KCNC, KCND, KCNQ, KCNV, or KCNH families, which are associated with adverse neurological or other outcomes, usually by way of loss-of-function genetic variants.

The siRNA molecules of the disclosure may feature an antisense strand having a nucleic acid sequence that is complementary to a region of a KCNT1 mRNA transcript having the nucleic acid sequence of any one of SEQ ID NOs: 437-654 and 1035-1224. The degree of complementarity of the antisense strand to the region of the KCNT1 mRNA transcript may be sufficient for the antisense strand to anneal over the full length of the region of the KCNT1 mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the KCNT1 mRNA transcript.

In some embodiments, the siRNA molecules of the disclosure feature an antisense strand having the nucleic acid sequence of any one of SEQ ID NOS: 1-218 and 655-844, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 1-218 and 655-844.

In some embodiments, the siRNA molecules of the disclosure feature a sense strand having the nucleic acid sequence of any one of SEQ ID NOS: 219-436 and 845-1034, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 219-436 and 845-1034.

Exemplary siRNA molecules of the disclosure are those shown in Table 1, below. Table 1 summarizes the antisense strands, sense strands, and corresponding regions of a KCNT1 mRNA transcript that are targeted by each antisense strand.

TABLE 1
Nucleotide sequences for gene-specific KCNT1-targeting siRNA

| | | | | Targeting | KCNT1 mRNA |
|---|---|---|---|---|---|
| Antisense | Antisense | Sense SEQ ID | | Region SEQ | targeted |
| SEQ ID NO: | Strand | NO: | Sense Strand | ID NO: | sequence |
| 1 | UACAGCUUG | 219 | AGCGGCUCA | 437 | CAAGGAGCG |
| | AGCCGCUCC | | AGCUGUA | | GCUCAAGCU |
| | UUG | | | | GUU |
| 2 | UAACAGCUU | 220 | GCGGCUCAA | 438 | AAGGAGCGG |
| | GAGCCGCUC | | GCUGUUA | | CUCAAGCUG |
| | CUU | | | | UUC |
| 3 | UAGAACAGC | 221 | GGCUCAAGC | 439 | GGAGCGGC |
| | UUGAGCCGC | | UGUUCUA | | UCAAGCUGU |
| | UCC | | | | UCUU |
| 4 | UAAGAACAG | 222 | GCUCAAGCU | 440 | GAGCGGCUC |
| | CUUGAGCCG | | GUUCUUA | | AAGCUGUUC |
| | CUC | | | | UUC |
| 5 | UGAAGAACA | 223 | CUCAAGCUG | 441 | AGCGGCUCA |
| | GCUUGAGCC | | UUCUUCA | | AGCUGUUCU |
| | GCU | | | | UCA |
| 6 | UUGAAGAAC | 224 | UCAAGCUGU | 442 | GCGGCUCAA |
| | AGCUUGAGC | | UCUUCAA | | GCUGUUCUU |
| | CGC | | | | CAU |
| 7 | UAUGAAGAA | 225 | CAAGCUGUU | 443 | CGGCUCAAG |
| | CAGCUUGAG | | CUUCAUA | | CUGUUCUUC |
| | CCG | | | | AUC |
| 8 | UGAUGAAGA | 226 | AAGCUGUUC | 444 | GGCUCAAGC |
| | ACAGCUUGA | | UUCAUCA | | UGUUCUUCA |
| | GCC | | | | UCA |
| 9 | UUGAUGAAG | 227 | AGCUGUUCU | 445 | GCUCAAGCU |
| | AACAGCUUG | | UCAUCAA | | GUUCUUCAU |
| | AGC | | | | CAA |
| 10 | UUUGAUGAA | 228 | GCUGUUCUU | 446 | CUCAAGCUG |
| | GAACAGCUU | | CAUCAAA | | UUCUUCAUC |
| | GAG | | | | AAA |
| 11 | UUUUGAUGA | 229 | CUGUUCUUC | 447 | UCAAGCUGU |
| | AGAACAGCU | | AUCAAAA | | UCUUCAUCA |
| | UGA | | | | AAA |
| 12 | UUGAACAGC | 230 | GGAUCCGGC | 448 | CCUGAGGAU |
| | CGGAUCCUC | | UGUUCAA | | CCGGCUGUU |
| | AGG | | | | CAA |
| 13 | UUUGAACAG | 231 | GAUCCGGCU | 449 | CUGAGGAUC |
| | CCGGAUCCU | | GUUCAAA | | CGGCUGUUC |
| | CAG | | | | AAC |
| 14 | UGUUGAACA | 232 | AUCCGGCUG | 450 | UGAGGAUCC |
| | GCCGGAUCC | | UUCAACA | | GGCUGUUCA |
| | UCA | | | | ACU |
| 15 | UAGUUGAAC | 233 | UCCGGCUGU | 451 | GAGGAUCCG |
| | AGCCGGAUC | | UCAACUA | | GCUGUUCAA |
| | CUC | | | | CUU |
| 16 | UAAGUUGAA | 234 | CCGGCUGUU | 452 | AGGAUCCGG |
| | CAGCCGGAU | | CAACUUA | | CUGUUCAAC |
| | CCU | | | | UUC |
| 17 | UGAAGUUGA | 235 | CGGCUGUUC | 453 | GGAUCCGGC |
| | ACAGCCGGA | | AACUUCA | | UGUUCAACU |
| | UCC | | | | UCU |
| 18 | UAGAAGUUG | 236 | GGCUGUUCA | 454 | GAUCCGGCU |
| | AACAGCCGG | | ACUUCUA | | GUUCAACUU |
| | AUC | | | | CUC |
| 19 | UGAGAAGUU | 237 | GCUGUUCAA | 455 | AUCCGGCUG |
| | GAACAGCCG | | CUUCUCA | | UUCAACUUC |
| | GAU | | | | UCC |
| 20 | UGGAGAAGU | 238 | CUGUUCAAC | 456 | UCCGGCUGU |
| | UGAACAGCC | | UUCUCCA | | UCAACUUCU |
| | GGA | | | | CCC |
| 21 | UGGGAGAAG | 239 | UGUUCAACU | 457 | CCGGCUGUU |
| | UUGAACAGC | | UCUCCCA | | CAACUUCUC |
| | CGG | | | | CCU |
| 22 | UAGGGAGAA | 240 | GUUCAACUU | 458 | CGGCUGUUC |
| | GUUGAACAG | | CUCCCUA | | AACUUCUCC |
| | CCG | | | | CUG |
| 23 | UCCAGCAUC | 241 | GGCAUCGGA | 459 | CCCUGGGCA |
| | CGAUGCCCA | | UGCUGGA | | UCGGAUGCU |
| | GGG | | | | GGG |
| 24 | UCCCAGCAU | 242 | GCAUCGGAU | 460 | CCUGGGCAU |
| | CCGAUGCCC | | GCUGGGA | | CGGAUGCUG |
| | AGG | | | | GGG |
| 25 | UCUUUGUAG | 243 | ACCUCAGCU | 46 | CAUCUACCU |
| | CUGAGGUAG | | ACAAAGA | | CAGCUACAA |
| | AUG | | | | AGG |
| 26 | UCCUUUGUA | 244 | CCUCAGCUA | 462 | AUCUACCUC |
| | GCUGAGGUA | | CAAAGGA | | AGCUACAAA |
| | GAU | | | | GGC |
| 27 | UGUUGCCUU | 245 | AGCUACAAA | 463 | ACCUCAGCU |
| | UGUAGCUGA | | GGCAACA | | ACAAAGGCA |
| | GGU | | | | ACA |
| 28 | UUGUUGCCU | 246 | GCUACAAAG | 464 | CCUCAGCUA |
| | UUGUAGCUG | | GCAACAA | | CAAAGGCAA |
| | AGG | | | | CAU |
| 29 | UAUGUUGCC | 247 | CUACAAAGG | 465 | CUCAGCUAC |
| | UUUGUAGCU | | CAACAUA | | AAAGGCAAC |
| | GAG | | | | AUC |
| 30 | UGAUGUUGC | 248 | UACAAAGGC | 466 | UCAGCUACA |
| | CUUUGUAGC | | AACAUCA | | AAGGCAACA |
| | UGA | | | | UCU |
| 31 | UAGAUGUUG | 249 | ACAAAGGCA | 467 | CAGCUACAA |
| | CCUUUGUAG | | ACAUCUA | | AGGCAACAU |
| | CUG | | | | CUG |
| 32 | UCAGAUGUU | 250 | CAAAGGCAA | 468 | AGCUACAAA |
| | GCCUUUGUA | | CAUCUGA | | GGCAACAUC |
| | GCU | | | | UGG |
| 33 | UCCAGAUGU | 251 | AAAGGCAAC | 469 | GCUACAAAG |
| | UGCCUUUGU | | AUCUGGA | | GCAACAUCU |
| | AGC | | | | GGG |
| 34 | UCCCAGAUG | 252 | AAGGCAACA | 470 | CUACAAAGG |
| | UUGCCUUUG | | UCUGGGA | | CAACAUCUG |
| | UAG | | | | GGA |
| 35 | UUCCCAGAU | 253 | AGGCAACAU | 471 | UACAAAGGC |
| | GUUGCCUUU | | CUGGGAA | | AACAUCUGG |
| | GUA | | | | GAG |
| 36 | UCUCCCAGA | 254 | GGCAACAUC | 472 | ACAAAGGCA |
| | UGUUGCCUU | | UGGGAGA | | ACAUCUGGG |
| | UGU | | | | AGC |
| 37 | UGCUCCCAG | 255 | GCAACAUCU | 473 | CAAAGGCAA |
| | AUGUUGCCU | | GGGAGCA | | CAUCUGGGA |
| | UUG | | | | GCA |
| 38 | UGUGUUGAU | 256 | GGAGAUGAU | 474 | GUCCUGGAG |
| | CAUCUCCAG | | CAACACA | | AUGAUCAAC |
| | GAC | | | | ACU |
| 39 | UUGAUGAUG | 257 | UGCCCUUCA | 475 | CACUCUGCC |
| | AAGGGCAGA | | UCAUCAA | | CUUCAUCAU |
| | GUG | | | | CAC |
| 40 | UGUGAUGAU | 258 | GCCCUUCAU | 476 | ACUCUGCCC |
| | GAAGGGCAG | | CAUCACA | | UUCAUCAUC |
| | AGU | | | | ACG |
| 41 | UCGUGAUGA | 259 | CCCUUCAUC | 477 | CUCUGCCCU |
| | UGAAGGGCA | | AUCACGA | | UCAUCAUCA |
| | GAG | | | | CGA |
| 42 | UUGAACAGG | 260 | UGCGGAACC | 478 | GCCGCUGC |
| | UUCCGCAGC | | UGUUCAA | | GGAACCUGU |
| | GGC | | | | UCAU |
| 43 | UAUGAACAG | 261 | GCGGAACCU | 479 | CCGCUGCG |
| | GUUCCGCAG | | GUUCAUA | | GAACCUGUU |
| | CGG | | | | CAUC |
| 44 | UGAUGAACA | 262 | CGGAACCUG | 480 | CGCUGCGGA |
| | GGUUCCGCA | | UUCAUCA | | ACCUGUUCA |
| | GCG | | | | UCC |
| 45 | UGGAUGAAC | 263 | GGAACCUGU | 481 | GCUGCGGAA |
| | AGGUUCCGC | | UCAUCCA | | CCUGUUCAU |
| | AGC | | | | CCC |
| 46 | UGGGAUGAA | 264 | GAACCUGUU | 482 | CUGCGGAAC |
| | CAGGUUCCG | | CAUCCCA | | CUGUUCAUC |
| | CAG | | | | CCC |
| 47 | UUGGCACGG | 265 | ACUUCCACC | 483 | UAAUGACUU |
| | UGGAAGUCA | | GUGCCAA | | CCACCGUGC |
| | UUA | | | | CAU |
| 48 | UAUGGCACG | 266 | CUUCCACCG | 484 | AAUGACUUC |
| | GUGGAAGUC | | UGCCAUA | | CACCGUGCC |
| | AUU | | | | AUC |
| 49 | UGAUGGCAC | 267 | UUCCACCGU | 485 | AUGACUUCC |
| | GGUGGAAGU | | GCCAUCA | | ACCGUGCCA |
| | CAU | | | | UCC |
| 50 | UGGAUGGCA | 268 | UCCACCGUG | 486 | UGACUUCCA |
| | CGGUGGAAG | | CCAUCCA | | CCGUGCCAU |
| | UCA | | | | CCU |
| 51 | UAGGAUGGC | 269 | CCACCGUGC | 487 | GACUUCCAC |
| | ACGGUGGAA | | CAUCCUA | | CGUGCCAUC |
| | GUC | | | | CUG |
| 52 | UCCUGGUUG | 270 | CCAUGUUCA | 488 | GUCAGCCAU |
| | AACAUGGCU | | ACCAGGA | | GUUCAACCA |
| | GAC | | | | GGU |
| 53 | UACAGCAGG | 27 | UCUGCACCC | 489 | CCUCUUCUG |
| | GUGCAGAAG | | UGCUGUA | | CACCCUGCU |
| | AGG | | | | GUG |
| 54 | UGGCACAGC | 272 | GCACCCUGC | 490 | CUUCUGCAC |
| | AGGGUGCAG | | UGUGCCA | | CCUGCUGUG |
| | AAG | | | | CCU |
| 55 | UAGUAGAAG | 273 | UGACCUCCU | 491 | CCUCCUGAC |
| | GAGGUCAGG | | UCUACUA | | CUCCUUCUA |
| | AGG | | | | CUU |
| 56 | UGAAGUAGA | 274 | ACCUCCUUC | 492 | UCCUGACCU |
| | AGGAGGUCA | | UACUUCA | | CCUUCUACU |
| | GGA | | | | UCU |
| 57 | UAGAAGUAG | 275 | CCUCCUUCU | 493 | CCUGACCUC |
| | AAGGAGGUC | | ACUUCUA | | CUUCUACUU |
| | AGG | | | | CUG |
| 58 | UGCAGAAGU | 276 | UCCUUCUAC | 494 | UGACCUCCU |
| | AGAAGGAGG | | UUCUGCA | | UCUACUUCU |
| | UCA | | | | GCA |
| 59 | UUGCAGAAG | 277 | CCUUCUACU | 495 | GACCUCCUU |
| | UAGAAGGAG | | UCUGCAA | | CUACUUCUG |
| | GUC | | | | CAU |
| 60 | UAUGCAGAA | 278 | CUUCUACUU | 496 | ACCUCCUUC |
| | GUAGAAGGA | | CUGCAUA | | UACUUCUGC |
| | GGU | | | | AUC |
| 61 | UGAUGCAGA | 279 | UUCUACUUC | 497 | CCUCCUUCU |
| | AGUAGAAGG | | UGCAUCA | | ACUUCUGCA |
| | AGG | | | | UCG |
| 62 | UCGAUGCAG | 280 | UCUACUUCU | 498 | CUCCUUCUA |
| | AAGUAGAAG | | GCAUCGA | | CUUCUGCAU |
| | GAG | | | | CGU |
| 63 | UACGAUGCA | 281 | CUACUUCUG | 499 | UCCUUCUAC |
| | GAAGUAGAA | | CAUCGUA | | UUCUGCAUC |
| | GGA | | | | GUC |
| 64 | UGCCAGAUC | 282 | CGCCCAAGA | 500 | CGUCACGCC |
| | UUGGGCGU | | UCUGGCA | | CAAGAUCUG |
| | GACG | | | | GCC |
| 65 | UGCAGUGG | 283 | UGGUGCUCC | 501 | CCUCGUGGU |
| | GAGCACCAC | | CACUGCA | | GCUCCCACU |
| | GAGG | | | | GCA |
| 66 | UUGCAGUGG | 284 | GGUGCUCCC | 502 | CUCGUGGU |
| | GAGCACCAC | | ACUGCAA | | GCUCCCACU |
| | GAG | | | | GCAG |
| 67 | UCCAUCCAG | 285 | UCUACCUCU | 503 | GCUCGUCUA |
| | AGGUAGACG | | GGAUGGA | | CCUCUGGAU |
| | AGC | | | | GGA |
| 68 | UUCCAUCCA | 286 | CUACCUCUG | 504 | CUCGUCUAC |
| | GAGGUAGAC | | GAUGGAA | | CUCUGGAUG |
| | GAG | | | | GAG |
| 69 | UCUCCAUCC | 287 | UACCUCUGG | 505 | UCGUCUACC |
| | AGAGGUAGA | | AUGGAGA | | UCUGGAUGG |
| | CGA | | | | AGC |
| 70 | UGCUCCAUC | 288 | ACCUCUGGA | 506 | CGUCUACCU |
| | CAGAGGUAG | | UGGAGCA | | CUGGAUGGA |
| | ACG | | | | GCG |
| 71 | UUCAGGAAG | 289 | UCAUGGACU | 507 | CCUUCUCAU |
| | UCCAUGAGA | | UCCUGAA | | GGACUUCCU |
| | AGG | | | | GAA |
| 72 | UUUCAGGAA | 290 | CAUGGACUU | 508 | CUUCUCAUG |
| | GUCCAUGAG | | CCUGAAA | | GACUUCCUG |
| | AAG | | | | AAC |
| 73 | UACAGGAUG | 291 | ACGUGGUCA | 509 | CUAUUACGU |
| | ACCACGUAA | | UCCUGUA | | GGUCAUCCU |
| | UAG | | | | GUG |
| 74 | UGACCCGCU | 292 | UGGUCCCAG | 510 | CUCUGUGGU |
| | GGGACCACA | | CGGGUCA | | CCCAGCGGG |
| | GAG | | | | UCA |
| 75 | UUGACCCGC | 293 | GGUCCCAGC | 511 | UCUGUGGUC |
| | UGGGACCAC | | GGGUCAA | | CCAGCGGGU |
| | AGA | | | | CAU |
| 76 | UAUGACCCG | 294 | GUCCCAGCG | 512 | CUGUGGUCC |
| | CUGGGACCA | | GGUCAUA | | CAGCGGGUC |
| | CAG | | | | AUC |
| 77 | UGAUGACCC | 295 | UCCCAGCGG | 513 | UGUGGUCCC |
| | GCUGGGACC | | GUCAUCA | | AGCGGGUCA |
| | ACA | | | | UCU |
| 78 | UAGAUGACC | 296 | CCCAGCGGG | 514 | GUGGUCCCA |
| | CGCUGGGAC | | UCAUCUA | | GCGGGUCAU |
| | CAC | | | | CUA |
| 79 | UUAGAUGAC | 297 | CCAGCGGGU | 515 | UGGUCCCAG |
| | CCGCUGGGA | | CAUCUAA | | CGGGUCAUC |
| | CCA | | | | UAC |
| 80 | UGUAGAUGA | 298 | CAGCGGGUC | 516 | GGUCCCAGC |
| | CCCGCUGG | | AUCUACA | | GGGUCAUCU |
| | GACC | | | | ACC |
| 81 | UGGUAGAUG | 299 | AGCGGGUCA | 517 | GUCCCAGCG |
| | ACCCGCUGG | | UCUACCA | | GGUCAUCUA |
| | GAC | | | | CCU |
| 82 | UAGGUAGAU | 300 | GCGGGUCAU | 518 | UCCCAGCGG |
| | GACCCGCUG | | CUACCUA | | GUCAUCUAC |
| | GGA | | | | CUC |
| 83 | UGAGGUAGA | 301 | CGGGUCAUC | 519 | CCCAGCGGG |
| | UGACCCGCU | | UACCUCA | | UCAUCUACC |
| | GGG | | | | UCC |
| 84 | UGGAGGUAG | 302 | GGGUCAUCU | 520 | CCAGCGGGU |
| | AUGACCCGC | | ACCUCCA | | CAUCUACCU |
| | UGG | | | | CCA |
| 85 | UUGGAGGUA | 303 | GGUCAUCUA | 521 | CAGCGGGUC |
| | GAUGACCCG | | CCUCCAA | | AUCUACCUC |
| | CUG | | | | CAG |
| 86 | UCUGGAGGU | 304 | GUCAUCUAC | 522 | AGCGGGUCA |
| | AGAUGACCC | | CUCCAGA | | UCUACCUCC |
| | GCU | | | | AGG |
| 87 | UCCUGGAGG | 305 | UCAUCUACC | 523 | GCGGGUCAU |
| | UAGAUGACC | | UCCAGGA | | CUACCUCCA |
| | CGC | | | | GGG |
| 88 | UGCCCUGGA | 306 | AUCUACCUC | 524 | GGGUCAUCU |
| | GGUAGAUGA | | CAGGGCA | | ACCUCCAGG |
| | CCC | | | | GCU |
| 89 | UAGCCCUGG | 307 | UCUACCUCC | 525 | GGUCAUCUA |
| | AGGUAGAUG | | AGGGCUA | | CCUCCAGGG |
| | ACC | | | | CUC |
| 90 | UGAGCCCUG | 308 | CUACCUCCA | 526 | GUCAUCUAC |
| | GAGGUAGAU | | GGGCUCA | | CUCCAGGGC |
| | GAC | | | | UCU |
| 91 | UAGAGCCCU | 309 | UACCUCCAG | 527 | UCAUCUACC |
| | GGAGGUAGA | | GGCUCUA | | UCCAGGGCU |
| | UGA | | | | CUG |
| 92 | UCAGAGCCC | 310 | ACCUCCAGG | 528 | CAUCUACCU |
| | UGGAGGUAG | | GCUCUGA | | CCAGGGCUC |
| | AUG | | | | UGC |
| 93 | UGCAGAGCC | 311 | CCUCCAGGG | 529 | AUCUACCUC |
| | CUGGAGGUA | | CUCUGCA | | CAGGGCUCU |
| | GAU | | | | GCA |
| 94 | UUCUUGGCU | 312 | UCAUGCGAG | 530 | GGACCUCAU |
| | CGCAUGAGG | | CCAAGAA | | GCGAGCCAA |
| | UCC | | | | GAU |
| 95 | UAUCUUGGC | 313 | CAUGCGAGC | 531 | GACCUCAUG |
| | UCGCAUGAG | | CAAGAUA | | CGAGCCAAG |
| | GUC | | | | AUG |
| 96 | UCAUCUUGG | 314 | AUGCGAGCC | 532 | ACCUCAUGC |
| | CUCGCAUGA | | AAGAUGA | | GAGCCAAGA |
| | GGU | | | | UGG |
| 97 | UCCAUCUUG | 315 | UGCGAGCCA | 533 | CCUCAUGCG |
| | GCUCGCAUG | | AGAUGGA | | AGCCAAGAU |
| | AGG | | | | GGA |
| 98 | UUCCAUCUU | 316 | GCGAGCCAA | 534 | CUCAUGCGA |
| | GGCUCGCAU | | GAUGGAA | | GCCAAGAUG |
| | GAG | | | | GAC |
| 99 | UGUCCAUCU | 317 | CGAGCCAAG | 535 | UCAUGCGAG |
| | UGGCUCGCA | | AUGGACA | | CCAAGAUGG |
| | UGA | | | | ACA |
| 100 | UUGUCCAUC | 318 | GAGCCAAGA | 536 | CAUGCGAGC |
| | UUGGCUCGC | | UGGACAA | | CAAGAUGGA |
| | AUG | | | | CAA |
| 101 | UUUGUCCAU | 319 | AGCCAAGAU | 537 | AUGCGAGCC |
| | CUUGGCUCG | | GGACAAA | | AAGAUGGAC |
| | CAU | | | | AAU |
| 102 | UCCACGUGG | 320 | UUGCUGACC | 538 | CAAGUUUGC |
| | UCAGCAAAC | | ACGUGGA | | UGACCACGU |
| | UUG | | | | GGU |
| 103 | UGGCAGAUG | 321 | UGAACUGCA | 539 | GGCGCUGAA |
| | CAGUUCAGC | | UCUGCCA | | CUGCAUCUG |
| | GCC | | | | CCC |
| 104 | UGGGCAGAU | 322 | GAACUGCAU | 540 | GCGCUGAAC |
| | GCAGUUCAG | | CUGCCCA | | UGCAUCUGC |
| | CGC | | | | CCG |
| 105 | UCGGGCAGA | 323 | AACUGCAUC | 541 | CGCUGAACU |
| | UGCAGUUCA | | UGCCCGA | | GCAUCUGCC |
| | GCG | | | | CGG |
| 106 | UCCGGGCAG | 324 | ACUGCAUCU | 542 | GCUGAACUG |
| | AUGCAGUUC | | GCCCGGA | | CAUCUGCCC |
| | AGC | | | | GGC |
| 107 | UGCCGGGCA | 325 | CUGCAUCUG | 543 | CUGAACUGC |
| | GAUGCAGUU | | CCCGGCA | | AUCUGCCCG |
| | CAG | | | | GCG |
| 108 | UAGGGUGAU | 326 | CACCCUCAU | 544 | ACCUCCACC |
| | GAGGGUGG | | CACCCUA | | CUCAUCACC |
| | AGGU | | | | CUG |
| 109 | UCAGGGUGA | 327 | ACCCUCAUC | 545 | CCUCCACCC |
| | UGAGGGUG | | ACCCUGA | | UCAUCACCC |
| | GAGG | | | | UGC |
| 110 | UGCAGGGU | 328 | CCCUCAUCA | 546 | CUCCACCCU |
| | GAUGAGGGU | | CCCUGCA | | CAUCACCCU |
| | GGAG | | | | GCU |
| 111 | UGCACCAGC | 329 | UCACCCUGC | 547 | CCUCAUCAC |
| | AGGGUGAUG | | UGGUGCA | | CCUGCUGGU |
| | AGG | | | | GCA |
| 112 | UGACGUGUG | 330 | GCUGGUGCA | 548 | ACCCUGCUG |
| | CACCAGCAG | | CACGUCA | | GUGCACACG |
| | GGU | | | | UCC |
| 113 | UUGCGCUGC | 331 | AGCAGUGGC | 549 | UCCGGAGCA |
| | CACUGCUCC | | AGCGCAA | | GUGGCAGC |
| | GGA | | | | GCAU |
| 114 | UUGAAGCUC | 332 | AGGGCAAGA | 550 | GUACGAGGG |
| | UUGCCCUCG | | GCUUCAA | | CAAGAGCUU |
| | UAC | | | | CAC |
| 115 | UUAGGUGAA | 333 | CAAGAGCUU | 551 | GAGGGCAAG |
| | GCUCUUGCC | | CACCUAA | | AGCUUCACC |
| | CUC | | | | UAC |
| 116 | UGUAGGUGA | 334 | AAGAGCUUC | 552 | AGGGCAAGA |
| | AGCUCUUGC | | ACCUACA | | GCUUCACCU |
| | CCU | | | | ACG |
| 117 | UCGUAGGUG | 335 | AGAGCUUCA | 553 | GGGCAAGAG |
| | AAGCUCUUG | | CCUACGA | | CUUCACCUA |
| | CCC | | | | CGC |
| 118 | UGCGUAGGU | 336 | GAGCUUCAC | 554 | GGCAAGAGC |
| | GAAGCUCUU | | CUACGCA | | UUCACCUAC |
| | GCC | | | | GCG |
| 119 | UGCCCGAUG | 337 | UGUGCCUCA | 555 | UGGCGUGU |
| | AGGCACACG | | UCGGGCA | | GCCUCAUCG |
| | CCA | | | | GGCU |
| 120 | UCAGCCCGA | 338 | UGCCUCAUC | 556 | GCGUGUGC |
| | UGAGGCACA | | GGGCUGA | | CUCAUCGGG |
| | CGC | | | | CUGA |
| 121 | UUCAGCCCG | 339 | GCCUCAUCG | 557 | CGUGUGCCU |
| | AUGAGGCAC | | GGCUGAA | | CAUCGGGCU |
| | ACG | | | | GAA |
| 122 | UUUCAGCCC | 340 | CCUCAUCGG | 558 | GUGUGCCUC |
| | GAUGAGGCA | | GCUGAAA | | AUCGGGCUG |
| | CAC | | | | AAG |
| 123 | UGCUUCAGC | 341 | UCAUCGGGC | 559 | GUGCCUCAU |
| | CCGAUGAGG | | UGAAGCA | | CGGGCUGAA |
| | CAC | | | | GCG |
| 124 | UCGCUUCAG | 342 | CAUCGGGCU | 560 | UGCCUCAUC |
| | CCCGAUGAG | | GAAGCGA | | GGGCUGAAG |
| | GCA | | | | CGG |
| 125 | UAGUUCUCC | 343 | CCAAGGAGG | 561 | CAUCACCAA |
| | UCCUUGGUG | | AGAACUA | | GGAGGAGAA |
| | AUG | | | | CUC |
| 126 | UGAGUUCUC | 344 | CAAGGAGGA | 562 | AUCACCAAG |
| | CUCCUUGGU | | GAACUCA | | GAGGAGAAC |
| | GAU | | | | UCG |
| 127 | UACAGGCAG | 345 | CCACCUCCU | 563 | CUGUGCCAC |
| | GAGGUGGCA | | GCCUGUA | | CUCCUGCCU |
| | CAG | | | | GUG |
| 128 | UCACAGGCA | 346 | CACCUCCUG | 564 | UGUGCCACC |
| | GGAGGUGG | | CCUGUGA | | UCCUGCCUG |
| | CACA | | | | UGA |
| 129 | UUCACAGGC | 347 | ACCUCCUGC | 565 | GUGCCACCU |
| | AGGAGGUG | | CUGUGAA | | CCUGCCUGU |
| | GCAC | | | | GAA |
| 130 | UUUCACAGG | 348 | CCUCCUGCC | 566 | UGCCACCUC |
| | CAGGAGGUG | | UGUGAAA | | CUGCCUGUG |
| | GCA | | | | AAA |
| 131 | UUUGCAGCC | 349 | GGACAAGGG | 567 | CGGCUGGAC |
| | CUUGUCCAG | | CUGCAAA | | AAGGGCUGC |
| | CCG | | | | AAG |
| 132 | UGCUUGUUC | 350 | GGUUCAAGA | 568 | CUACGGGUU |
| | UUGAACCCG | | ACAAGCA | | CAAGAACAA |
| | UAG | | | | GCU |
| 133 | UAGCUUGUU | 351 | GUUCAAGAA | 569 | UACGGGUUC |
| | CUUGAACCC | | CAAGCUA | | AAGAACAAG |
| | GUA | | | | CUG |
| 134 | UCAGCUUGU | 352 | UUCAAGAAC | 570 | ACGGGUUCA |
| | UCUUGAACC | | AAGCUGA | | AGAACAAGC |
| | CGU | | | | UGA |
| 135 | UUCAGCUUG | 353 | UCAAGAACA | 571 | CGGGUUCAA |
| | UUCUUGAAC | | AGCUGAA | | GAACAAGCU |
| | CCG | | | | GAU |
| 136 | UAUCAGCUU | 354 | CAAGAACAA | 572 | GGGUUCAAG |
| | GUUCUUGAA | | GCUGAUA | | AACAAGCUG |
| | CCC | | | | AUC |
| 137 | UGCACGAUG | 355 | ACAACUUCA | 573 | GCUGUACAA |
| | AAGUUGUAC | | UCGUGCA | | CUUCAUCGU |
| | AGC | | | | GCC |
| 138 | UCCAGGAAG | 356 | ACCACCACU | 574 | GCCCGACCA |
| | UGGUGGUC | | UCCUGGA | | CCACUUCCU |
| | GGGC | | | | GGA |
| 139 | UUCCAGGAA | 357 | CCACCACUU | 575 | CCCGACCAC |
| | GUGGUGGU | | CCUGGAA | | CACUUCCUG |
| | CGGG | | | | GAA |
| 140 | UUCCAUGUA | 358 | GGUCUACUA | 576 | CCCAUGGUC |
| | GUAGACCAU | | CAUGGAA | | UACUACAUG |
| | GGG | | | | GAG |
| 141 | UCUCCAUGU | 359 | GUCUACUAC | 577 | CCAUGGUCU |
| | AGUAGACCA | | AUGGAGA | | ACUACAUGG |
| | UGG | | | | AGG |
| 142 | UCCUCCAUG | 360 | UCUACUACA | 578 | CAUGGUCUA |
| | UAGUAGACC | | UGGAGGA | | CUACAUGGA |
| | AUG | | | | GGG |
| 143 | UUGUCCAGG | 361 | UGGACAACC | 579 | CUCUGUGGA |
| | UUGUCCACA | | UGGACAA | | CAACCUGGA |
| | GAG | | | | CAG |
| 144 | UGCUGUCCA | 362 | GACAACCUG | 580 | CUGUGGACA |
| | GGUUGUCCA | | GACAGCA | | ACCUGGACA |
| | CAG | | | | GCC |
| 145 | UCCUUGUCC | 363 | UGGUGGUG | 581 | CCUGGUGG |
| | ACCACCACC | | GACAAGGA | | UGGUGGACA |
| | AGG | | | | AGGA |
| 146 | UUCCUUGUC | 364 | GGUGGUGG | 582 | CUGGUGGU |
| | CACCACCAC | | ACAAGGAA | | GGUGGACAA |
| | CAG | | | | GGAG |
| 147 | UCUCCUUGU | 365 | GUGGUGGA | 583 | UGGUGGUG |
| | CCACCACCA | | CAAGGAGA | | GUGGACAAG |
| | CCA | | | | GAGA |
| 148 | UGCUCUCCU | 366 | GUGGACAAG | 584 | UGGUGGUG |
| | UGUCCACCA | | GAGAGCA | | GACAAGGAG |
| | CCA | | | | AGCA |
| 149 | UUGCUCUCC | 367 | UGGACAAGG | 585 | GGUGGUGG |
| | UUGUCCACC | | AGAGCAA | | ACAAGGAGA |
| | ACC | | | | GCAC |
| 150 | UGUGCUCUC | 368 | GGACAAGGA | 586 | GUGGUGGA |
| | CUUGUCCAC | | GAGCACA | | CAAGGAGAG |
| | CAC | | | | CACC |
| 151 | UUGACGAUG | 369 | CCAAGACCA | 587 | GGACGCCAA |
| | GUCUUGGC | | UCGUCAA | | GACCAUCGU |
| | GUCC | | | | CAA |
| 152 | UUUGACGAU | 370 | CAAGACCAU | 588 | GACGCCAAG |
| | GGUCUUGG | | CGUCAAA | | ACCAUCGUC |
| | CGUC | | | | AAC |
| 153 | UCCGUGGU | 371 | UCAGCAUCA | 589 | CAGCCUCAG |
| | GAUGCUGAG | | CCACGGA | | CAUCACCAC |
| | GCUG | | | | GGA |
| 154 | UCUCCGUGG | 372 | AGCAUCACC | 590 | GCCUCAGCA |
| | UGAUGCUGA | | ACGGAGA | | UCACCACGG |
| | GGC | | | | AGC |
| 155 | UGCUCCGUG | 373 | GCAUCACCA | 591 | CCUCAGCAU |
| | GUGAUGCUG | | CGGAGCA | | CACCACGGA |
| | AGG | | | | GCU |
| 156 | UGAGCUCCG | 374 | AUCACCACG | 592 | UCAGCAUCA |
| | UGGUGAUGC | | GAGCUCA | | CCACGGAGC |
| | UGA | | | | UCA |
| 157 | UUGAGCUCC | 375 | UCACCACGG | 593 | CAGCAUCAC |
| | GUGGUGAU | | AGCUCAA | | CACGGAGCU |
| | GCUG | | | | CAC |
| 158 | UGUGAGCUC | 376 | CACCACGGA | 594 | AGCAUCACC |
| | CGUGGUGAU | | GCUCACA | | ACGGAGCUC |
| | GCU | | | | ACC |
| 159 | UGCAUGUUG | 377 | ACCCUUCCA | 595 | CACCCACCC |
| | GAAGGGUG | | ACAUGCA | | UUCCAACAU |
| | GGUG | | | | GCG |
| 160 | UCGCAUGUU | 378 | CCCUUCCAA | 596 | ACCCACCCU |
| | GGAAGGGU | | CAUGCGA | | UCCAACAUG |
| | GGGU | | | | CGC |
| 161 | UAGUAGCUG | 379 | CCAAGGACA | 597 | CCGCGCCAA |
| | UCCUUGGCG | | GCUACUA | | GGACAGCUA |
| | CGG | | | | CUC |
| 162 | UGAGUAGCU | 380 | CAAGGACAG | 598 | CGCGCCAAG |
| | GUCCUUGGC | | CUACUCA | | GACAGCUAC |
| | GCG | | | | UCU |
| 163 | UAGAGUAGC | 381 | AAGGACAGC | 599 | GCGCCAAGG |
| | UGUCCUUGG | | UACUCUA | | ACAGCUACU |
| | CGC | | | | CUC |
| 164 | UGAGAGUAG | 382 | AGGACAGCU | 600 | CGCCAAGGA |
| | CUGUCCUUG | | ACUCUCA | | CAGCUACUC |
| | GCG | | | | UCU |
| 165 | UAGAGAGUA | 383 | GGACAGCUA | 601 | GCCAAGGAC |
| | GCUGUCCUU | | CUCUCUA | | AGCUACUCU |
| | GGC | | | | CUG |
| 166 | UCAGAGAGU | 384 | GACAGCUAC | 602 | CCAAGGACA |
| | AGCUGUCCU | | UCUCUGA | | GCUACUCUC |
| | UGG | | | | UGG |
| 167 | UCCAGAGAG | 385 | ACAGCUACU | 603 | CAAGGACAG |
| | UAGCUGUCC | | CUCUGGA | | CUACUCUCU |
| | UUG | | | | GGC |
| 168 | UGCCAGAGA | 386 | CAGCUACUC | 604 | AAGGACAGC |
| | GUAGCUGUC | | UCUGGCA | | UACUCUCUG |
| | CUU | | | | GCU |
| 169 | UAGCCAGAG | 387 | AGCUACUCU | 605 | AGGACAGCU |
| | AGUAGCUGU | | CUGGCUA | | ACUCUCUGG |
| | CCU | | | | CUC |
| 170 | UGAAAGAGC | 388 | CUCUCUGGC | 606 | AGCUACUCU |
| | CAGAGAGUA | | UCUUUCA | | CUGGCUCUU |
| | GCU | | | | UCC |
| 171 | UGGAAAGAG | 389 | UCUCUGGCU | 607 | GCUACUCUC |
| | CCAGAGAGU | | CUUUCCA | | UGGCUCUUU |
| | AGC | | | | CCA |
| 172 | UUGGAAAGA | 390 | CUCUGGCUC | 608 | CUACUCUCU |
| | GCCAGAGAG | | UUUCCAA | | GGCUCUUUC |
| | UAG | | | | CAA |
| 173 | UUUGGAAAG | 391 | UCUGGCUCU | 609 | UACUCUCUG |
| | AGCCAGAGA | | UUCCAAA | | GCUCUUUCC |
| | GUA | | | | AAA |
| 174 | UUUUGGAAA | 392 | CUGGCUCUU | 610 | ACUCUCUGG |
| | GAGCCAGAG | | UCCAAAA | | CUCUUUCCA |
| | AGU | | | | AAC |
| 175 | UGUUUGGAA | 393 | UGGCUCUUU | 611 | CUCUCUGGC |
| | AGAGCCAGA | | CCAAACA | | UCUUUCCAA |
| | GAG | | | | ACU |
| 176 | UAGUUUGGA | 394 | GGCUCUUUC | 612 | UCUCUGGCU |
| | AAGAGCCAG | | CAAACUA | | CUUUCCAAA |
| | AGA | | | | CUA |
| 177 | UAGGCCAGG | 395 | GCUCCAACC | 613 | GAAUGGCUC |
| | UUGGAGCCA | | UGGCCUA | | CAACCUGGC |
| | UUC | | | | CUU |
| 178 | UGAAGGCCA | 396 | UCCAACCUG | 614 | AUGGCUCCA |
| | GGUUGGAG | | GCCUUCA | | ACCUGGCCU |
| | CCAU | | | | UCA |
| 179 | UACAUGAAG | 397 | ACCUGGCCU | 615 | CUCCAACCU |
| | GCCAGGUUG | | UCAUGUA | | GGCCUUCAU |
| | GAG | | | | GUU |
| 180 | UAACAUGAA | 398 | CCUGGCCUU | 616 | UCCAACCUG |
| | GGCCAGGUU | | CAUGUUA | | GCCUUCAUG |
| | GGA | | | | UUC |
| 181 | UGAACAUGA | 399 | CUGGCCUUC | 617 | CCAACCUGG |
| | AGGCCAGGU | | AUGUUCA | | CCUUCAUGU |
| | UGG | | | | UCC |
| 182 | UGGAACAUG | 400 | UGGCCUUCA | 618 | CAACCUGGC |
| | AAGGCCAGG | | UGUUCCA | | CUUCAUGUU |
| | UUG | | | | CCG |
| 183 | UGCGGAACA | 401 | GCCUUCAUG | 619 | ACCUGGCCU |
| | UGAAGGCCA | | UUCCGCA | | UCAUGUUCC |
| | GGU | | | | GCC |
| 184 | UGCAGGCG | 402 | UCAUGUUCC | 620 | GGCCUUCAU |
| | GAACAUGAA | | GCCUGCA | | GUUCCGCCU |
| | GGCC | | | | GCC |
| 185 | UGGCAGGC | 403 | CAUGUUCCG | 621 | GCCUUCAUG |
| | GGAACAUGA | | CCUGCCA | | UUCCGCCUG |
| | AGGC | | | | CCG |
| 186 | UGAAGGACU | 404 | CUCUACCAG | 622 | CACUGCUCU |
| | GGUAGAGCA | | UCCUUCA | | ACCAGUCCU |
| | GUG | | | | UCG |
| 187 | UCGAAGGAC | 405 | UCUACCAGU | 623 | ACUGCUCUA |
| | UGGUAGAGC | | CCUUCGA | | CCAGUCCUU |
| | AGU | | | | CGU |
| 188 | UACGAAGGA | 406 | CUACCAGUC | 624 | CUGCUCUAC |
| | CUGGUAGAG | | CUUCGUA | | CAGUCCUUC |
| | CAG | | | | GUG |
| 189 | UCACGAAGG | 407 | UACCAGUCC | 625 | UGCUCUACC |
| | ACUGGUAGA | | UUCGUGA | | AGUCCUUCG |
| | GCA | | | | UGA |
| 190 | UUCACGAAG | 408 | ACCAGUCCU | 626 | GCUCUACCA |
| | GACUGGUAG | | UCGUGAA | | GUCCUUCGU |
| | AGC | | | | GAA |
| 191 | UUUCACGAA | 409 | CCAGUCCUU | 627 | CUCUACCAG |
| | GGACUGGUA | | CGUGAAA | | UCCUUCGUG |
| | GAG | | | | AAG |
| 192 | UCUUCACGA | 410 | CAGUCCUUC | 628 | UCUACCAGU |
| | AGGACUGGU | | GUGAAGA | | CCUUCGUGA |
| | AGA | | | | AGG |
| 193 | UCCUUCACG | 41 | AGUCCUUCG | 629 | CUACCAGUC |
| | AAGGACUGG | | UGAAGGA | | CUUCGUGAA |
| | UAG | | | | GGA |
| 194 | UAUGUAGUC | 412 | CGUGAAGGA | 630 | UCCUUCGUG |
| | CUUCACGAA | | CUACAUA | | AAGGACUAC |
| | GGA | | | | AUG |
| 195 | UCAUGUAGU | 413 | GUGAAGGAC | 631 | CCUUCGUGA |
| | CCUUCACGA | | UACAUGA | | AGGACUACA |
| | AGG | | | | UGA |
| 196 | UUCAUGUAG | 414 | UGAAGGACU | 632 | CUUCGUGAA |
| | UCCUUCACG | | ACAUGAA | | GGACUACAU |
| | AAG | | | | GAU |
| 197 | UAUCAUGUA | 415 | GAAGGACUA | 633 | UUCGUGAAG |
| | GUCCUUCAC | | CAUGAUA | | GACUACAUG |
| | GAA | | | | AUC |
| 198 | UGAUCAUGU | 416 | AAGGACUAC | 634 | UCGUGAAGG |
| | AGUCCUUCA | | AUGAUCA | | ACUACAUGA |
| | CGA | | | | UCA |
| 199 | UUGAUCAUG | 417 | AGGACUACA | 635 | CGUGAAGGA |
| | UAGUCCUUC | | UGAUCAA | | CUACAUGAU |
| | ACG | | | | CAC |
| 200 | UGUGAUCAU | 418 | GGACUACAU | 636 | GUGAAGGAC |
| | GUAGUCCUU | | GAUCACA | | UACAUGAUC |
| | CAC | | | | ACC |
| 201 | UGGUGAUCA | 419 | GACUACAUG | 637 | UGAAGGACU |
| | UGUAGUCCU | | AUCACCA | | ACAUGAUCA |
| | UCA | | | | CCA |
| 202 | UUGGUGAUC | 420 | ACUACAUGA | 638 | GAAGGACUA |
| | AUGUAGUCC | | UCACCAA | | CAUGAUCAC |
| | UUC | | | | CAU |
| 203 | UGAUGGUGA | 421 | UACAUGAUC | 639 | AGGACUACA |
| | UCAUGUAGU | | ACCAUCA | | UGAUCACCA |
| | CCU | | | | UCA |
| 204 | UUGAUGGUG | 422 | ACAUGAUCA | 640 | GGACUACAU |
| | AUCAUGUAG | | CCAUCAA | | GAUCACCAU |
| | UCC | | | | CAC |
| 205 | UGUGAUGGU | 423 | CAUGAUCAC | 641 | GACUACAUG |
| | GAUCAUGUA | | CAUCACA | | AUCACCAUC |
| | GUC | | | | ACC |
| 206 | UGGUGAUG | 424 | AUGAUCACC | 642 | ACUACAUGA |
| | GUGAUCAUG | | AUCACCA | | UCACCAUCA |
| | UAGU | | | | CCC |
| 207 | UGCGGAUCC | 425 | GACCUGUGG | 643 | AGGGCGACC |
| | ACAGGUCGC | | AUCCGCA | | UGUGGAUCC |
| | CCU | | | | GCA |
| 208 | UGGAAGAGG | 426 | ACGGCCGCC | 644 | CACGUACGG |
| | CGGCCGUAC | | UCUUCCA | | CCGCCUCUU |
| | GUG | | | | CCA |
| 209 | UCUGGAAGA | 427 | GGCCGCCUC | 645 | CGUACGGCC |
| | GGCGGCCG | | UUCCAGA | | GCCUCUUCC |
| | UACG | | | | AGA |
| 210 | UUCUGGAAG | 428 | GCCGCCUCU | 646 | GUACGGCCG |
| | AGGCGGCC | | UCCAGAA | | CCUCUUCCA |
| | GUAC | | | | GAA |
| 211 | UUUCUGGAA | 429 | CCGCCUCUU | 647 | UACGGCCGC |
| | GAGGCGGC | | CCAGAAA | | CUCUUCCAG |
| | CGUA | | | | AAG |
| 212 | UGGCGCUG | 430 | UGCUCCUCC | 648 | AGCUCUGCU |
| | GAGGAGCAG | | AGCGCCA | | CCUCCAGCG |
| | AGCU | | | | CCG |
| 213 | UCUCGGCGC | 431 | UCCUCCAGC | 649 | UCUGCUCCU |
| | UGGAGGAGC | | GCCGAGA | | CCAGCGCCG |
| | AGA | | | | AGA |
| 214 | UAUCUCGGC | 432 | CUCCAGCGC | 650 | UGCUCCUCC |
| | GCUGGAGGA | | CGAGAUA | | AGCGCCGAG |
| | GCA | | | | AUC |
| 215 | UGAUCUCGG | 433 | UCCAGCGCC | 651 | GCUCCUCCA |
| | CGCUGGAG | | GAGAUCA | | GCGCCGAGA |
| | GAGC | | | | UCC |
| 216 | UGGAUCUCG | 434 | CCAGCGCCG | 652 | CUCCUCCAG |
| | GCGCUGGA | | AGAUCCA | | CGCCGAGAU |
| | GGAG | | | | CCC |
| 217 | UGGGAUCUC | 435 | CAGCGCCGA | 653 | UCCUCCAGC |
| | GGCGCUGG | | GAUCCCA | | GCCGAGAUC |
| | AGGA | | | | CCC |
| 218 | UUGGGAGCU | 436 | GGCCAGCAG | 654 | CACGUGGCC |
| | GCUGGCCAC | | CUCCCAA | | AGCAGCUCC |
| | GUG | | | | CAG |
| 655 | UGGCGGUG | 845 | CUCCUGGAC | 1035 | GCGCGCUCC |
| | UCCAGGAGC | | ACCGCCA | | UGGACACCG |
| | GCGC | | | | CCG |
| 656 | UGCAAGGGC | 846 | AGGUGCUGC | 1036 | CUCCGAGGU |
| | AGCACCUCG | | CCUUGCA | | GCUGCCCUU |
| | GAG | | | | GCC |
| 657 | UGUCGUUCU | 847 | UCCUUCCAG | 1037 | ACCCGUCCU |
| | GGAAGGACG | | AACGACA | | UCCAGAACG |
| | GGU | | | | ACG |
| 658 | UGAACUCCA | 848 | GUCCAGGUG | 1038 | ACAGGGUCC |
| | CCUGGACCC | | GAGUUCA | | AGGUGGAGU |
| | UGU | | | | UCU |
| 659 | UAGAACUCC | 849 | UCCAGGUGG | 1039 | CAGGGUCCA |
| | ACCUGGACC | | AGUUCUA | | GGUGGAGU |
| | CUG | | | | UCUA |
| 660 | UUAGAACUC | 850 | CCAGGUGGA | 1040 | AGGGUCCAG |
| | CACCUGGAC | | GUUCUAA | | GUGGAGUUC |
| | CCU | | | | UAC |
| 661 | UUGAGCCGC | 851 | UCAAGGAGC | 1041 | CACCUUCAA |
| | UCCUUGAAG | | GGCUCAA | | GGAGCGGC |
| | GUG | | | | UCAA |
| 662 | UCAGCCGGA | 852 | CUGAGGAUC | 1042 | CGAGCCUGA |
| | UCCUCAGGC | | CGGCUGA | | GGAUCCGGC |
| | UCG | | | | UGU |
| 663 | UACAGCCGG | 853 | UGAGGAUCC | 1043 | GAGCCUGAG |
| | AUCCUCAGG | | GGCUGUA | | GAUCCGGCU |
| | CUC | | | | GUU |
| 664 | UAACAGCCG | 854 | GAGGAUCCG | 1044 | AGCCUGAGG |
| | GAUCCUCAG | | GCUGUUA | | AUCCGGCUG |
| | GCU | | | | UUC |
| 665 | UGAACAGCC | 855 | AGGAUCCGG | 1045 | GCCUGAGGA |
| | GGAUCCUCA | | CUGUUCA | | UCCGGCUGU |
| | GGC | | | | UCA |
| 666 | UCAGGGAGA | 856 | UUCAACUUC | 1046 | GGCUGUUCA |
| | AGUUGAACA | | UCCCUGA | | ACUUCUCCC |
| | GCC | | | | UGA |
| 667 | UUUCAGGGA | 857 | CAACUUCUC | 1047 | CUGUUCAAC |
| | GAAGUUGAA | | CCUGAAA | | UUCUCCCUG |
| | CAG | | | | AAG |
| 668 | UCUUCAGGG | 858 | AACUUCUCC | 1048 | UGUUCAACU |
| | AGAAGUUGA | | CUGAAGA | | UCUCCCUGA |
| | ACA | | | | AGC |
| 669 | UGUAGUUCU | 859 | CCAAAGCAG | 1049 | GCUGCCCAA |
| | GCUUUGGG | | AACUACA | | AGCAGAACU |
| | CAGC | | | | ACU |
| 670 | UGAGUAGUU | 860 | AAAGCAGAA | 1050 | UGCCCAAAG |
| | CUGCUUUGG | | CUACUCA | | CAGAACUAC |
| | GCA | | | | UCC |
| 671 | UACGAGUCA | 861 | CCUUCAAUG | 1051 | CUACUCCUU |
| | UUGAAGGAG | | ACUCGUA | | CAAUGACUC |
| | UAG | | | | GUC |
| 672 | UGAGGACGA | 862 | CAAUGACUC | 1052 | UCCUUCAAU |
| | GUCAUUGAA | | GUCCUCA | | GACUCGUCC |
| | GGA | | | | UCC |
| 673 | UCCAGUUGA | 863 | UCCGAGAUC | 1053 | CGUCCUCCG |
| | UCUCGGAGG | | AACUGGA | | AGAUCAACU |
| | ACG | | | | GGG |
| 674 | UCCCAGUUG | 864 | CCGAGAUCA | 1054 | GUCCUCCGA |
| | AUCUCGGAG | | ACUGGGA | | GAUCAACUG |
| | GAC | | | | GGC |
| 675 | UGAGCCCAG | 865 | AGAUCAACU | 1055 | CUCCGAGAU |
| | UUGAUCUCG | | GGGCUCA | | CAACUGGGC |
| | GAG | | | | UCC |
| 676 | UGUCAUCUU | 866 | GGAGAGAAA | 1056 | UGGGUGGA |
| | UCUCUCCAC | | GAUGACA | | GAGAAAGAU |
| | CCA | | | | GACA |
| 677 | UCAGUGUCA | 867 | AGAAAGAUG | 1057 | UGGAGAGAA |
| | UCUUUCUCU | | ACACUGA | | AGAUGACAC |
| | CCA | | | | UGU |
| 678 | UCACAGUGU | 868 | AAAGAUGAC | 1058 | GAGAGAAAG |
| | CAUCUUUCU | | ACUGUGA | | AUGACACUG |
| | CUC | | | | UGG |
| 679 | UGCCACGAU | 869 | CCAGGUCAU | 1059 | GCGAUCCAG |
| | GACCUGGAU | | CGUGGCA | | GUCAUCGUG |
| | CGC | | | | GCC |
| 680 | UGAAGCUUA | 870 | GCCAUAAUA | 1060 | UCGUGGCCA |
| | UUAUGGCCA | | AGCUUCA | | UAAUAAGCU |
| | CGA | | | | UCC |
| 681 | UGGAAGCUU | 871 | CCAUAAUAA | 1061 | CGUGGCCAU |
| | AUUAUGGCC | | GCUUCCA | | AAUAAGCUU |
| | ACG | | | | CCU |
| 682 | UCAGGAAGC | 872 | AUAAUAAGC | 1062 | UGGCCAUAA |
| | UUAUUAUGG | | UUCCUGA | | UAAGCUUCC |
| | CCA | | | | UGG |
| 683 | UGAGGUAGA | 873 | CUUCUCAUC | 1063 | CGAUGCUUC |
| | UGAGAAGCA | | UACCUCA | | UCAUCUACC |
| | UCG | | | | UCA |
| 684 | UUGCUCCCA | 874 | CAACAUCUG | 1064 | AAAGGCAAC |
| | GAUGUUGCC | | GGAGCAA | | AUCUGGGAG |
| | UUU | | | | CAG |
| 685 | UCUGCUCCC | 875 | AACAUCUGG | 1065 | AAGGCAACA |
| | AGAUGUUGC | | GAGCAGA | | UCUGGGAGC |
| | CUU | | | | AGA |
| 686 | UGGACGAAG | 876 | GCGUGUCCU | 1066 | CUUCCGCGU |
| | GACACGCGG | | UCGUCCA | | GUCCUUCGU |
| | AAG | | | | CCU |
| 687 | UCAGGACGA | 877 | GUGUCCUUC | 1067 | UCCGCGUGU |
| | AGGACACGC | | GUCCUGA | | CCUUCGUCC |
| | GGA | | | | UGG |
| 688 | UUGAUCAUC | 878 | UCCUGGAGA | 1068 | CUUCGUCCU |
| | UCCAGGACG | | UGAUCAA | | GGAGAUGAU |
| | AAG | | | | CAA |
| 689 | UUUGAUCAU | 879 | CCUGGAGAU | 1069 | UUCGUCCUG |
| | CUCCAGGAC | | GAUCAAA | | GAGAUGAUC |
| | GAA | | | | AAC |
| 690 | UGUUGAUCA | 880 | CUGGAGAUG | 1070 | UCGUCCUGG |
| | UCUCCAGGA | | AUCAACA | | AGAUGAUCA |
| | CGA | | | | ACA |
| 691 | UUGUUGAUC | 881 | UGGAGAUGA | 1071 | CGUCCUGGA |
| | AUCUCCAGG | | UCAACAA | | GAUGAUCAA |
| | ACG | | | | CAC |
| 692 | UAGUGUUGA | 882 | GAGAUGAUC | 1072 | UCCUGGAGA |
| | UCAUCUCCA | | AACACUA | | UGAUCAACA |
| | GGA | | | | CUC |
| 693 | UGAGUGUUG | 883 | AGAUGAUCA | 1073 | CCUGGAGAU |
| | AUCAUCUCC | | ACACUCA | | GAUCAACAC |
| | AGG | | | | UCU |
| 694 | UAGAGUGUU | 884 | GAUGAUCAA | 1074 | CUGGAGAUG |
| | GAUCAUCUC | | CACUCUA | | AUCAACACU |
| | CAG | | | | CUG |
| 695 | UCAGAGUGU | 885 | AUGAUCAAC | 1075 | UGGAGAUGA |
| | UGAUCAUCU | | ACUCUGA | | UCAACACUC |
| | CCA | | | | UGC |
| 696 | UAUGAUGAA | 886 | UCUGCCCUU | 1076 | AACACUCUG |
| | GGGCAGAGU | | CAUCAUA | | CCCUUCAUC |
| | GUU | | | | AUC |
| 697 | UGAUGAUGA | 887 | CUGCCCUUC | 1077 | ACACUCUGC |
| | AGGGCAGAG | | AUCAUCA | | CCUUCAUCA |
| | UGU | | | | UCA |
| 698 | UUCGUGAUG | 888 | CCUUCAUCA | 1078 | UCUGCCCUU |
| | AUGAAGGGC | | UCACGAA | | CAUCAUCAC |
| | AGA | | | | GAU |
| 699 | UAUCGUGAU | 889 | CUUCAUCAU | 1079 | CUGCCCUUC |
| | GAUGAAGGG | | CACGAUA | | AUCAUCACG |
| | CAG | | | | AUC |
| 700 | UGAUCGUGA | 890 | UUCAUCAUC | 1080 | UGCCCUUCA |
| | UGAUGAAGG | | ACGAUCA | | UCAUCACGA |
| | GCA | | | | UCU |
| 701 | UAGAAGAUC | 891 | UCAUCACGA | 1081 | CUUCAUCAU |
| | GUGAUGAUG | | UCUUCUA | | CACGAUCUU |
| | AAG | | | | CUG |
| 702 | UCAGGUUCC | 892 | CCGCUGCG | 1082 | GGCCGCCG |
| | GCAGCGGC | | GAACCUGA | | CUGCGGAAC |
| | GGCC | | | | CUGU |
| 703 | UACAGGUUC | 893 | CGCUGCGGA | 1083 | GCCGCCGCU |
| | CGCAGCGGC | | ACCUGUA | | GCGGAACCU |
| | GGC | | | | GUU |
| 704 | UAACAGGUU | 894 | GCUGCGGAA | 1084 | CCGCCGCUG |
| | CCGCAGCGG | | CCUGUUA | | CGGAACCUG |
| | CGG | | | | UUC |
| 705 | UGAACAGGU | 895 | CUGCGGAAC | 1085 | CGCCGCUGC |
| | UCCGCAGCG | | CUGUUCA | | GGAACCUGU |
| | GCG | | | | UCA |
| 706 | UCAGCGCGU | 896 | GCCAAGCAC | 1086 | GGCUGGCCA |
| | GCUUGGCCA | | GCGCUGA | | AGCACGCGC |
| | GCC | | | | UGG |
| 707 | UCCAGCGCG | 897 | CCAAGCACG | 1087 | GCUGGCCAA |
| | UGCUUGGCC | | CGCUGGA | | GCACGCGCU |
| | AGC | | | | GGA |
| 708 | UUCCAGCGC | 898 | CAAGCACGC | 1088 | CUGGCCAAG |
| | GUGCUUGG | | GCUGGAA | | CACGCGCUG |
| | CCAG | | | | GAA |
| 709 | UAAUCAUGU | 899 | CUGGAAAAC | 1089 | ACGCGCUGG |
| | UUUCCAGCG | | AUGAUUA | | AAAACAUGA |
| | CGU | | | | UUA |
| 710 | UGUUGAACA | 900 | UCAGCCAUG | 1090 | CACAGUCAG |
| | UGGCUGACU | | UUCAACA | | CCAUGUUCA |
| | GUG | | | | ACC |
| 711 | UGGUUGAAC | 901 | CAGCCAUGU | 1091 | ACAGUCAGC |
| | AUGGCUGAC | | UCAACCA | | CAUGUUCAA |
| | UGU | | | | CCA |
| 712 | UUGGUUGAA | 902 | AGCCAUGUU | 1092 | CAGUCAGCC |
| | CAUGGCUGA | | CAACCAA | | AUGUUCAAC |
| | CUG | | | | CAG |
| 713 | UCUGGUUGA | 903 | GCCAUGUUC | 1093 | AGUCAGCCA |
| | ACAUGGCUG | | AACCAGA | | UGUUCAACC |
| | ACU | | | | AGG |
| 714 | UACCUGGUU | 904 | CAUGUUCAA | 1094 | UCAGCCAUG |
| | GAACAUGGC | | CCAGGUA | | UUCAACCAG |
| | UGA | | | | GUC |
| 715 | UGACCUGGU | 905 | AUGUUCAAC | 1095 | CAGCCAUGU |
| | UGAACAUGG | | CAGGUCA | | UCAACCAGG |
| | CUG | | | | UCC |
| 716 | UGGACCUGG | 906 | UGUUCAACC | 1096 | AGCCAUGUU |
| | UUGAACAUG | | AGGUCCA | | CAACCAGGU |
| | GCU | | | | CCU |
| 717 | UAGGACCUG | 907 | GUUCAACCA | 1097 | GCCAUGUUC |
| | GUUGAACAU | | GGUCCUA | | AACCAGGUC |
| | GGC | | | | CUC |
| 718 | UAGGAUGAG | 908 | CCAGGUCCU | 1098 | UUCAACCAG |
| | GACCUGGUU | | CAUCCUA | | GUCCUCAUC |
| | GAA | | | | CUC |
| 719 | UCAGGGUGC | 909 | CUCUUCUGC | 1099 | UCAUCCUCU |
| | AGAAGAGGA | | ACCCUGA | | UCUGCACCC |
| | UGA | | | | UGC |
| 720 | UCAGCAGGG | 910 | UUCUGCACC | 1100 | UCCUCUUCU |
| | UGCAGAAGA | | CUGCUGA | | GCACCCUGC |
| | GGA | | | | UGU |
| 721 | UAGAAGGAG | 911 | UCCUGACCU | 1101 | GUCCCUCCU |
| | GUCAGGAGG | | CCUUCUA | | GACCUCCUU |
| | GAC | | | | CUA |
| 722 | UGUAGAAGG | 912 | CUGACCUCC | 1102 | CCCUCCUGA |
| | AGGUCAGGA | | UUCUACA | | CCUCCUUCU |
| | GGG | | | | ACU |
| 723 | UAAGUAGAA | 913 | GACCUCCUU | 1103 | CUCCUGACC |
| | GGAGGUCAG | | CUACUUA | | UCCUUCUAC |
| | GAG | | | | UUC |
| 724 | UCAGAAGUA | 914 | CUCCUUCUA | 1104 | CUGACCUCC |
| | GAAGGAGGU | | CUUCUGA | | UUCUACUUC |
| | CAG | | | | UGC |
| 725 | UGACGAUGC | 915 | UACUUCUGC | 1105 | CCUUCUACU |
| | AGAAGUAGA | | AUCGUCA | | UCUGCAUCG |
| | AGG | | | | UCA |
| 726 | UUGACGAUG | 916 | ACUUCUGCA | 1106 | CUUCUACUU |
| | CAGAAGUAG | | UCGUCAA | | CUGCAUCGU |
| | AAG | | | | CAC |
| 727 | UGAUCAUGA | 917 | GUGGUCAUC | 1107 | UGCUGGUG |
| | UGACCACCA | | AUGAUCA | | GUCAUCAUG |
| | GCA | | | | AUCU |
| 728 | UGGAAGUCC | 918 | UUCUCAUGG | 1108 | CGACCUUCU |
| | AUGAGAAGG | | ACUUCCA | | CAUGGACUU |
| | UCG | | | | CCU |
| 729 | UAGGAAGUC | 919 | UCUCAUGGA | 1109 | GACCUUCUC |
| | CAUGAGAAG | | CUUCCUA | | AUGGACUUC |
| | GUC | | | | CUG |
| 730 | UGUUCAGGA | 920 | AUGGACUUC | 1110 | UUCUCAUGG |
| | AGUCCAUGA | | CUGAACA | | ACUUCCUGA |
| | GAA | | | | ACG |
| 731 | UCGUUCAGG | 921 | UGGACUUCC | 1111 | UCUCAUGGA |
| | AAGUCCAUG | | UGAACGA | | CUUCCUGAA |
| | AGA | | | | CGA |
| 732 | UUCGUUCAG | 922 | GGACUUCCU | 1112 | CUCAUGGAC |
| | GAAGUCCAU | | GAACGAA | | UUCCUGAAC |
| | GAG | | | | GAG |
| 733 | UGUAAUAGU | 923 | CUCCAGGAC | 1113 | CCCGGCUCC |
| | CCUGGAGCC | | UAUUACA | | AGGACUAUU |
| | GGG | | | | ACG |
| 734 | UAUGACCAC | 924 | CUAUUACGU | 1114 | CAGGACUAU |
| | GUAAUAGUC | | GGUCAUA | | UACGUGGUC |
| | CUG | | | | AUC |
| 735 | UUUCAGGUU | 925 | AUCCUCAAA | 1115 | UCCAGAUCC |
| | UGAGGAUCU | | CCUGAAA | | UCAAACCUG |
| | GGA | | | | AAA |
| 736 | UAACUUGUU | 926 | ACCUGAAAA | 1116 | CUCAAACCU |
| | UUCAGGUUU | | CAAGUUA | | GAAAACAAG |
| | GAG | | | | UUU |
| 737 | UAAACUUGU | 927 | CCUGAAAAC | 1117 | UCAAACCUG |
| | UUUCAGGUU | | AAGUUUA | | AAAACAAGU |
| | UGA | | | | UUC |
| 738 | UACACCACG | 928 | CUGACCACG | 1118 | GUUUGCUGA |
| | UGGUCAGCA | | UGGUGUA | | CCACGUGGU |
| | AAC | | | | GUG |
| 739 | UCAGAUGCA | 929 | GCUGAACUG | 1119 | CUGGCGCU |
| | GUUCAGCGC | | CAUCUGA | | GAACUGCAU |
| | CAG | | | | CUGC |
| 740 | UGAACUUGC | 930 | GGUGACAGC | 1120 | GCAUGGGU |
| | UGUCACCCA | | AAGUUCA | | GACAGCAAG |
| | UGC | | | | UUCU |
| 741 | UAGAACUUG | 931 | GUGACAGCA | 1121 | CAUGGGUGA |
| | CUGUCACCC | | AGUUCUA | | CAGCAAGUU |
| | AUG | | | | CUU |
| 742 | UAGCCCGAU | 932 | GUGCCUCAU | 1122 | GGCGUGUG |
| | GAGGCACAC | | CGGGCUA | | CCUCAUCGG |
| | GCC | | | | GCUG |
| 743 | UCUUCAGCC | 933 | CUCAUCGGG | 1123 | UGUGCCUCA |
| | CGAUGAGGC | | CUGAAGA | | UCGGGCUGA |
| | ACA | | | | AGC |
| 744 | UCCGCUUCA | 934 | AUCGGGCUG | 1124 | GCCUCAUCG |
| | GCCCGAUGA | | AAGCGGA | | GGCUGAAGC |
| | GGC | | | | GGG |
| 745 | UGAUGUAGA | 935 | ACCUGCUUC | 1125 | CUGACACCU |
| | AGCAGGUGU | | UACAUCA | | GCUUCUACA |
| | CAG | | | | UCA |
| 746 | UUGGUGAUG | 936 | ACAUCAACA | 1126 | CUUCUACAU |
| | UUGAUGUAG | | UCACCAA | | CAACAUCAC |
| | AAG | | | | CAA |
| 747 | UCAGCCCUU | 937 | GCUGGACAA | 1127 | CUGCGGCU |
| | GUCCAGCCG | | GGGCUGA | | GGACAAGGG |
| | CAG | | | | CUGC |
| 748 | UUUGUUCUU | 938 | CGGGUUCAA | 1128 | GCCUACGGG |
| | GAACCCGUA | | GAACAAA | | UUCAAGAAC |
| | GGC | | | | AAG |
| 749 | UCUUGUUCU | 939 | GGGUUCAAG | 1129 | CCUACGGGU |
| | UGAACCCGU | | AACAAGA | | UCAAGAACA |
| | AGG | | | | AGC |
| 750 | UGAUCAGCU | 940 | AAGAACAAG | 1130 | GGUUCAAGA |
| | UGUUCUUGA | | CUGAUCA | | ACAAGCUGA |
| | ACC | | | | UCA |
| 751 | UUGAUCAGC | 941 | AGAACAAGC | 1131 | GUUCAAGAA |
| | UUGUUCUUG | | UGAUCAA | | CAAGCUGAU |
| | AAC | | | | CAU |
| 752 | UAUGAUCAG | 942 | GAACAAGCU | 1132 | UUCAAGAAC |
| | CUUGUUCUU | | GAUCAUA | | AAGCUGAUC |
| | GAA | | | | AUC |
| 753 | UGGAUCUGU | 943 | GCCUACUAC | 1133 | UGCGGGCC |
| | AGUAGGCCC | | AGAUCCA | | UACUACAGA |
| | GCA | | | | UCCC |
| 754 | UGGGAUCUG | 944 | CCUACUACA | 1134 | GCGGGCCUA |
| | UAGUAGGCC | | GAUCCCA | | CUACAGAUC |
| | CGC | | | | CCG |
| 755 | UGAAGUGGU | 945 | CCCGACCAC | 1135 | ACAAGCCCG |
| | GGUCGGGC | | CACUUCA | | ACCACCACU |
| | UUGU | | | | UCC |
| 756 | UGGAAGUGG | 946 | CCGACCACC | 1136 | CAAGCCCGA |
| | UGGUCGGG | | ACUUCCA | | CCACCACUU |
| | CUUG | | | | CCU |
| 757 | UAGGAAGUG | 947 | CGACCACCA | 1137 | AAGCCCGAC |
| | GUGGUCGG | | CUUCCUA | | CACCACUUC |
| | GCUU | | | | CUG |
| 758 | UCAGGAAGU | 948 | GACCACCAC | 1138 | AGCCCGACC |
| | GGUGGUCG | | UUCCUGA | | ACCACUUCC |
| | GGCU | | | | UGG |
| 759 | UUUCCAGGA | 949 | CACCACUUC | 1139 | CCGACCACC |
| | AGUGGUGG | | CUGGAAA | | ACUUCCUGG |
| | UCGG | | | | AAG |
| 760 | UCUUCCAGG | 950 | ACCACUUCC | 1140 | CGACCACCA |
| | AAGUGGUGG | | UGGAAGA | | CUUCCUGGA |
| | UCG | | | | AGC |
| 761 | UGCUUCCAG | 951 | CCACUUCCU | 1141 | GACCACCAC |
| | GAAGUGGUG | | GGAAGCA | | UUCCUGGAA |
| | GUC | | | | GCC |
| 762 | UCUCUCCUU | 952 | GGUGGACAA | 1142 | GUGGUGGU |
| | GUCCACCAC | | GGAGAGA | | GGACAAGGA |
| | CAC | | | | GAGC |
| 763 | UUAGUUUGG | 953 | GCUCUUUCC | 1143 | CUCUGGCUC |
| | AAAGAGCCA | | AAACUAA | | UUUCCAAAC |
| | GAG | | | | UAG |
| 764 | UCUAGUUUG | 954 | CUCUUUCCA | 1144 | UCUGGCUCU |
| | GAAAGAGCC | | AACUAGA | | UUCCAAACU |
| | AGA | | | | AGA |
| 765 | UUCUAGUUU | 955 | UCUUUCCAA | 1145 | CUGGCUCUU |
| | GGAAAGAGC | | ACUAGAA | | UCCAAACUA |
| | CAG | | | | GAA |
| 766 | UUUCUAGUU | 956 | CUUUCCAAA | 1146 | UGGCUCUUU |
| | UGGAAAGAG | | CUAGAAA | | CCAAACUAG |
| | CCA | | | | AAA |
| 767 | UUUUCUAGU | 957 | UUUCCAAAC | 1147 | GGCUCUUUC |
| | UUGGAAAGA | | UAGAAAA | | CAAACUAGA |
| | GCC | | | | AAA |
| 768 | UCUCUUUUC | 958 | CAAACUAGA | 1148 | CUUUCCAAA |
| | UAGUUUGGA | | AAAGAGA | | CUAGAAAAG |
| | AAG | | | | AGG |
| 769 | UCCCUCUUU | 959 | AACUAGAAA | 1149 | UUCCAAACU |
| | UCUAGUUUG | | AGAGGGA | | AGAAAAGAG |
| | GAA | | | | GGA |
| 770 | UUCCCUCUU | 960 | ACUAGAAAA | 1150 | UCCAAACUA |
| | UUCUAGUUU | | GAGGGAA | | GAAAAGAGG |
| | GGA | | | | GAG |
| 771 | UGGCGGAAC | 961 | CCUUCAUGU | 1151 | CCUGGCCUU |
| | AUGAAGGCC | | UCCGCCA | | CAUGUUCCG |
| | AGG | | | | CCU |
| 772 | UCGGCAGGC | 962 | AUGUUCCGC | 1152 | CCUUCAUGU |
| | GGAACAUGA | | CUGCCGA | | UCCGCCUGC |
| | AGG | | | | CGU |
| 773 | UACGGCAGG | 963 | UGUUCCGCC | 1153 | CUUCAUGUU |
| | CGGAACAUG | | UGCCGUA | | CCGCCUGCC |
| | AAG | | | | GUU |
| 774 | UAACGGCAG | 964 | GUUCCGCCU | 1154 | UUCAUGUUC |
| | GCGGAACAU | | GCCGUUA | | CGCCUGCCG |
| | GAA | | | | UUC |
| 775 | UGAACGGCA | 965 | UUCCGCCUG | 1155 | UCAUGUUCC |
| | GGCGGAACA | | CCGUUCA | | GCCUGCCGU |
| | UGA | | | | UCG |
| 776 | UCUGAUGCU | 966 | CGUCUUCAG | 1156 | GGCCGCGU |
| | GAAGACGCG | | CAUCAGA | | CUUCAGCAU |
| | GCC | | | | CAGC |
| 777 | UGACUGGUA | 967 | ACUGCUCUA | 1157 | GACACACUG |
| | GAGCAGUGU | | CCAGUCA | | CUCUACCAG |
| | GUC | | | | UCC |
| 778 | UGGACUGGU | 968 | CUGCUCUAC | 1158 | ACACACUGC |
| | AGAGCAGUG | | CAGUCCA | | UCUACCAGU |
| | UGU | | | | CCU |
| 779 | UUCCUUCAC | 969 | GUCCUUCGU | 1159 | UACCAGUCC |
| | GAAGGACUG | | GAAGGAA | | UUCGUGAAG |
| | GUA | | | | GAC |
| 780 | UGUCCUUCA | 970 | UCCUUCGUG | 1160 | ACCAGUCCU |
| | CGAAGGACU | | AAGGACA | | UCGUGAAGG |
| | GGU | | | | ACU |
| 781 | UUAGUCCUU | 971 | CUUCGUGAA | 1161 | CAGUCCUUC |
| | CACGAAGGA | | GGACUAA | | GUGAAGGAC |
| | CUG | | | | UAC |
| 782 | UGUAGUCCU | 972 | UUCGUGAAG | 1162 | AGUCCUUCG |
| | UCACGAAGG | | GACUACA | | UGAAGGACU |
| | ACU | | | | ACA |
| 783 | UUGUAGUCC | 973 | UCGUGAAGG | 1163 | GUCCUUCGU |
| | UUCACGAAG | | ACUACAA | | GAAGGACUA |
| | GAC | | | | CAU |
| 784 | UGGGUGAU | 974 | UGAUCACCA | 1164 | CUACAUGAU |
| | GGUGAUCAU | | UCACCCA | | CACCAUCAC |
| | GUAG | | | | CCG |
| 785 | UCGGGUGAU | 975 | GAUCACCAU | 1165 | UACAUGAUC |
| | GGUGAUCAU | | CACCCGA | | ACCAUCACC |
| | GUA | | | | CGG |
| 786 | UCCGGGUGA | 976 | AUCACCAUC | 1166 | ACAUGAUCA |
| | UGGUGAUCA | | ACCCGGA | | CCAUCACCC |
| | UGU | | | | GGC |
| 787 | UGCCGGGU | 977 | UCACCAUCA | 1167 | CAUGAUCAC |
| | GAUGGUGAU | | CCCGGCA | | CAUCACCCG |
| | CAUG | | | | GCU |
| 788 | UCGGUGAUU | 978 | CCAUGAAAA | 1168 | CUGUGCCAU |
| | UUCAUGGCA | | UCACCGA | | GAAAAUCAC |
| | CAG | | | | CGA |
| 789 | UUUCACCGA | 979 | CCAGAUCUC | 1169 | CAGUCCCAG |
| | GAUCUGGGA | | GGUGAAA | | AUCUCGGUG |
| | CUG | | | | AAC |
| 790 | UGAGAGCUC | 980 | GCGCCAGGA | 1170 | UCUGAGCGC |
| | CUGGCGCUC | | GCUCUCA | | CAGGAGCUC |
| | AGA | | | | UCC |
| 791 | UGCGGUUCU | 981 | CUGGUGAAG | 1171 | CCGAGCUGG |
| | UCACCAGCU | | AACCGCA | | UGAAGAACC |
| | CGG | | | | GCA |
| 792 | UGGUCGUUC | 982 | ACGAGAUGA | 1172 | CUACGACGA |
| | AUCUCGUCG | | ACGACCA | | GAUGAACGA |
| | UAG | | | | CCA |
| 793 | UCCACGUCC | 983 | UCAGGAAGG | 1173 | GGUCCUCAG |
| | UUCCUGAGG | | ACGUGGA | | GAAGGACGU |
| | ACC | | | | GGA |
| 794 | UGUCCUCAC | 984 | GGAGCGUG | 1174 | GUGGAGGA |
| | ACGCUCCUC | | UGAGGACA | | GCGUGUGA |
| | CAC | | | | GGACA |
| 795 | UCCAGGGUG | 985 | GGGACUCCA | 1175 | CUCCUGGGA |
| | GAGUCCCAG | | CCCUGGA | | CUCCACCCU |
| | GAG | | | | GGA |
| 796 | UUCCAGGGU | 986 | GGACUCCAC | 1176 | UCCUGGGAC |
| | GGAGUCCCA | | CCUGGAA | | UCCACCCUG |
| | GGA | | | | GAA |
| 797 | UUUCCAGGG | 987 | GACUCCACC | 1177 | CCUGGGACU |
| | UGGAGUCCC | | CUGGAAA | | CCACCCUGG |
| | AGG | | | | AAA |
| 798 | UUUUCCAGG | 988 | ACUCCACCC | 1178 | CUGGGACUC |
| | GUGGAGUCC | | UGGAAAA | | CACCCUGGA |
| | CAG | | | | AAG |
| 799 | UACUCUCCU | 989 | GCUAGUGAG | 1179 | UGCAGGCUA |
| | CACUAGCCU | | GAGAGUA | | GUGAGGAGA |
| | GCA | | | | GUU |
| 800 | UAAACUCUC | 990 | UAGUGAGGA | 1180 | CAGGCUAGU |
| | CUCACUAGC | | GAGUUUA | | GAGGAGAGU |
| | CUG | | | | UUU |
| 801 | UGGACUGCA | 991 | ACGUCGAUG | 1181 | UUUACACGU |
| | UCGACGUGU | | CAGUCCA | | CGAUGCAGU |
| | AAA | | | | CCA |
| 802 | UACAUCUGU | 992 | UCUUUACAC | 1182 | ACUUCUCUU |
| | GUAAAGAGA | | AGAUGUA | | UACACAGAU |
| | AGU | | | | GUA |
| 803 | UGUACAUCU | 993 | UUUACACAG | 1183 | UUCUCUUUA |
| | GUGUAAAGA | | AUGUACA | | CACAGAUGU |
| | GAA | | | | ACC |
| 804 | UGCCCUGGU | 994 | ACUCGUGAC | 1184 | CCGCAACUC |
| | CACGAGUUG | | CAGGGCA | | GUGACCAGG |
| | CGG | | | | GCU |
| 805 | UCAGAAAGG | 995 | CCGAGGACC | 1185 | UGUCGCCGA |
| | UCCUCGGCG | | UUUCUGA | | GGACCUUUC |
| | ACA | | | | UGA |
| 806 | UUGGCGUCU | 996 | AAGCAGAAG | 1186 | GAAGGAAGC |
| | UCUGCUUCC | | ACGCCAA | | AGAAGACGC |
| | UUC | | | | CAU |
| 807 | UGAAAUGGC | 997 | AGAAGACGC | 1187 | GAAGCAGAA |
| | GUCUUCUGC | | CAUUUCA | | GACGCCAUU |
| | UUC | | | | UCC |
| 808 | UGGAAAUGG | 998 | GAAGACGCC | 1188 | AAGCAGAAG |
| | CGUCUUCUG | | AUUUCCA | | ACGCCAUUU |
| | CUU | | | | CCU |
| 809 | UAGAGGAAA | 999 | GACGCCAUU | 1189 | CAGAAGACG |
| | UGGCGUCUU | | UCCUCUA | | CCAUUUCCU |
| | CUG | | | | CUA |
| 810 | UUAGAGGAA | 1000 | ACGCCAUUU | 1190 | AGAAGACGC |
| | AUGGCGUCU | | CCUCUAA | | CAUUUCCUC |
| | UCU | | | | UAC |
| 811 | UAAGUAGAG | 1001 | CCAUUUCCU | 1191 | AGACGCCAU |
| | GAAAUGGCG | | CUACUUA | | UUCCUCUAC |
| | UCU | | | | UUC |
| 812 | UUGAAGUAG | 1002 | AUUUCCUCU | 1192 | ACGCCAUUU |
| | AGGAAAUGG | | ACUUCAA | | CCUCUACUU |
| | CGU | | | | CAC |
| 813 | UGUGUGAAG | 1003 | UCCUCUACU | 1193 | CCAUUUCCU |
| | UAGAGGAAA | | UCACACA | | CUACUUCAC |
| | UGG | | | | ACU |
| 814 | UUCAGUGUG | 1004 | UCUACUUCA | 1194 | UUUCCUCUA |
| | AAGUAGAGG | | CACUGAA | | CUUCACACU |
| | AAA | | | | GAA |
| 815 | UUUCAGUGU | 1005 | CUACUUCAC | 1195 | UUCCUCUAC |
| | GAAGUAGAG | | ACUGAAA | | UUCACACUG |
| | GAA | | | | AAC |
| 816 | UGAAAUGCA | 1006 | AAGAUGGUG | 1196 | GGCGGAAGA |
| | CCAUCUUCC | | CAUUUCA | | UGGUGCAUU |
| | GCC | | | | UCC |
| 817 | UGGAAAUGC | 1007 | AGAUGGUGC | 1197 | GCGGAAGAU |
| | ACCAUCUUC | | AUUUCCA | | GGUGCAUUU |
| | CGC | | | | CCA |
| 818 | UGGUCCAUG | 1008 | GCAUUUCCA | 1198 | AUGGUGCAU |
| | GAAAUGCAC | | UGGACCA | | UUCCAUGGA |
| | CAU | | | | CCA |
| 819 | UUAAGUGUA | 1009 | ACCAUUUUA | 1199 | CAUGGACCA |
| | AAAUGGUCC | | CACUUAA | | UUUUACACU |
| | AUG | | | | UAC |
| 820 | UGUAAGUGU | 1010 | CCAUUUUAC | 1200 | AUGGACCAU |
| | AAAAUGGUC | | ACUUACA | | UUUACACUU |
| | CAU | | | | ACC |
| 821 | UGGUAAGUG | 1011 | CAUUUUACA | 1201 | UGGACCAUU |
| | UAAAAUGGU | | CUUACCA | | UUACACUUA |
| | CCA | | | | CCU |
| 822 | UAGGUAAGU | 1012 | AUUUUACAC | 1202 | GGACCAUUU |
| | GUAAAAUGG | | UUACCUA | | UACACUUAC |
| | UCC | | | | CUU |
| 823 | UAAAGGUAA | 1013 | UUUACACUU | 1203 | ACCAUUUUA |
| | GUGUAAAAU | | ACCUUUA | | CACUUACCU |
| | GGU | | | | UUU |
| 824 | UAAAAGGUA | 1014 | UUACACUUA | 1204 | CCAUUUUAC |
| | AGUGUAAAA | | CCUUUUA | | ACUUACCUU |
| | UGG | | | | UUA |
| 825 | UUAAAAGGU | 1015 | UACACUUAC | 1205 | CAUUUUACA |
| | AAGUGUAAA | | CUUUUAA | | CUUACCUUU |
| | AUG | | | | UAA |
| 826 | UUUAAAAGG | 1016 | ACACUUACC | 1206 | AUUUUACAC |
| | UAAGUGUAA | | UUUUAAA | | UUACCUUUU |
| | AAU | | | | AAA |
| 827 | UUUUAAAAG | 1017 | CACUUACCU | 1207 | UUUUACACU |
| | GUAAGUGUA | | UUUAAAA | | UACCUUUUA |
| | AAA | | | | AAG |
| 828 | UUGCUUUAA | 1018 | UUACCUUUU | 1208 | UACACUUAC |
| | AAGGUAAGU | | AAAGCAA | | CUUUUAAAG |
| | GUA | | | | CAA |
| 829 | UUUGCUUUA | 1019 | UACCUUUUA | 1209 | ACACUUACC |
| | AAAGGUAAG | | AAGCAAA | | UUUUAAAGC |
| | UGU | | | | AAA |
| 830 | UUUUGCUUU | 1020 | ACCUUUUAA | 1210 | CACUUACCU |
| | AAAAGGUAA | | AGCAAAA | | UUUAAAGCA |
| | GUG | | | | AAG |
| 831 | UCUUUGCUU | 1021 | CCUUUUAAA | 1211 | ACUUACCUU |
| | UAAAAGGUA | | GCAAAGA | | UUAAAGCAA |
| | AGU | | | | AGC |
| 832 | UGCUUUGCU | 1022 | CUUUUAAAG | 1212 | CUUACCUUU |
| | UUAAAAGGU | | CAAAGCA | | UAAAGCAAA |
| | AAG | | | | GCC |
| 833 | UAGGCUUUG | 1023 | UUUAAAGCA | 1213 | UACCUUUUA |
| | CUUUAAAAG | | AAGCCUA | | AAGCAAAGC |
| | GUA | | | | CUC |
| 834 | UAUGAGGCU | 1024 | AAAGCAAAG | 1214 | CUUUUAAAG |
| | UUGCUUUAA | | CCUCAUA | | CAAAGCCUC |
| | AAG | | | | AUU |
| 835 | UAAAAUGAG | 1025 | GCAAAGCCU | 1215 | UUAAAGCAA |
| | GCUUUGCUU | | CAUUUUA | | AGCCUCAUU |
| | UAA | | | | UUC |
| 836 | UGGGAUCUC | 1026 | GCAAGGCGA | 1216 | UCUCAGCAA |
| | GCCUUGCUG | | GAUCCCA | | GGCGAGAUC |
| | AGA | | | | CCG |
| 837 | UAGCAAAGG | 1027 | CCUGCGUCC | 1217 | GCCACCCUG |
| | ACGCAGGGU | | UUUGCUA | | CGUCCUUUG |
| | GGC | | | | CUG |
| 838 | UAAGGAACC | 1028 | UGCUGCUG | 1218 | UCCUUUGCU |
| | AGCAGCAAA | | GUUCCUUA | | GCUGGUUCC |
| | GGA | | | | UUA |
| 839 | UCAGUAAGG | 1029 | GCUGGUUCC | 1219 | UUGCUGCUG |
| | AACCAGCAG | | UUACUGA | | GUUCCUUAC |
| | CAA | | | | UGG |
| 840 | UGCUGACCG | 1030 | GUUUGUACG | 1220 | UACUGGUUU |
| | UACAAACCA | | GUCAGCA | | GUACGGUCA |
| | GUA | | | | GCG |
| 841 | UAUAGAAGU | 1031 | GCUGGAAAC | 1221 | UCAGCGCUG |
| | UUCCAGCGC | | UUCUAUA | | GAAACUUCU |
| | UGA | | | | AUU |
| 842 | UAAUAGAAG | 1032 | CUGGAAACU | 1222 | CAGCGCUGG |
| | UUUCCAGCG | | UCUAUUA | | AAACUUCUA |
| | CUG | | | | UUA |
| 843 | UUUUAAUAG | 1033 | GAAACUUCU | 1223 | CGCUGGAAA |
| | AAGUUUCCA | | AUUAAAA | | CUUCUAUUA |
| | GCG | | | | AAU |
| 844 | UAGAAUGCA | 1034 | AAAUGGAUG | 1224 | CUAUUAAAU |
| | UCCAUUUAA | | CAUUCUA | | GGAUGCAUU |
| | UAG | | | | CUG |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl(2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 564,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′- to -3′ direction

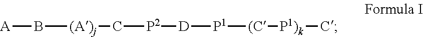

wherein A is represented by the formula C-P¹-D-P¹; each A′ is represented by the formula C-P²-D-P²; B is represented by the formula C-P²-D-P²-D-P²-D-P²; each C is a 2′-O-methyl(2′—O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′- to -3′ direction:

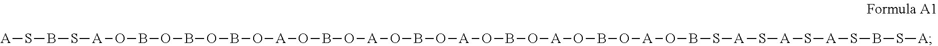

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′- to -3′ direction:

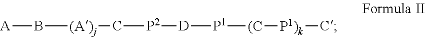

wherein A is represented by the formula C-P¹-D-P¹; each A′ is represented by the formula C-P²-D-P²; B is represented by the formula C-P²-D-P²-D-P²-D-P²; each C is a 2′-O-methyl(2′—O—Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′- to -3′ direction:

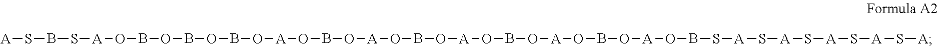

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′- to -3′ direction:

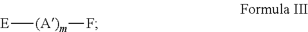

wherein E is represented by the formula (C-P¹)₂; F is represented by the formula (C-P²) 3-D-P¹-C-P¹-C, (C-P²) 3-D-P²-C-P²-C, (C-P²) 3-D-P¹-C-P¹-D, or (C-P²) 3-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′- to -3′ direction:

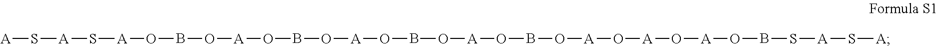

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′- to -3′ direction:

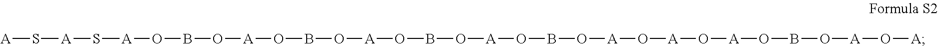

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′- to -3′ direction:

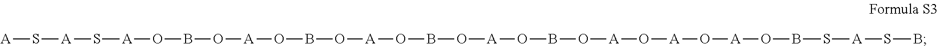

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′- to -3′ direction:

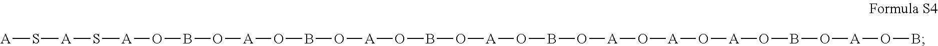

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′- to -3′ direction:

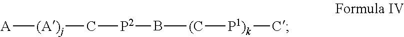

wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′- to -3′ direction:

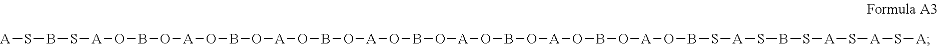

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′- to -3′ direction:

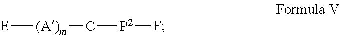

wherein E is represented by the formula (C-P¹)₂; F is represented by the formula D-P¹-C-P¹-C, D-P²-C-P²-C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′- to -3′ direction:

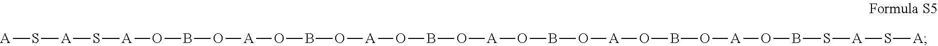

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′- to -3′ direction:

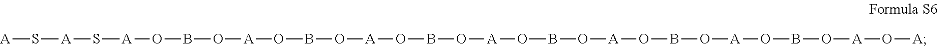

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′- to -3′ direction:

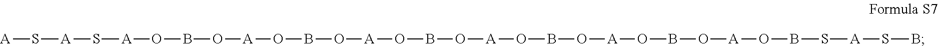

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′- to -3′ direction:

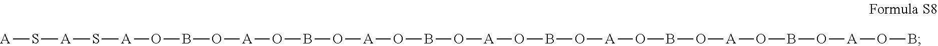

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′- to -3′ direction:

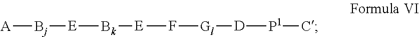

wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′- to -3′ direction:

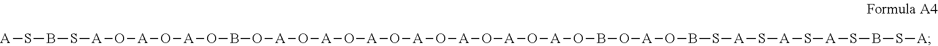

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′- to -3′ direction:

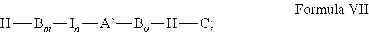

wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′- to -3′ direction:

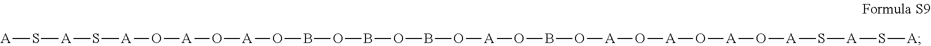

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

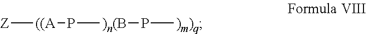

wherein Z is a 5′ phosphorus stabilizing moiety; each A is a 2′-O-methyl(2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

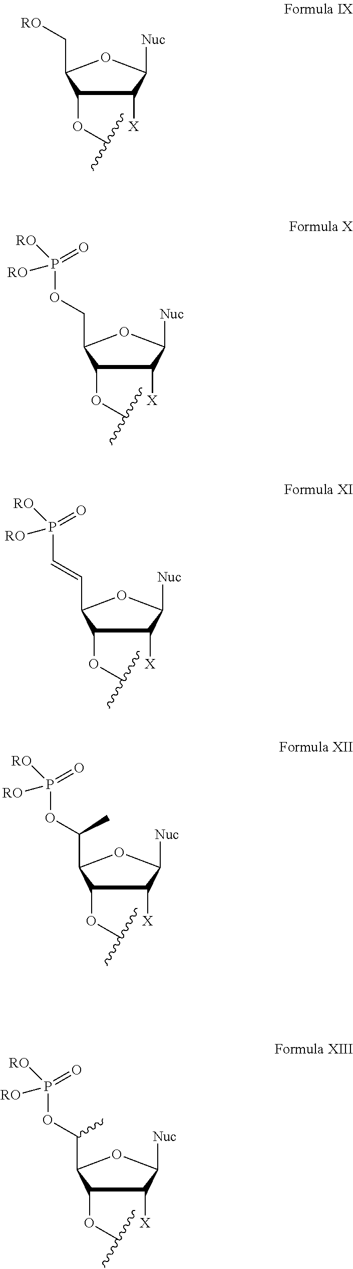

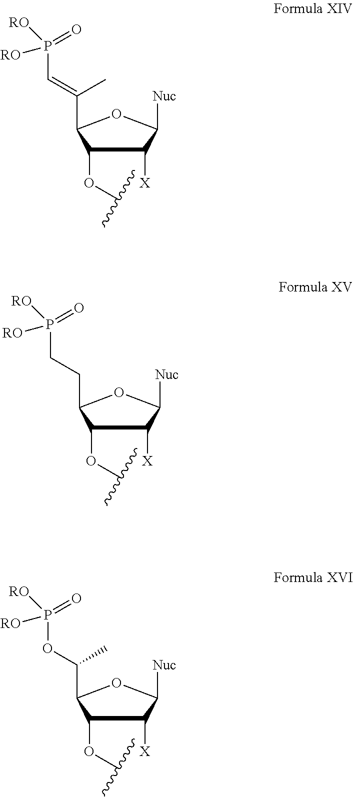

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substitued phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| 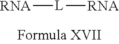 | 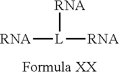 | 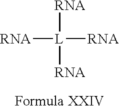 |
| 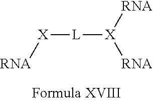 | 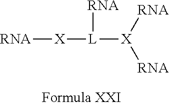 | 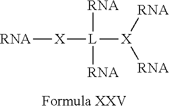 |
| 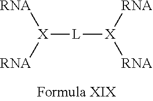 | 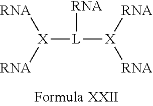 | 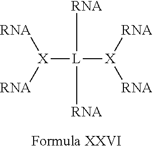 |
| | 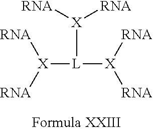 | 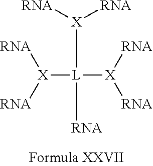 |
| | | 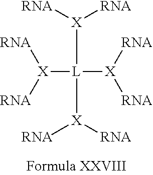 |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a polyethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

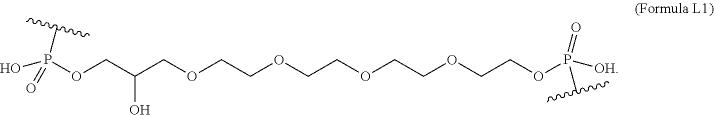 [SMILES: *P(=O)(O)OCCOCCOCCOCCOCC(O)COP(*)(=O)O]

In some embodiments, the linker has a structure of Formula L2:

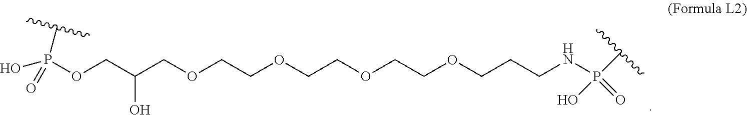 [SMILES: *P(=O)(O)NCCCOCCOCCOCCOCC(O)COP(*)(=O)O]

In some embodiments, the linker has a structure of Formula L3:

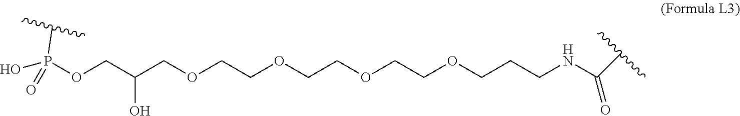 [SMILES: *C(=O)NCCCOCCOCCOCCOCC(O)COP(*)(=O)O]

In some embodiments, the linker has a structure of Formula L4:

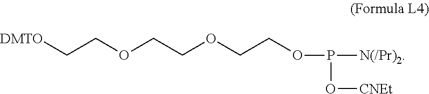

In some embodiments, the linker has a structure of Formula L5:

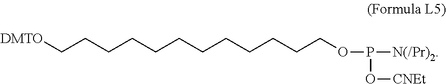

In some embodiments, the linker has a structure of Formula L6:

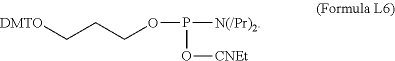

In some embodiments, the linker has a structure of Formula L7, as is shown below:

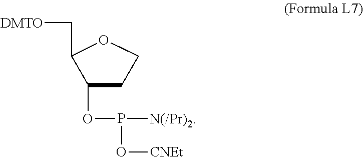

In some embodiments, the linker has a structure of Formula L8:

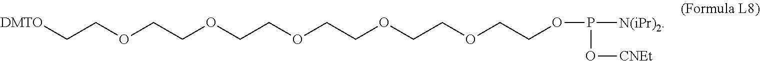

In some embodiments, the linker has a structure of Formula L9:

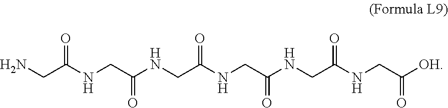 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The KCNT1-targeting siRNA molecules of the disclosure may be delivered to a subject, for example, as an anticonvulsant, thereby reducing the duration or frequency of epileptic events and/or mitigating epileptic-associated phenotypes (e.g., seizures, perioral cyanosis, flushing, apnea, hypertonia, pulmonary hemorrhage, or cardiac arrythmia). Furthermore, the siRNA molecules of the disclosure may also be delivered to a subject having a variant of the KCNT1 gene for which siRNA-mediated gene silencing of the KCNT1 variant gene reduces the expression level of KCNT1 transcript, thereby reducing the duration or frequency of epileptic events and/or mitigating epileptic-associated phenotypes (e.g., seizures, perioral cyanosis, flushing, apnea, hypertonia, pulmonary hemorrhage, or cardiac arrythmia).

Many pathological KCNT1 variants that drive epilepsy cause an increase in function, manifested as larger potassium ion currents through KCNT1 channels. Accordingly, decreasing KCNT1 expression levels pharmacologically should provide therapeutic benefit. The disclosure provides methods of treating a subject by way of KCNT1 gene silencing with one or more of the siRNA molecules described herein. The gene silencing may be performed in a subject to silence wild type KCNT1 transcripts, mutant KCNT1 transcripts, splice isoforms of KCNT1 transcripts, and/or overexpressed KCNT1 transcripts thereof, relative to a healthy subject. The method may include delivering to the CNS of the subject (e.g., a human) the siRNA molecules of the disclosure or a pharmaceutical composition containing the same by any appropriate route of administration (e.g., intracerebroventricular, intrathecal injection, or by intra-cisterna magna injection by catheterization). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, epilepsy, epilepsy-associated phenotypes (e.g., seizures, perioral cyanosis, flushing, apnea, hypertonia, pulmonary hemorrhage, or cardiac arrythmia), and/or any other medical risk(s) associated with a gain of function mutation in the KCNT1 gene. Additionally, subjects in need of treatment may be characterized as having a specific epilepsy syndrome. For example, the subject may be diagnosed with frontal lobe epilepsy, occipital lobe epilepsy, medial temporal lobe epilepsy, parietal lobe epilepsy, benign myoclonic epilepsy in infants, juvenile myoclonic epilepsy, childhood absence epilepsy, juvenile absence epilepsy, epilepsy with generalized tonic clonic seizures in childhood, infantile spasms, Lennox-Gastaut syndrome, West syndrome, sleep-related hypermotor epilepsy, progressive myoclonus epilepsies, febrile fits, epilepsy with continuous spike and waves in slow wave sleep, Laudau Kleffner syndrome, Rasmussen's syndrome, epilepsy arising from an inborn error in metabolism, epilepsy of infancy with migrating focal seizures, autosomal dominant nocturnal frontal lobe epilepsy, Ohtahara syndrome, early myoclonic encephalopathy, focal epilepsy, and/or multifocal epilepsy. Subjects that may be treated with the siRNA molecules disclosed herein may include, for example, humans, monkeys, rats, mice, pigs, and other mammals containing at least one orthologous copy of the KCNT1 gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular injection, intrastriatal injection, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Knockdown of KCNT1 with siRNA Molecules of the Disclosure

All screening assays were performed by measuring mRNA levels of KCNT1 and a housekeeping gene (ATP5B) in cell culture lysates via reverse transcription-quantitative polymerase chain reaction (RT-qPCR), using standard reagents and Applied Biosystems TaqMan Assays. For the passive screen (Table 3, below), SK-N-BE (2) cells were cultured in the presence of KCNT1 siRNA at either 2 μM or 0.5 μM concentration. For the active screen (Table 4, below), HEK cells stably expressing KCNT1 (HEK-KCNT1) were actively transfected with KCNT1 siRNA at either 20 nM or 0.2 nM concentration. For compound potency analysis, HEK-KCNT1 cells were actively transfected with KCNT1 siRNA at concentrations of 10 fM to 100 nM and the IC50 was calculated. For all assays, cells were dosed with siRNA for 72 hours prior to lysis and downstream analysis. Results are presented as the percent residual KCNT1 mRNA relative to untreated control cells in the same assay (% UNT KCNT1 mRNA). STDEV=standard deviation, ND=not determined.

TABLE 3
KCNT1 Knockdown with siRNA molecules
of the disclosure in a passive screen

| | | % UNT | | % UNT | |
|---|---|---|---|---|---|
| | | KCNT1 | | KCNT1 | |
| Antisense | Sense | mRNA | | mRNA | |
| SEQ | SEQ | expression | | expression | |
| ID NO: | ID NO: | @ 2 uM | STDEV | @ 0.5 uM | STDEV |
| 1 | 219 | 74.98 | 1.25 | 80.64 | 10.07 |
| 2 | 220 | 70.06 | 0.32 | 82.48 | 0.68 |
| 3 | 221 | 70.62 | 2.74 | 87.30 | 2.57 |
| 4 | 222 | 69.09 | 0.63 | 88.36 | 3.68 |
| 5 | 223 | 54.47 | 1.33 | 71.50 | 3.08 |
| 6 | 224 | 66.00 | 1.58 | 74.82 | 1.92 |
| 7 | 225 | 56.17 | 2.22 | 70.32 | 3.04 |
| 8 | 226 | 62.54 | 1.10 | 75.93 | 3.69 |
| 9 | 227 | 63.08 | 3.55 | 71.77 | 3.29 |
| 10 | 228 | 58.14 | 0.51 | 68.63 | 4.77 |
| 11 | 229 | 56.24 | 5.75 | 60.96 | 2.72 |
| 12 | 230 | 60.31 | 0.88 | 75.71 | 2.89 |
| 13 | 231 | 72.87 | 2.88 | 88.94 | 1.02 |
| 14 | 232 | 57.36 | 1.86 | 82.06 | 4.92 |
| 15 | 233 | 48.51 | 4.13 | 73.40 | 6.23 |
| 16 | 234 | 56.71 | 2.44 | 80.92 | 0.68 |
| 17 | 235 | 62.37 | 2.65 | 72.77 | 2.16 |
| 18 | 236 | 58.59 | 2.96 | 76.18 | 1.90 |
| 19 | 237 | 48.39 | 2.15 | 67.31 | 3.04 |
| 20 | 238 | 91.39 | 9.23 | 91.73 | 2.41 |
| 21 | 239 | 72.14 | 5.23 | 68.44 | 4.46 |
| 22 | 240 | 63.45 | 6.65 | 70.23 | 2.40 |
| 24 | 242 | 73.86 | 0.08 | 90.41 | 1.21 |
| 28 | 246 | 66.38 | 4.04 | 89.74 | 1.54 |
| 29 | 247 | 59.06 | 3.80 | 75.35 | 7.36 |
| 30 | 248 | 61.65 | 5.42 | 81.08 | 0.82 |
| 31 | 249 | 54.71 | 11.09 | 72.67 | 4.17 |
| 32 | 250 | 63.46 | 8.27 | 87.52 | 2.59 |
| 33 | 251 | 54.92 | 0.50 | 84.31 | 7.03 |
| 34 | 252 | 65.19 | 5.12 | 97.48 | 10.15 |
| 35 | 253 | 71.01 | 10.49 | 122.27 | ND |
| 36 | 254 | 82.87 | 2.76 | 97.08 | 10.50 |
| 37 | 255 | 70.67 | 4.62 | 98.42 | 10.69 |
| 38 | 256 | 58.81 | 2.41 | 82.23 | 5.19 |
| 39 | 257 | 75.15 | 3.12 | 97.43 | 6.49 |
| 40 | 258 | 84.62 | 5.29 | 88.19 | 1.25 |
| 41 | 259 | 49.57 | 2.81 | 82.98 | 1.51 |
| 42 | 260 | 58.43 | 1.24 | 66.94 | 6.58 |
| 43 | 261 | 67.23 | 0.28 | 88.21 | 3.16 |
| 44 | 262 | 79.76 | 4.06 | 90.78 | 1.30 |
| 45 | 263 | 64.06 | 7.83 | 79.15 | 12.64 |
| 46 | 264 | 74.63 | 5.61 | 90.93 | 10.23 |
| 47 | 265 | 70.32 | 2.78 | 87.55 | 2.39 |
| 48 | 266 | 80.31 | 2.15 | 96.56 | 3.09 |
| 49 | 267 | 80.73 | 2.25 | 101.59 | 3.24 |
| 50 | 268 | 76.35 | 2.19 | 86.04 | 0.86 |
| 51 | 269 | 65.55 | 0.32 | 78.90 | 2.77 |
| 52 | 270 | 61.85 | 4.39 | 86.84 | 2.08 |
| 53 | 271 | 59.62 | 0.60 | 86.18 | 4.87 |
| 54 | 272 | 72.51 | 4.09 | 86.19 | 2.97 |
| 55 | 273 | 74.82 | 4.90 | 96.32 | 5.11 |
| 56 | 274 | 68.93 | 1.78 | 80.83 | 10.98 |
| 57 | 275 | 80.49 | 18.79 | 64.50 | 4.95 |
| 58 | 276 | 69.02 | 7.73 | 66.57 | 2.09 |
| 59 | 277 | 80.40 | 7.71 | 77.02 | 1.79 |
| 60 | 278 | 75.78 | 3.55 | 83.44 | 5.25 |
| 61 | 279 | 58.29 | 30.15 | 108.66 | 3.20 |
| 62 | 280 | 70.60 | 5.27 | 82.29 | 8.45 |
| 63 | 281 | 102.81 | 1.00 | 102.28 | 0.05 |
| 64 | 282 | 97.44 | 0.49 | 113.21 | 13.66 |
| 65 | 283 | 106.57 | 6.42 | 118.17 | 6.89 |
| 66 | 284 | 100.33 | 9.06 | 105.80 | 4.41 |
| 67 | 285 | 98.37 | 8.09 | 76.09 | 2.69 |
| 68 | 286 | 65.96 | 0.39 | 88.92 | 4.20 |
| 69 | 287 | 71.68 | 6.44 | 99.43 | 7.16 |
| 70 | 288 | 80.83 | 2.13 | 95.37 | 4.76 |
| 71 | 289 | 62.52 | 1.51 | 82.85 | 6.91 |
| 72 | 290 | 69.34 | 5.50 | 90.11 | 1.02 |
| 73 | 291 | 81.34 | 2.50 | 95.31 | 5.71 |
| 86 | 304 | 88.73 | 13.44 | 103.78 | 5.52 |
| 87 | 305 | 90.23 | 18.07 | 107.98 | 0.28 |
| 88 | 306 | 89.79 | 6.96 | 104.43 | 5.40 |
| 89 | 307 | 71.47 | 2.47 | 78.04 | 4.64 |
| 90 | 308 | 72.25 | 8.63 | 96.37 | 4.92 |
| 91 | 309 | 77.49 | 0.97 | 94.06 | 4.59 |
| 92 | 310 | 70.61 | 2.23 | 82.79 | 14.06 |
| 93 | 311 | 65.84 | 5.17 | 95.80 | 3.99 |
| 94 | 312 | 82.72 | 15.55 | 91.67 | 0.61 |
| 96 | 314 | 66.28 | 0.47 | 88.28 | 1.68 |
| 98 | 316 | 76.57 | 0.27 | 103.01 | 0.46 |
| 99 | 317 | 69.15 | 5.75 | 83.34 | 5.96 |
| 100 | 318 | 80.66 | 0.94 | 99.11 | 3.91 |
| 101 | 319 | 77.06 | 0.34 | 94.91 | 0.39 |
| 102 | 320 | 69.85 | 0.38 | 93.29 | 0.82 |
| 103 | 321 | 112.43 | 4.95 | 106.95 | 4.12 |
| 104 | 322 | 95.79 | 1.75 | 100.46 | 1.16 |
| 105 | 323 | 74.47 | 1.13 | 106.33 | 6.09 |
| 106 | 324 | 70.13 | 0.96 | 94.72 | 3.29 |
| 107 | 325 | 90.76 | 1.89 | 97.64 | 0.61 |
| 108 | 326 | 91.68 | 1.67 | 108.23 | 5.77 |
| 109 | 327 | 148.92 | 16.98 | 109.74 | 7.15 |
| 110 | 328 | 95.45 | 0.63 | 106.31 | 3.97 |
| 111 | 329 | 83.30 | 3.29 | 98.75 | 2.04 |
| 112 | 330 | 77.98 | 5.45 | 96.97 | 4.02 |
| 113 | 331 | 195.39 | 25.60 | 126.49 | 0.30 |
| 114 | 332 | 87.94 | 0.45 | 104.09 | 1.72 |
| 115 | 333 | 69.63 | 3.50 | 96.37 | 1.56 |
| 116 | 334 | 90.84 | 2.66 | 96.18 | 0.75 |
| 117 | 335 | 73.66 | 2.09 | 97.86 | 2.18 |
| 118 | 336 | 83.04 | 3.33 | 104.29 | 7.00 |
| 119 | 337 | 79.33 | 2.67 | 109.47 | 1.03 |
| 120 | 338 | 82.01 | 0.43 | 106.43 | 1.80 |
| 121 | 339 | 70.51 | 2.15 | 98.78 | 2.83 |
| 122 | 340 | 64.60 | 1.29 | 95.18 | 0.57 |
| 123 | 341 | 97.26 | 6.25 | 78.62 | 6.09 |
| 127 | 345 | 91.78 | 12.93 | 83.25 | 9.73 |
| 128 | 346 | 73.30 | 7.49 | 109.32 | 7.18 |
| 129 | 347 | 92.92 | 13.32 | 110.86 | 12.57 |
| 130 | 348 | 87.34 | 1.84 | 99.02 | 3.55 |
| 131 | 349 | 83.35 | 1.26 | 96.41 | 1.98 |
| 132 | 350 | 82.55 | 12.14 | 85.24 | 9.72 |
| 133 | 351 | 66.86 | 0.11 | 75.18 | 8.69 |
| 134 | 352 | 56.40 | 1.97 | 78.68 | 5.03 |
| 135 | 353 | 60.47 | 0.10 | 72.54 | 2.54 |
| 136 | 354 | 65.57 | 5.67 | 78.59 | 9.12 |
| 137 | 355 | 74.76 | 1.06 | 95.31 | 2.75 |
| 138 | 356 | 59.74 | 24.46 | 89.58 | 7.26 |
| 139 | 357 | 59.90 | 0.06 | 103.66 | 3.76 |
| 140 | 358 | 144.53 | 6.22 | 115.67 | 0.38 |
| 141 | 359 | 79.97 | 4.06 | 94.81 | 5.12 |
| 142 | 360 | 73.09 | 4.80 | 79.10 | 2.25 |
| 143 | 361 | 72.96 | 4.40 | 110.27 | 6.05 |
| 144 | 362 | 67.88 | 7.44 | 96.13 | 6.85 |
| 145 | 363 | 62.34 | 4.42 | 86.70 | 6.14 |
| 146 | 364 | 71.12 | 9.08 | 85.53 | 2.57 |
| 147 | 365 | 67.32 | 2.91 | 93.31 | 1.25 |
| 148 | 366 | 84.88 | 0.38 | 91.61 | 4.31 |
| 149 | 367 | 75.70 | 1.85 | 98.57 | 8.01 |
| 150 | 368 | 96.60 | 1.51 | 84.09 | 1.48 |
| 151 | 369 | 67.54 | 4.24 | ND | ND |
| 152 | 370 | 95.14 | 15.91 | 86.26 | 3.60 |
| 153 | 371 | 84.51 | 6.75 | 92.36 | 10.47 |
| 154 | 372 | 98.08 | 9.13 | 91.24 | 1.94 |
| 155 | 373 | 80.29 | 14.00 | 103.38 | 3.20 |
| 156 | 374 | 82.71 | 4.67 | 103.49 | 5.64 |
| 157 | 375 | 90.55 | 7.12 | 98.98 | 3.75 |
| 158 | 376 | 90.02 | 8.87 | 105.89 | 7.41 |
| 162 | 380 | 113.46 | 13.78 | 96.17 | 18.73 |
| 163 | 381 | 97.19 | 7.32 | 98.74 | 17.40 |
| 164 | 382 | 113.15 | 10.24 | 99.15 | 4.26 |
| 165 | 383 | 87.49 | 2.07 | 90.22 | 10.15 |
| 166 | 384 | 84.38 | 9.74 | 92.48 | 2.45 |
| 167 | 385 | 75.66 | 4.16 | 90.57 | 1.96 |
| 169 | 387 | 87.08 | 7.19 | 103.40 | 2.40 |
| 170 | 388 | 81.29 | 8.99 | 102.94 | 5.48 |
| 171 | 389 | 111.74 | 6.19 | 101.33 | 0.02 |
| 172 | 390 | 80.28 | 1.27 | 112.53 | 2.83 |
| 173 | 391 | 80.62 | 4.86 | 100.60 | 0.90 |
| 174 | 392 | 90.57 | 6.27 | 85.70 | 2.19 |
| 175 | 393 | 99.03 | 0.30 | 86.37 | 9.04 |
| 176 | 394 | 88.27 | 0.19 | 65.92 | 8.13 |
| 177 | 395 | 87.61 | 12.58 | 82.94 | 4.23 |
| 178 | 396 | 106.70 | 15.95 | 81.20 | 1.47 |
| 179 | 397 | 90.15 | 4.33 | 82.90 | 9.56 |
| 180 | 398 | 92.38 | 2.75 | 86.07 | 0.31 |
| 181 | 399 | 71.94 | 2.54 | 71.63 | 13.00 |
| 182 | 400 | 76.27 | 2.26 | 77.93 | 7.78 |
| 183 | 401 | 71.18 | 4.10 | 90.08 | 2.40 |
| 184 | 402 | 66.84 | 8.13 | 99.51 | 1.18 |
| 185 | 403 | 61.73 | 0.04 | 92.54 | 5.55 |
| 186 | 404 | 61.34 | 1.29 | 101.22 | 2.97 |
| 187 | 405 | 55.50 | 1.31 | 95.55 | 5.52 |
| 188 | 406 | 53.24 | 1.43 | 107.19 | 6.01 |
| 189 | 407 | 57.43 | 1.02 | 98.28 | 0.24 |
| 190 | 408 | 60.82 | 2.68 | 93.81 | 4.18 |
| 191 | 409 | 68.55 | ND | 85.47 | 3.77 |
| 192 | 410 | 75.80 | 4.18 | 87.36 | 1.12 |
| 193 | 411 | 70.32 | 2.35 | 89.17 | 0.15 |
| 195 | 413 | 58.88 | 2.24 | 92.64 | 1.15 |
| 196 | 414 | 43.37 | 2.55 | 87.70 | 1.02 |
| 197 | 415 | 46.22 | 1.67 | 93.29 | 3.13 |
| 198 | 416 | 37.58 | 1.21 | 85.69 | 9.76 |
| 199 | 417 | 43.73 | 2.78 | 89.76 | 9.14 |
| 200 | 418 | 55.75 | 2.37 | 99.37 | 4.34 |
| 201 | 419 | 57.30 | 0.85 | 95.06 | 3.96 |
| 202 | 420 | 53.22 | 2.70 | 77.16 | 4.92 |
| 203 | 421 | 90.15 | 0.36 | 101.16 | 0.10 |
| 204 | 422 | 65.44 | 2.87 | 91.48 | 3.06 |
| 205 | 423 | 66.94 | 2.76 | 92.80 | 4.49 |
| 206 | 424 | 64.80 | 3.90 | 103.87 | 0.49 |
| 207 | 425 | 74.78 | 1.96 | 98.32 | 8.27 |
| 208 | 426 | 81.68 | 2.12 | 100.12 | 0.66 |
| 209 | 427 | 76.30 | 0.76 | 78.24 | 3.56 |
| 210 | 428 | 105.27 | 1.91 | 85.97 | 6.58 |
| 211 | 429 | 117.48 | 4.70 | 87.41 | 1.39 |
| 212 | 430 | 97.28 | 1.26 | 91.71 | 7.71 |
| 213 | 431 | 91.49 | 7.57 | 95.61 | 1.46 |
| 214 | 432 | 155.17 | 63.85 | 103.05 | 0.01 |
| 215 | 433 | 99.66 | 6.64 | 100.20 | 9.29 |
| 216 | 434 | 99.65 | 8.17 | 94.26 | 2.46 |
| 217 | 435 | 122.16 | 3.68 | 101.47 | 1.63 |
| 218 | 436 | 121.76 | 1.17 | 103.56 | 0.04 |

TABLE 4
KCNT1 Knockdown with siRNA molecules
of the disclosure in an active screen

| | | % UNT | | % UNT | |
|---|---|---|---|---|---|
| | | KCNT1 | | KCNT1 | |
| Antisense | Sense | mRNA | | mRNA | |
| SEQ | SEQ | expression | | expression | |
| ID NO: | ID NO: | @ 20 nM | STDEV | @ 0.2 nM | STDEV |
| 124 | 342 | 84.48 | 0.71 | 48.79 | 1.51 |
| 194 | 412 | 65.24 | 0.33 | 21.93 | 0.35 |
| 655 | 845 | 116.15 | 3.18 | 80.63 | 14.17 |
| 656 | 846 | 67.17 | 4.22 | 59.88 | 8.89 |
| 657 | 847 | 95.53 | 0.13 | 67.07 | 7.05 |
| 658 | 848 | 87.71 | 4.38 | 65.26 | 7.35 |
| 659 | 849 | 39.58 | 0.56 | 52.83 | 0.63 |
| 660 | 850 | 24.39 | 0.46 | 76.27 | 1.94 |
| 661 | 851 | 37.16 | 0.53 | 61.78 | 1.04 |
| 662 | 852 | 39.31 | 1.37 | 81.23 | 5.35 |
| 663 | 853 | 24.51 | 0.23 | 33.75 | 1.94 |
| 664 | 854 | 84.55 | 1.94 | 73.64 | 11.95 |
| 665 | 855 | 41.00 | 0.10 | 65.67 | 1.78 |
| 666 | 856 | 26.64 | 0.13 | 59.77 | 1.62 |
| 667 | 857 | 64.25 | 3.08 | 66.81 | 3.23 |
| 668 | 858 | 32.33 | 0.83 | 37.79 | 5.69 |
| 669 | 859 | 15.72 | 0.59 | 34.59 | 0.14 |
| 670 | 860 | 32.74 | ND | 27.63 | 0.65 |
| 671 | 861 | 30.19 | 1.51 | 33.68 | 1.20 |
| 672 | 862 | ND | ND | 41.11 | 1.83 |
| 673 | 863 | 65.65 | 2.85 | 77.40 | 6.23 |
| 674 | 864 | 28.04 | 1.52 | 29.92 | 1.37 |
| 675 | 865 | 26.86 | 1.80 | 30.83 | 2.36 |
| 676 | 866 | 12.05 | 0.39 | 25.67 | 2.82 |
| 677 | 867 | 29.56 | 0.63 | 37.35 | 1.96 |
| 678 | 868 | 22.21 | 1.43 | 86.71 | 6.55 |
| 679 | 869 | 13.12 | 0.23 | 64.03 | 5.26 |
| 680 | 870 | 43.38 | 0.90 | 65.20 | 15.47 |
| 681 | 871 | 19.99 | 0.54 | 30.38 | 0.37 |
| 682 | 872 | 18.27 | 0.54 | 32.14 | 2.85 |
| 683 | 873 | 30.77 | 0.91 | 40.48 | 1.33 |
| 684 | 874 | 90.59 | 4.90 | 76.68 | 2.00 |
| 685 | 875 | 67.79 | 1.50 | 71.54 | 4.47 |
| 686 | 876 | 31.81 | 0.72 | 37.36 | 1.38 |
| 687 | 877 | 29.59 | 0.74 | 38.54 | 0.55 |
| 688 | 878 | 25.19 | 0.83 | 43.29 | 0.84 |
| 689 | 879 | 21.00 | 0.10 | 37.36 | 0.79 |
| 690 | 880 | 26.79 | 0.03 | 42.53 | 1.83 |
| 691 | 881 | 25.50 | 0.40 | 61.71 | 0.47 |
| 692 | 882 | 42.45 | 1.45 | 27.04 | 0.61 |
| 693 | 883 | 20.03 | 1.47 | 38.58 | 1.18 |
| 694 | 884 | 16.37 | 0.50 | 23.17 | 1.46 |
| 695 | 885 | 36.88 | 0.39 | 28.29 | 1.99 |
| 696 | 886 | 48.82 | 2.20 | 37.86 | 1.28 |
| 697 | 887 | 20.86 | 1.11 | 21.96 | 0.10 |
| 698 | 888 | 46.31 | 1.52 | 37.69 | 0.53 |
| 699 | 889 | 39.13 | 0.39 | 60.49 | 4.52 |
| 700 | 890 | 50.00 | 0.31 | 45.42 | 1.52 |
| 701 | 891 | 32.25 | 1.10 | 20.87 | 0.23 |
| 702 | 892 | 64.37 | 2.43 | 70.25 | 1.17 |
| 703 | 893 | 36.96 | 1.64 | 85.69 | 2.41 |
| 704 | 894 | 35.78 | 2.33 | 62.13 | 1.14 |
| 705 | 895 | 38.26 | 1.61 | 48.78 | 0.64 |
| 706 | 896 | 115.36 | 3.52 | 92.00 | 2.47 |
| 707 | 897 | 68.48 | 0.76 | 50.64 | 1.12 |
| 708 | 898 | 52.11 | 6.16 | 49.25 | 2.91 |
| 709 | 899 | 22.52 | ND | 33.36 | 3.69 |
| 710 | 900 | 34.74 | 1.75 | 23.83 | 0.27 |
| 711 | 901 | 46.21 | 1.01 | 52.28 | 0.03 |
| 712 | 902 | 90.81 | 6.06 | 58.63 | 2.87 |
| 713 | 903 | 34.50 | 0.60 | 39.43 | 3.26 |
| 714 | 904 | 42.15 | 5.60 | 65.37 | 12.74 |
| 715 | 905 | 58.15 | 1.68 | 54.41 | 5.88 |
| 716 | 906 | 32.39 | 2.23 | 26.11 | 2.57 |
| 717 | 907 | 72.48 | 0.75 | 54.77 | 1.73 |
| 718 | 908 | 24.63 | 1.73 | 25.32 | 1.49 |
| 719 | 909 | 49.30 | 5.47 | 40.18 | 5.18 |
| 720 | 910 | ND | ND | 49.46 | 5.42 |
| 721 | 911 | 60.40 | 3.12 | 53.10 | 1.48 |
| 722 | 912 | 20.71 | 0.24 | 26.97 | 0.24 |
| 723 | 913 | 75.32 | 0.67 | 51.05 | 1.48 |
| 724 | 914 | 51.01 | 2.80 | 30.61 | 1.30 |
| 725 | 915 | 23.08 | 0.02 | 23.32 | 3.21 |
| 726 | 916 | 18.66 | 1.98 | 61.31 | 1.30 |
| 727 | 917 | 67.43 | 4.44 | 58.84 | ND |
| 728 | 918 | 44.35 | 6.52 | 42.83 | 1.80 |
| 729 | 919 | 47.10 | 1.12 | 46.68 | 3.84 |
| 730 | 920 | 45.41 | 2.35 | 20.60 | 1.08 |
| 731 | 921 | 26.88 | 1.24 | 14.75 | 0.48 |
| 732 | 922 | 92.37 | 2.67 | 54.18 | 1.21 |
| 733 | 923 | 69.65 | ND | 50.60 | 3.20 |
| 734 | 924 | 24.95 | 1.98 | 29.13 | 0.49 |
| 735 | 925 | 58.24 | 6.09 | 37.94 | 0.03 |
| 736 | 926 | 31.05 | 2.26 | 17.51 | 0.90 |
| 737 | 927 | 10.94 | 1.16 | 9.66 | 0.29 |
| 738 | 928 | 26.59 | 0.45 | 63.00 | 3.94 |
| 739 | 929 | 28.28 | 0.83 | 48.21 | 1.29 |
| 740 | 930 | 32.97 | 1.94 | 46.95 | 0.41 |
| 741 | 931 | 63.31 | 0.58 | 59.26 | 0.22 |
| 742 | 932 | 102.22 | 4.06 | 71.06 | 3.14 |
| 743 | 933 | 116.69 | 1.94 | 52.06 | 0.91 |
| 744 | 934 | 88.66 | 6.99 | 48.42 | 4.38 |
| 745 | 935 | 10.53 | 0.31 | 15.56 | 1.10 |
| 746 | 936 | 23.87 | 0.73 | 36.53 | 0.08 |
| 747 | 937 | 40.13 | 0.28 | 56.89 | 2.73 |
| 748 | 938 | 46.26 | 3.22 | 39.70 | 3.25 |
| 749 | 939 | 26.34 | 0.26 | 61.16 | 0.65 |
| 750 | 940 | 19.07 | 0.38 | 24.44 | 2.82 |
| 751 | 941 | 13.73 | 0.27 | 17.87 | 0.69 |
| 752 | 942 | 48.27 | 1.78 | 14.80 | 1.02 |
| 753 | 943 | 81.59 | 1.23 | 48.98 | 1.47 |
| 754 | 944 | 69.15 | 3.62 | 49.62 | 6.01 |
| 755 | 945 | 87.80 | 4.89 | 52.87 | ND |
| 756 | 946 | 49.00 | 1.51 | 54.71 | 3.42 |
| 757 | 947 | 48.70 | 1.09 | 52.56 | 9.51 |
| 758 | 948 | 126.61 | 0.70 | 105.59 | 8.37 |
| 759 | 949 | 56.89 | 8.41 | 65.78 | 6.80 |
| 760 | 950 | 30.84 | 2.72 | 65.57 | 7.23 |
| 761 | 951 | 72.28 | 2.66 | 105.38 | 7.61 |
| 762 | 952 | 15.50 | 0.09 | 42.54 | 0.48 |
| 763 | 953 | 53.48 | 7.57 | 58.44 | 0.38 |
| 764 | 954 | 74.07 | 3.04 | 51.32 | 2.45 |
| 765 | 955 | 32.08 | 2.76 | 36.08 | 2.39 |
| 766 | 956 | 25.44 | 2.71 | 31.36 | 0.82 |
| 767 | 957 | 75.61 | ND | 55.97 | 3.27 |
| 768 | 958 | 53.62 | 0.44 | 62.13 | 0.97 |
| 769 | 959 | 75.11 | 7.01 | 75.26 | 15.47 |
| 770 | 960 | 89.94 | 1.74 | 90.85 | 5.35 |
| 771 | 961 | 16.76 | 1.69 | 34.84 | 4.30 |
| 772 | 962 | 54.87 | 0.22 | 58.28 | 5.87 |
| 773 | 963 | 44.97 | 3.60 | 62.58 | 2.48 |
| 774 | 964 | 112.84 | 2.38 | 100.26 | 4.44 |
| 775 | 965 | 103.57 | 7.01 | 77.71 | 3.90 |
| 776 | 966 | 70.23 | 0.44 | 56.96 | 2.00 |
| 777 | 967 | 26.87 | 2.25 | 48.77 | 3.13 |
| 778 | 968 | 48.67 | 1.24 | 46.70 | 0.81 |
| 779 | 969 | 110.30 | 0.88 | 115.21 | 10.85 |
| 780 | 970 | 132.73 | 0.00 | 126.35 | 1.37 |
| 781 | 971 | 114.50 | 2.07 | 87.77 | 11.57 |
| 782 | 972 | 104.57 | 0.82 | 113.05 | 1.83 |
| 783 | 973 | 27.51 | 0.68 | 33.33 | 1.41 |
| 784 | 974 | 16.25 | 0.78 | 25.93 | 3.90 |
| 785 | 975 | 90.20 | 4.55 | 66.84 | 7.52 |
| 786 | 976 | 63.15 | 1.20 | 60.61 | 3.09 |
| 787 | 977 | 68.93 | 4.67 | 48.44 | 4.13 |
| 788 | 978 | 10.25 | 0.46 | 12.13 | 0.16 |
| 789 | 979 | 15.84 | 0.09 | 21.17 | 0.36 |
| 790 | 980 | 100.54 | 4.78 | 66.60 | 2.70 |
| 791 | 981 | 35.84 | 2.04 | 30.72 | 0.25 |
| 792 | 982 | 9.78 | 0.83 | 11.67 | ND |

### Example 2. IC50 Determination of an siRNA Molecule of the Disclosure

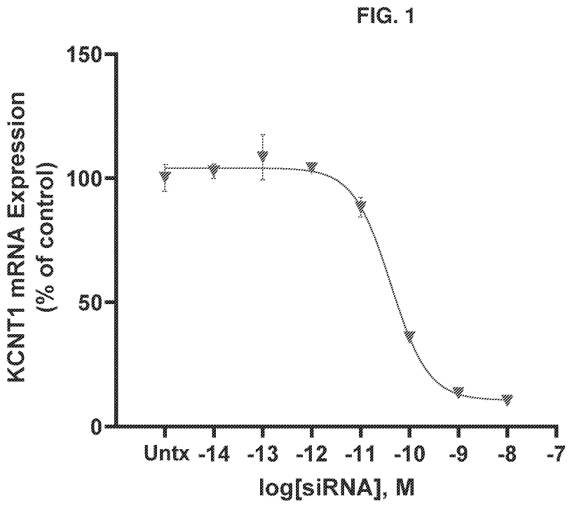

For compound potency analysis, HEK-KCNT1 cells were actively transfected with KCNT1 siRNA at concentrations of 10 fM to 100 nM and the IC50 was calculated. Cells were dosed with siRNA for 72 hours prior to lysis and downstream analysis. Expression was determined using the method described above. An siRNA molecule have an antisense strand of SEQ ID NO: 19 and a sense strand of SEQ ID NO: 237 were tested in this assy. The IC50 was determined to be 0.0415. The IC50 curve is shown in FIG. 1.

### Example 3. Generating KCNT1-Targeting siRNA Molecules

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the sodium-activated potassium channel subfamily T member 1 (KCNT1) mRNA target sequence, are shown in Table 1, above. It is appreciated that one of skill in the art could anneal the antisense (AS) strand to the corresponding sense(S) strand to yield a ds-siRNA molecule. Alternatively, one of skill in the art could derive a ss-siRNA molecule using antisense strand only.

### Example 4. Optimizing KCNT1-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl(2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

SiRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

SIRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

SiRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 5. Preparation and Administrating KCNT1-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject's CNS that is tolerated by the siRNA compositions of the disclosure. Non-limiting examples of siRNA injections into the CNS include intrathecally, intracerebroventricularly, or intra-cisterna magna injection by catheterization. A physician having ordinary skill in the art can readily determine an effective route of administration.

### Example 6. Methods for the Treatment of Epilepsy Using KCNT1-Targeting siRNA Molecules

A subject in need of treatment for epilepsy and/or epileptic-associated phenotypes (e.g., seizures, perioral cyanosis, flushing, apnea, hypertonia, pulmonary hemorrhage, or cardiac arrythmia) is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of KCNT1 mRNA or suitable biomarker) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, or by intra-cisterna magna injection via catheterization) and condition at a rate tolerable to the patient until the subject has reached a maximum tolerated dose, or until symptoms are ameliorated satisfactorily.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within sodium-activated potassium channel subfamily T member 1 (KCNT1) mRNA transcript having the nucleic acid sequence of any one of SEQ ID NOs: 437-654 and 1035-1224.

2. The siRNA molecule of claim 1, wherein the antisense strand has at least 70% complementarity to a region of 19, 20, 21, or more contiguous nucleobases within the KCNT1 mRNA transcript having the nucleic acid sequence of any one of SEQ ID NOs: 437-654 and 1035-1224, optionally wherein the antisense strand has at least 70% complementarity to the KCNT1 mRNA transcript having the nucleic acid sequence of any one of SEQ ID NOs: 437-654 and 1035-1224.

3. The siRNA molecule of claim 2, wherein the antisense strand has at least 75% complementarity to the region within the KCNT1 mRNA transcript having the nucleic acid sequence of any one of SEQ ID NOS: 437-654 and 1035-1224, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the KCNT1 mRNA transcript having the nucleic acid sequence of any one of SEQ ID Nos: 437-654 and 1035-1224.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

5. The siRNA molecule of claim 4, wherein the antisense strand comprises from 10 to 30 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

6. The siRNA molecule of claim 5, wherein the antisense strand comprises from 12 to 30 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

7. The siRNA molecule of claim 6, wherein the antisense strand comprises from 15 to 30 contiguous nucleotides that are fully complementary to a contiguous polynucleotide segment of equal length within the region of the KCNT1 RNA transcript having the nucleic acid sequence of any one of SEQ ID NOS: 437-654 and 1035-1224.

8. The siRNA molecule of claim 7, wherein the antisense strand comprises from 18 to 30 contiguous nucleotides that are fully complementary to a contiguous polynucleotide segment of equal length within the region of the KCNT1 RNA transcript having the nucleic acid sequence of any one of SEQ ID NOS: 437-654 and 1035-1224.

9. The siRNA molecule of claim 8, wherein the antisense strand comprises from 18 to 25 contiguous nucleotides that are fully complementary to a contiguous polynucleotide segment of equal length within the region of the KCNT1 RNA transcript having the nucleic acid sequence of any one of SEQ ID Nos: 437-654 and 1035-1224.

10. The siRNA molecule of any one of claims 1-9, wherein the antisense strand comprises from 18 to 21 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

11. The siRNA molecule of claim 9, wherein the antisense strand comprises 21 contiguous nucleotides that are fully complementary to a contiguous polynucleotide segment of equal length within the region of the KCNT1 RNA transcript having the nucleic acid sequence of any one of SEQ ID NOs: 437-654 and 1035-1224.

12. The siRNA molecule of any one of claims 1-11, wherein the antisense strand comprises 9 or fewer nucleotide mismatches relative to the region of the KCNT1 RNA transcript having the nucleic acid sequence of any one of SEQ ID NOs: 437-654 and 1035-1224, optionally wherein the antisense strand comprises 8 or fewer, 7 or fewer, 6 or fewer, 5 or fewer, 4 or fewer, 3 or fewer, 2 or fewer, or only 1 mismatch relative to the region of the KCNT1 RNA transcript having the nucleic acid sequence of any one of SEQ ID NOs: 437-654 and 1035-1224.

13. The siRNA molecule of any one of claims 1-12, wherein the antisense strand has a nucleic acid sequence that is at least 85% identical to the nucleic acid sequence of any one of SEQ ID NOs: 1-218 and 655-844.

14. The siRNA molecule of claim 13, wherein the antisense strand has a nucleic acid sequence that is at least 90% identical to the nucleic acid sequence of any one of SEQ ID NOs: 1-218 and 655-844.

15. The siRNA molecule of claim 14, wherein the antisense strand has a nucleic acid sequence that is at least 95% identical to the nucleic acid sequence of SEQ ID NOs: 1-218 and 655-844, optionally wherein the antisense strand has a nucleic acid sequence that is at least 96%, 97%, 98%, or 99% identical to the nucleic acid sequence of any one of SEQ ID NOs: 1-218 and 655-844.

16. The siRNA molecule of claim 15, wherein the antisense strand has the nucleic acid sequence of any one of SEQ ID NOs: 1-218 and 655-844.

17. The siRNA molecule of any one of claims 1-16, wherein the sense strand has a nucleic acid sequence that is at least 85% identical to the nucleic acid sequence of any one of SEQ ID NOs: 219-436 and 845-1034.

18. The siRNA molecule of claim 17, wherein the sense strand has a nucleic acid sequence that is at least 90% identical to the nucleic acid sequence of any one of SEQ ID NOs: 219-436 and 845-1034.

19. The siRNA molecule of claim 18, wherein the sense strand has a nucleic acid sequence that is at least 95% identical to the nucleic acid sequence of SEQ ID NOs: 219-436 and 845-1034, optionally wherein the sense strand has a nucleic acid sequence that is at least 96%, 97%, 98%, or 99% identical to the nucleic acid sequence of any one of SEQ ID NOs: 219-436 and 845-1034.

20. The siRNA molecule of claim 19, wherein the sense strand has the nucleic acid sequence of any one of SEQ ID NOs: 219-436 and 845-1034.

21. The siRNA molecule of any one of claims 1-20, wherein the antisense strand comprises a structure represented by Formula I, wherein Formula I is, in the 5′- to -3′ direction: 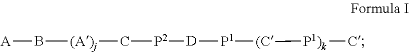
wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

22. The siRNA molecule of claim 21, wherein the antisense strand comprises a structure represented by Formula A1, wherein Formula A1 is, in the 5′- to -3′ direction: 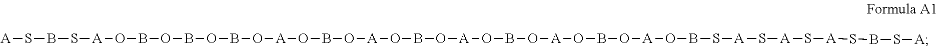
wherein A represents a 2′-O-Me ribonucleoside, B represents a 2′-F ribonucleoside, O represents a phosphodiester internucleoside linkage, and S represents a phosphorothioate internucleoside linkage.

23. The siRNA molecule of any one of claims 1-20, wherein the antisense strand comprises a structure represented by Formula II, wherein Formula II is, in the 5′- to -3′ direction: 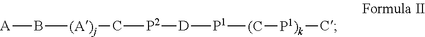
wherein A is represented by the formula C-P¹-D-P¹;
each A′ is represented by the formula C-P²-D-P²;
B is represented by the formula C-P²-D-P²-D-P²-D-P²;
each C is a 2′-O-methyl(2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

24. The siRNA molecule of claim 23, wherein the antisense strand comprises a structure represented by Formula A2, wherein Formula A2 is, in the 5′- to -3′ direction: 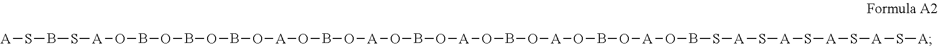
wherein A represents a 2′-O-Me ribonucleoside, B represents a 2′-F ribonucleoside, O represents a phosphodiester internucleoside linkage, and S represents a phosphorothioate internucleoside linkage.

25. The siRNA molecule of any one of claims 1-24, wherein the sense strand comprises a structure represented by Formula III, wherein Formula III is, in the 5′- to -3′ direction: 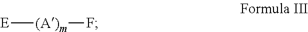
wherein E is represented by the formula (C-P¹)₂;
F is represented by the formula (C-P²) 3-D-P¹-C-P¹-C, (C-P²) 3-D-P²-C-P²-C, (C-P²) 3-D-P¹-C-P¹-D, or (C-P²) 3-D-P²-C-P²-D;
A′, C, D, P¹, and P² are as defined in Formula II; and
m is an integer from 1 to 7.

26. The siRNA molecule of claim 25, wherein the sense strand comprises a structure represented by Formula S1, wherein Formula S1 is, in the 5′- to -3′ direction: 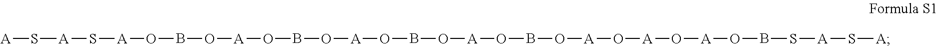
wherein A represents a 2′-O-Me ribonucleoside, B represents a 2′-F ribonucleoside, O represents a phosphodiester internucleoside linkage, and S represents a phosphorothioate internucleoside linkage.

27. The siRNA molecule of claim 25, wherein the sense strand comprises a structure represented by Formula S2, wherein Formula S2 is, in the 5′- to -3′ direction: 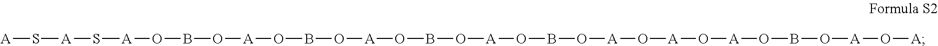
wherein A represents a 2′-O-Me ribonucleoside, B represents a 2′-F ribonucleoside, O represents a phosphodiester internucleoside linkage, and S represents a phosphorothioate internucleoside linkage.

28. The siRNA molecule of claim 25, wherein the sense strand comprises a structure represented by Formula S3, wherein Formula S3 is, in the 5′- to -3′ direction: 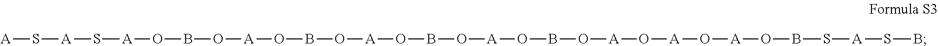
wherein A represents a 2′-O-Me ribonucleoside, B represents a 2′-F ribonucleoside, O represents a phosphodiester internucleoside linkage, and S represents a phosphorothioate internucleoside linkage.

29. The siRNA molecule of claim 25, wherein the sense strand comprises a structure represented by Formula S4, wherein Formula S4 is, in the 5′- to -3′ direction: 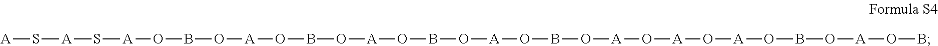
wherein A represents a 2′-O-Me ribonucleoside, B represents a 2′-F ribonucleoside, O represents a phosphodiester internucleoside linkage, and S represents a phosphorothioate internucleoside linkage.

30. The siRNA molecule of any one of claims 1-20 and 25-29, wherein the antisense strand comprises a structure represented by Formula IV, wherein Formula IV is, in the 5′- to -3′ direction: 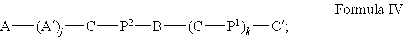
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

31. The siRNA molecule of claim 30, wherein the antisense strand comprises a structure represented by Formula A3, wherein Formula A3 is, in the 5′- to -3′ direction: 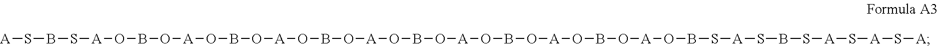
wherein A represents a 2′-O-Me ribonucleoside, B represents a 2′-F ribonucleoside, O represents a phosphodiester internucleoside linkage, and S represents a phosphorothioate internucleoside linkage.

32. The siRNA molecule of any one of claims 1-24, 30, and 31, wherein the sense strand comprises a structure represented by Formula V, wherein Formula V is, in the 5′- to -3′ direction: 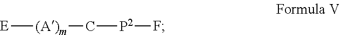
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

33. The siRNA molecule of claim 32, wherein the sense strand comprises a structure represented by Formula S5, wherein Formula S5 is, in the 5′- to -3′ direction: 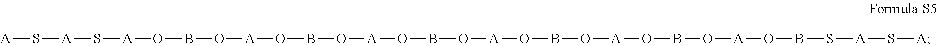
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of claim 32, wherein the sense strand comprises a structure represented by Formula S6, wherein Formula S6 is, in the 5′- to -3′ direction: 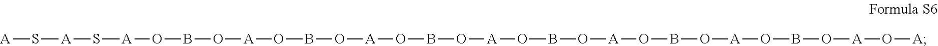
wherein A represents a 2′-O-Me ribonucleoside, B represents a 2′-F ribonucleoside, O represents a phosphodiester internucleoside linkage, and S represents a phosphorothioate internucleoside linkage.

35. The siRNA molecule of claim 32, wherein the sense strand comprises a structure represented by Formula S7, wherein Formula S7 is, in the 5′- to -3′ direction: 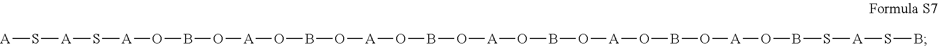
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of claim 32, wherein the sense strand comprises a structure represented by Formula S8, wherein Formula S8 is, in the 5′- to -3′ direction: 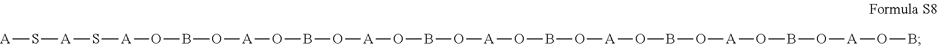
wherein A represents a 2′-O-Me ribonucleoside, B represents a 2′-F ribonucleoside, O represents a phosphodiester internucleoside linkage, and S represents a phosphorothioate internucleoside linkage.

37. The siRNA molecule of any one of claims 1-20, 25-29 and 32-36, wherein the antisense strand comprises a structure represented by Formula VI, wherein Formula VI is, in the 5′- to -3′ direction: 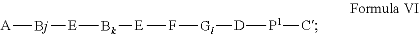
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7;
k is an integer from 1 to 7; and
I is an integer from 1 to 7.

38. The siRNA molecule of claim 37, wherein the antisense strand comprises a structure represented by Formula A4, wherein Formula A4 is, in the 5′- to -3′ direction: 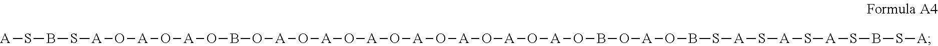
wherein A represents a 2′-O-Me ribonucleoside, B represents a 2′-F ribonucleoside, O represents a phosphodiester internucleoside linkage, and S represents a phosphorothioate internucleoside linkage.

39. The siRNA molecule of any one of claims 1-24, 30, 31, 37, and 38, wherein the sense strand comprises a structure represented by Formula VII, wherein Formula VII is, in the 5′- to -3′ direction: 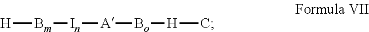
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

40. The siRNA molecule of claim 39, wherein the sense strand comprises a structure represented by Formula S9, wherein Formula S9 is, in the 5′- to -3′ direction: 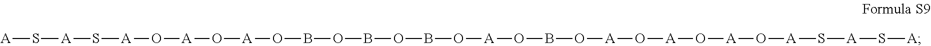
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of any one of claims 1-40, wherein the antisense strand further comprises a 5′ phosphorus stabilizing moiety at the 5′ end of the antisense strand.

42. The siRNA molecule of any one of claims 1-41, wherein the sense strand further comprises a 5′ phosphorus stabilizing moiety at the 5′ end of the sense strand.

43. The siRNA molecule of claim 41 or 42, wherein each 5′ phosphorus stabilizing moiety is, independently, represented by any one of Formulas IX-XVI: 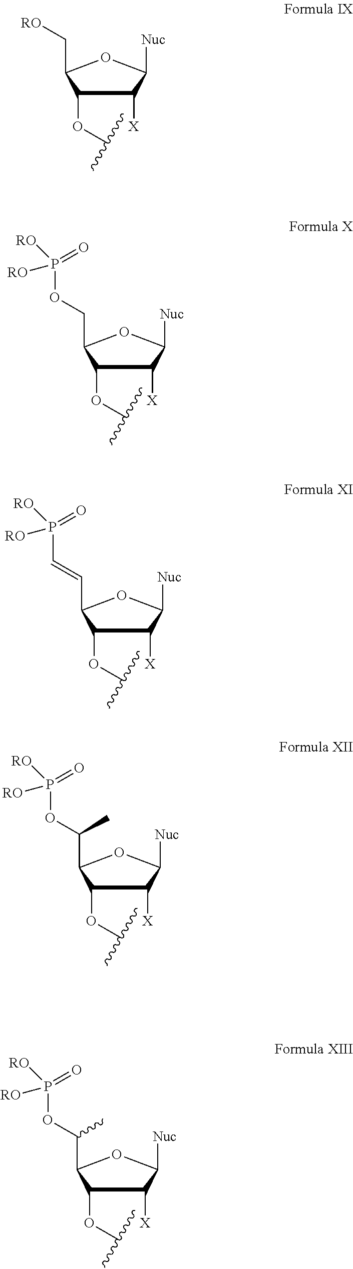 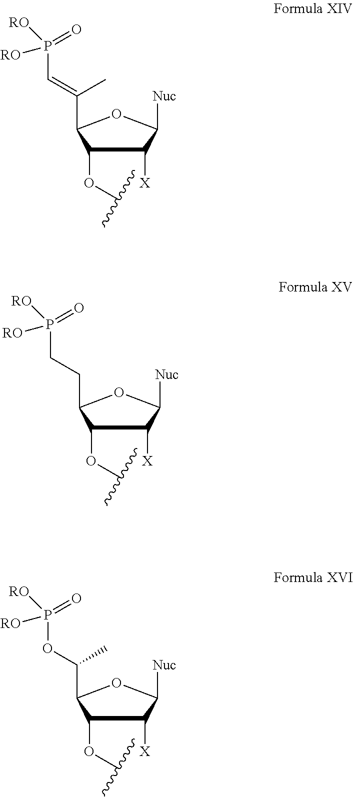
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

44. The siRNA molecule of claim 43, wherein the nucleobase is an adenine, uracil, guanine, thymine, or cytosine.

45. The siRNA molecule of any one of claims 41-44, wherein the 5′ phosphorus stabilizing moiety is (E)-vinylphosphonate represented by Formula XI.

46. The siRNA molecule of any one of claims 1-45, wherein the siRNA molecule further comprises a hydrophobic moiety at the 5′ or the 3′ end of the siRNA molecule.

47. The siRNA molecule of claim 46, wherein the hydrophobic moiety is selected from a group consisting of cholesterol, vitamin D, or tocopherol.

48. The siRNA molecule of any one of claims 1-47, wherein the length of the sense strand is between 10 and 30 nucleotides.

49. The siRNA molecule of claim 48, wherein the length of the sense strand is between 10 and 25 nucleotides.

50. The siRNA molecule of claim 49, wherein the length of the sense strand is between 12 and 25 nucleotides.

51. The siRNA molecule of claim 50, wherein the length of the sense strand is between 12 and 20 nucleotides.

52. The siRNA molecule of claim 51, wherein the length of the sense strand is between 12 and 19 nucleotides.

53. The siRNA molecule of claim 52, wherein the length of the sense strand is 15 nucleotides.

54. The siRNA molecule of claim 52, wherein the length of the sense strand is 16 nucleotides.

55. The siRNA molecule of claim 52, wherein the length of the sense strand is 18 nucleotides.

56. The siRNA molecule of any one of claims 1-55, wherein the length of the antisense strand is between 10 and 30 nucleotides.

57. The siRNA molecule of claim 56, wherein the length of the antisense strand is between 12 and 30 nucleotides.

58. The siRNA molecule of claim 57, wherein the length of the antisense strand is between 15 and 30 nucleotides.

59. The siRNA molecule of claim 58, wherein the length of the antisense strand is between 18 and 30 nucleotides.

60. The siRNA molecule of claim 59, wherein the length of the antisense strand is between 18 and 25 nucleotides.

61. The siRNA molecule of claim 60, wherein the length of the antisense strand is between 18 and 21 nucleotides.

62. The siRNA molecule of claim 61, wherein the length of the antisense strand is 18 nucleotides.

63. The siRNA molecule of claim 61, wherein the length of the antisense strand is 20 nucleotides.

64. The siRNA molecule of claim 61, wherein the length of the antisense strand is 21 nucleotides.

65. The siRNA molecule of any one of claims 1-64, wherein the siRNA molecule is a branched siRNA molecule.

66. The siRNA molecule of claim 65, wherein the branched siRNA molecule is di-branched, tri-branched, or tetra-branched.

67. The siRNA molecule of claim 66, wherein the siRNA molecule is a di-branched siRNA molecule, optionally wherein the di-branched siRNA molecule is represented by any one of Formulas XVII-XIX: 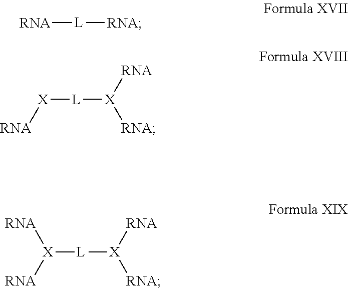
wherein each RNA is, independently, an siRNA molecule, L is a linker, and each X, independently, represents a branch point moiety.

68. The siRNA molecule of claim 66, wherein the siRNA molecule is a tri-branched siRNA molecule, optionally wherein the tri-branched siRNA molecule is represented by any one of Formulas XX-XXIII: 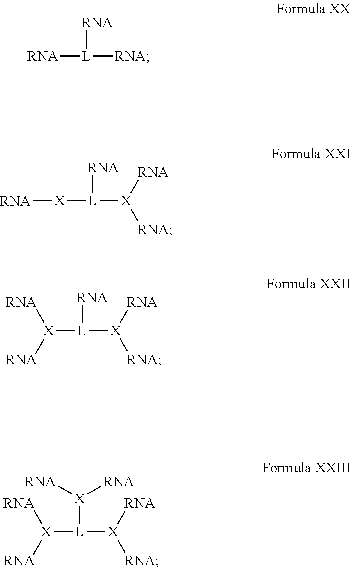
wherein each RNA is, independently, an siRNA molecule, L is a linker, and each X, independently, represents a branch point moiety.

69. The siRNA molecule of claim 66, wherein the siRNA molecule is a tetra-branched siRNA molecule, optionally wherein the tetra-branched siRNA molecule is represented by any one of Formulas XXIV-XXVIII: 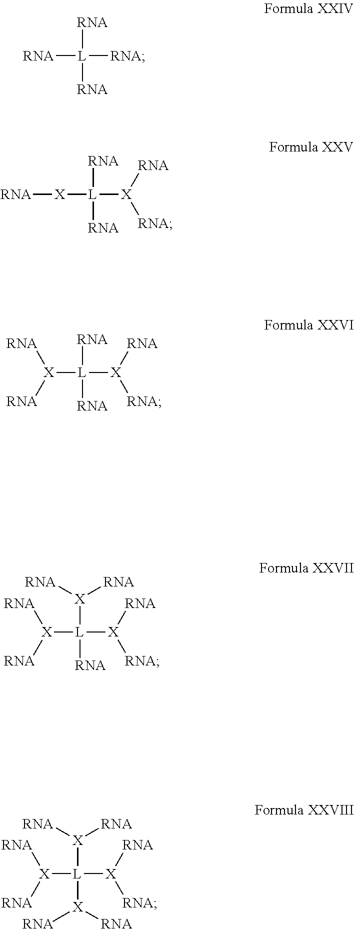
wherein each RNA is, independently, an siRNA molecule, L is a linker, and each X, independently, represents a branch point moiety.

70. The siRNA molecule of any one of claims 67-69, wherein the linker is selected from a group consisting of one or more contiguous subunits of an ethylene glycol, alkyl, carbohydrate, block copolymer, peptide, RNA, and DNA.

71. The siRNA molecule of claim 70, wherein the one or more contiguous subunits is 2 to 20 contiguous subunits.

72. A pharmaceutical composition comprising the siRNA molecule of any one of claims 1-71 and a pharmaceutically acceptable excipient, carrier, or diluent.

73. A method of delivering an siRNA molecule to the central nervous system (CNS) of a subject diagnosed as having an epilepsy syndrome, the method comprising administering a therapeutically effective amount of the siRNA molecule of any one of claims 1-71 or the pharmaceutical composition of claim 72 to the subject.

74. A method of treating an epilepsy syndrome in a subject in need thereof, the method comprising administering a therapeutically effective amount of the siRNA molecule of any one of claims 1-71 or the pharmaceutical composition of claim 72 to the subject.

75. The method of claim 73 or 74, wherein the epilepsy syndrome is frontal lobe epilepsy, occipital lobe epilepsy, medial temporal lobe epilepsy, parietal lobe epilepsy, benign myoclonic epilepsy in infants, juvenile myoclonic epilepsy, childhood absence epilepsy, juvenile absence epilepsy, epilepsy with generalized tonic clonic seizures in childhood, infantile spasms, Lennox-Gastaut syndrome, West syndrome, sleep-related hypermotor epilepsy, progressive myoclonus epilepsies, febrile fits, epilepsy with continuous spike and waves in slow wave sleep, Laudau Kleffner syndrome, Rasmussen's syndrome, epilepsy arising from an inborn error in metabolism, epilepsy of infancy with migrating focal seizures, autosomal dominant nocturnal frontal lobe epilepsy, Ohtahara syndrome, early myoclonic encephalopathy, focal epilepsy, and/or multifocal epilepsy.

76. A method of reducing potassium KCNT1 expression in a subject in need thereof, the method comprising administering a therapeutically effective amount of the siRNA molecule of any one of claims 1-71 or the pharmaceutical composition of claim 72 to the CNS of the subject.

77. The method of claim 76, wherein, upon administration of the siRNA molecule or pharmaceutical composition to the subject, the subject exhibits selective reduction in KCNT1 expression over expression of one or more other voltage-gated potassium ion channel genes.

78. The method of any one of claims 73-77, wherein the siRNA molecule or the pharmaceutical composition is administered to the subject by way of intracerebroventricular or intrathecal injection.

79. The method of any one of claims 73-78, wherein the subject is a human.

80. A kit comprising the siRNA molecule of any one of claims 1-71, or the pharmaceutical composition of claim 72, and a package insert, wherein the package insert instructs a user of the kit to perform the method of any one of claims 73-79.
